(19) United States
(12) Patent Application Publication
(10) Pub. No.: US 2010/0298611 A1
(43) Pub. Date: Nov. 25, 2010

(54) PRODUCTION OF FERMENTIVE END PRODUCTSFROM CLOSTRIDIUM sp.

(75) Inventors: Sarad Parekh, Grafton, MA (US); Khursheed Karim, Amherst, MA (US); John Kilbane, Houston, TX (US)

Correspondence Address: WILSON, SONSINI, GOODRICH & ROSATI, 650 PAGE MILL ROAD, PALO ALTO, CA, 94304-1050, US

(73) Assignee: Qteros, Inc., Marlborough, MA (US)

(21) Appl. No.: 12/720,574

(22) Filed: Mar. 9, 2010

Related U.S. Application Data
(60) Provisional application No. 61/158,581, filed on Mar. 9, 2009.
(60) Provisional application No. 61/158,600, filed on Mar. 9, 2009.
(60) Provisional application No. 61/171,077, filed on Apr. 20, 2009.

(51) Int. Cl. C12P 7/06; C12N 1/20; C12M 1/00; C07C 31/08

(52) U.S. Cl. 568/840; 435/161; 435/252.7; 435/289.1

(57) ABSTRACT

In one aspect, methods to enhance the production of ethanol and other fermentive end products from a wide variety of feedstocks by Clostridium microorganisms, such as Clostridium phytofermentans are disclosed. A method of improving fermentation performance of Clostridium microorganisms, such as Clostridium phytofermentans through the use of a fed-batch strategy is described, as well as methods of producing fermentive end products, such as alcohols and/or chemicals by fermenting Clostridium microorganisms, such as Clostridium phytofermentans in the presence of fatty acid-containing compounds and/or at reduced pH.

### CROSS-REFERENCE TO RELATED APPLICATIONS

This application claims priority to U.S. Provisional Application Ser. No. 61/158,581, filed Mar. 9, 2009, U.S. Provisional Application Ser. No. 61/158,600, filed Mar. 9, 2009, U.S. Provisional Application Ser. No. 61/171,077, filed Apr. 20, 2009 each of which is herein incorporated by reference in their entirety.

### BACKGROUND

Increasing cost of petroleum-based transportation fuels, dwindling petroleum reserves and concerns over the environmental impact of petroleum-fuel combustion are driving a strong demand for viable alternatives to replace petroleum-based fuels. In particular, recent years have highlighted the promise of producing biofuels through bio-conversion of a variety of pretreated biomass material, such as lignocellulosic material, starch, or agriculture waste/byproducts, in combination with enzymes and yeast/bacterial systems. A particular challenge is developing technology with the potential to economically convert polysaccharide containing materials such as woody or nonwoody plant material, as well as waste materials and side products from the processing of plant matter into high value transportation fuels and other energy forms or chemical feedstocks. Various examples of these polysaccharide containing materials include cellulosic, lignocellulosic, and hemicellulosic material; pectin containing material; starch; wood; corn stover; switchgrass; paper; and paper pulp sludge.

Some processes for converting these polysaccharide containing materials into biofuels such as ethanol require first the conversion of pretreated biomass substrates such as starch or cellulose containing materials into simple sugars (saccharification) through, for example, enzymatic hydrolysis, and the subsequent conversion (fermentation) of these simple sugars into biofuels such as ethanol through fermentation by yeasts. However, current bioconversion technologies have faced problems of high production costs and diversion of agricultural products from the food supply.

In some fermentations for production of ethanol, a simple sugar, such as sucrose, is obtained and fermented directly into ethanol. Such processes are used, for example, in Brazil to convert cane sugar to fuel grade ethanol. These processes are limited geographically to where simple sugar sources are inexpensive, such as in sugarcane growing regions. Additionally, these processes carry the undesirable aspect of diverting a valuable food source, such as sugar, to industrial rather than food uses.

Some fermentations for the production of ethanol utilize material that first requires hydrolysis, or conversion into less complex or lower molecular weight sugars prior to the conversion to ethanol. Such processes are frequently described for the production of corn ethanol, with the starch derived from corn being broken down, for example by added enzymes, and then finally converted to ethanol with organisms such as a Saccharomyces or Zymomonas species. Use of other materials, such as cellulosic, hemicellulosic or lignocellulosic materials also frequently require hydrolysis with added enzymes or by other chemicals/thermal means is the subject of much research, but little historical success.

The use of these enzymes which are added to the process is undesirable from both a cost standpoint and due to the fact that the processor is generally limited to those enzymes which are readily available commercially. Historically, the enzymes available commercially have been selected for processes such as conversion of starch to simple sugars such as glucose or fructose, laundry applications, and cereal foods. They are generally highly specialized, meaning that a single enzyme generally cannot be used with the widely varying feed material. Instead a number of enzymes are frequently used and combined into an “enzyme cocktail.” Broader activity is achieved with such mixtures, however this broader activity can come with a significantly higher price tag, as only a portion of the enzymes being added may be useful with the particular substrate being used in any one particular batch. Other enzymes, which are a part of the cocktail, may not be active on one substrate but are included in the mixture to provide usefulness for other feed substrates that may be used. As a result, in any one particular batch at least a portion of the enzymes added may not significantly contribute to the processing and are wasted.

Therefore, a fermentation process for producing ethanol or other desirable products from various feedstocks with high yield and productivity is desirable.

Ethanol fermentation from biomass including cellulosic, lignocellulosic, pectin, polyglucose and/or polyfructose containing biomass can provide much needed solutions for the world energy problem. Species of yeast, fungi and bacteria have been reported to be able to convert cellulosic biomass of its monomeric sugars to ethanol. However, many of these microorganisms produce ethanol only to low concentrations. This limitation can be due to a general lack of tolerance to ethanol by the organism, or a feedback inhibition or suppression mechanism present in the organism, or to some other mechanism as well as some combination of these mechanisms. Such ethanol production limitations can in addition to affecting the ethanol titer, can also affect the ethanol productivity.

A number of wild type and genetically improved microorganisms have been described for alcohol production by fermentation. Among these are Thermoanaerobacter ethanolicus, Clostridium thermocellum, Clostridium beijerinickii, Clostridium acetobutylicum, Clostridium tyrobutyricum, Clostridium thermobutyricum, Thermoanaerobacterium saccharolyticum, Thermoanaerobacter thermohydrosulfuricus, and Saccharomyces cerevisiae, Clostridium acetobutylicum, Moorella ssp., Carboxydocella ssp., Zymomonas mobilis, recombinant E. Coli, Klebsiella oxytoca and Clostridium beijerickii as well as other microorganisms. Difficulties in using these or other microorganisms for industrial scale alcohol production can include cell toxicity at relatively low alcohol concentrations, reduced cell growth or viability at relatively low alcohol concentrations, low alcohol titer, or low alcohol productivity. Alcohol tolerance is highly species and strain dependent. For example, in some fermentation processes, alcohol production can slow down or stop completely at around 10-20 g/L of alcohol. Some organisms die or are severely impaired at around 20 g/L of alcohol, such as ethanol.

### SUMMARY OF THE INVENTION

In one aspect, provided herein is a method for producing a fermentive end-product comprising: culturing a medium comprising Clostridium for a first period of time under conditions suitable for production of a fermentive end-product by said; adding one or more nutrients to the medium comprising Clostridium while prior to harvesting the fermentive end product; culturing a medium comprising Clostridium for a second period of time; and harvesting a fermentive end-product from the medium. In one embodiment, the Clostridium strain is Clostridium phytofermentans. In another embodiment, the fermentive end-product is ethanol. In another embodiment, the medium comprises a cellulosic and/or lignocellulosic material. In another embodiment, the cellulosic or lignocellulosic material is not enzymatically treated with a sufficient quantity of enzymes to convert more than 15% of the cellulosic or lignocellulosic material to simple sugars within 24 hours.

In one aspect, provided herein is a method of producing a fermentive end product comprising the steps of: culturing a strain of Clostridium phytofermentans in a medium; maintaining the total concentration of sugar compounds in the medium at least about 18 g/L; and harvesting a fermentive end-product from the medium. In one embodiment, maintaining the total concentration of sugar compounds comprises adding one or more medium components, at least one of which comprises one or more sugar compounds to the medium at least once during the culturing, wherein the medium components are added to a vessel containing the culture. In another embodiment, the total concentration of sugar compounds in the medium is maintained within the range from about 1 g/L to about 100 g/L for a portion of the culturing. In another embodiment, the total concentration of sugar compounds in the medium varies by less than about 25% during the period of fermentive end product production. In another embodiment, the fermentive end-product is ethanol. In another embodiment, further comprising adding a medium component comprising one or more nitrogen-containing material to the medium at least once during the fermentation, and wherein the medium component is added to a vessel containing the culture. In another embodiment, one or more of the medium components comprises one or more nitrogen-containing material. In another embodiment, the medium comprises a cellulosic or lignocellulosic material. In another embodiment, the cellulosic or lignocellulosic material is not enzymatically treated with a sufficient quantity of enzymes to convert more than 15% of the cellulosic or lignocellulosic material to simple sugars within 24 hours.

In one aspect, provided herein is a method of producing a fermentive end product, the method comprising the steps of: culturing a strain of Clostridium in a medium; and adding one or more medium components to the medium during the culturing of the Clostridium wherein one or more of the medium components comprises one or more sugar compounds, and the one or more sugar compounds are added in relation to an amount of sugar converted by the Clostridium to other compounds. In one embodiment, one or more of the medium components comprises a nitrogen source. In another embodiment, the nitrogen source includes proline, glycine, histidine, and/or isoleucine. In another embodiment, the medium components comprise a cellulosic or lignocellulosic material. In another embodiment, the cellulosic or lignocellulosic material is not enzymatically treated with a sufficient quantity of enzymes to convert more than 15% of the cellulosic or lignocellulosic material to simple sugars within 24 hours.

In one aspect, provided herein is a method of producing a fermentive end product, the method comprising: adding a first inoculum of a strain of Clostridium to a medium; culturing the Clostridium under conditions suitable for production of ethanol; adding additional viable cells of Clostridium sp. to the medium more than five hours after the first inoculum of Clostridium is added to the medium; and harvesting the fermentive end product from the medium. In one embodiment, the method further comprises adding one or more media components to the medium after adding the first inoculum of Clostridium. In another embodiment, an addition of media components and an addition of viable cells occurs sequentially or simultaneously.

In one aspect, provided herein is a method of producing ethanol, the method comprising the steps of: removing an impurity from an impure ethanol material to produce a purified ethanol material, wherein the purified ethanol material is more than about 90% (wt.) ethanol, and the impure ethanol material is derived from a fermentation medium made by culturing Clostridium phytofermentans cells in a fed batch culture, and wherein the ethanol concentration in the fermentation medium is greater than about 7 g/L.

In one aspect, provided herein is a method of producing a fermentive end product, the method comprising the steps of: culturing a medium comprising a strain of Clostridium phytofermentans, wherein the fermentive end product is produced at an instantaneous productivity of at least about 3 g/L-day.

In one aspect, provided herein is a method of producing a fermentive end product, comprising: providing a cellulosic material, wherein said cellulosic material has not been treated with exogenously supplied chemicals or enzymes; combining the cellulosic material with a microbe in a medium, wherein the medium does not comprise exogenously supplied enzymes; and fermenting the cellulosic material under conditions and for a time sufficient to produce a fermentive end product.

In one aspect, provided herein is a method of producing a fermentive end product, the method comprising: fermenting cells of Clostridium phytofermentans in the presence of a pH modifier, wherein a fermentive end product is produced. In one embodiment, the fermentive end product is ethanol. In another embodiment, fermenting the cells occurs at a pH, between about 6.0 to about 7.2. In another embodiment, the pH is about 6.5.

In one aspect, provided herein is a method of producing a fermentive end product, the method comprising: fermenting cells of a Clostridium strain in the presence of an added fatty acid material, wherein a fermentive end product is produced. In one embodiment, the fatty acid comprising material comprises one or more of corn oil, sunflower oil, safflower oil, canola oil, soybean oil, or rape seed oil. In another embodiment, the fatty acid comprising material comprises a phospholipid or a lysophospholipid.

In one aspect, provided herein is a fermentation medium, the medium comprising cells of Clostridium phytofermentans and a pH modifier, wherein a fermentive end product is produced.

In one aspect, provided herein is a fermentation medium, the medium comprising cells of a Clostridium strain and an added fatty acid containing compound, wherein a fermentive end product is produced.

In one aspect, provided herein is a fermentation medium comprising a strain of Clostridium phytofermentans, a nitrogen source comprising proline, glycine, histidine, and/or isoleucine, and a cellulosic or lignocellulosic material.

In one aspect, provided herein is a method of producing alcohol, the method comprising: fermenting cells of a Clostridium strain and the presence of a pH modifier and a fatty acid material, wherein a fermentive end product is produced.

In one aspect, provided herein is a fuel plant comprising a fermenter configured to house a medium and a strain of Clostridium phytofermentans, wherein said fermenter is configured to maintain an amount of sugar compounds at a level that varies by less than about 25% during fermentation.

In one aspect, provided herein is a fuel plant comprising a fermenter configured to house a medium and a strain of Clostridium phytofermentans, wherein said fermenter is configured to periodically supplement said medium with additional medium components or additional viable cells of Clostridium phytofermentans.

In one aspect, provided herein is a fuel plant comprising a fermenter configured to house a medium and a strain of Clostridium phytofermentans, wherein said medium comprises a pH modifier and a cellulosic or lignocellulosic material. In one embodiment, said medium further comprises a fatty acid material.

In one aspect, provided herein is a fuel plant comprising a fermenter configured to house a medium and a strain of Clostridium phytofermentans, wherein said medium comprises a nitrogen source comprising proline, glycine, histidine, and/or isoleucine, and a cellulosic or lignocellulosic material.

In one aspect, provided herein is a fuel plant comprising a fermenter configured to house a medium and a strain of Clostridium phytofermentans, wherein said medium comprises a fatty acid material and a cellulosic or lignocellulosic material.

In one aspect, provided herein is a fermentive end product produced by fermenting a cellulosic or lignocellulosic material with a strain of Clostridium phytofermentans, in a medium comprising an amount of sugar compounds at a level that varies by less than about 25% during fermentation.

In one aspect, provided herein is a fermentive end product produced by fermenting a cellulosic or lignocellulosic material with a strain of Clostridium phytofermentans, in a medium comprising a pH modifier.

In one aspect, provided herein is a fermentive end product produced by fermenting a cellulosic or lignocellulosic material with a strain of Clostridium phytofermentans, in a medium comprising a fatty acid.

In one aspect, provided herein is a fermentive end product produced by fermenting a cellulosic or lignocellulosic material with a strain of Clostridium phytofermentans, in a medium comprising a nitrogen source comprising proline, glycine, histidine, and/or isoleucine.

In another aspect of the invention, a method is disclosed for the production of ethanol. The method comprises (1) inoculating a growth medium with a strain of Clostridium phytofermentans to form a broth; (2) culturing the broth under conditions suitable for growth of the Clostridium phytofermentans and production of ethanol by Clostridium phytofermentans; (3) adding one or more nutrients to the broth while the Clostridium phytofermentans is present; and (4) continuing to culture the broth under conditions suitable for growth of the Clostridium phytofermentans and production of ethanol by Clostridium phytofermentans, wherein the ethanol is present in the broth at a concentration of about 5 g/L or more.

In one embodiment of the above-described process, the ethanol is present in the broth at a concentration of about 7 g/L or more. In another embodiment, the ethanol is present in the broth at a concentration of about 9 g/L or more. In another embodiment, the ethanol is present in the broth at a concentration of about 11 g/L or more. In another embodiment, the ethanol is present in the broth at a concentration of about 13 g/L or more. In another embodiment, the ethanol is present in the broth at a concentration of about 10-14 g/L.

In another embodiment, the growth medium comprises a cellulosic and/or lignocellulosic material. In another embodiment, the growth medium comprises a cellulosic or lignocellulosic material, wherein the cellulosic or lignocellulosic material was not enzymatically treated with a sufficient quantity of enzymes to convert more than 15% of the cellulosic or lignocellulosic material to simple sugars within 24 hours.

In another aspect, a process is disclosed in accordance with a preferred embodiment of the present invention for making ethanol. The process comprises (1) culturing a strain of Clostridium phytofermentans in a broth; (2) maintaining the total concentration of sugar compounds in the broth at more than about 18 g/L; and (3) producing ethanol at a concentration of about 10 g/L or more. In one embodiment of the above-described process, the broth at some time during the culturing comprises ethanol at more than about 7 g/L.

In another embodiment, maintaining the total concentration of sugar compounds comprises adding one or more medium supplements, at least one of which comprises one or more sugar compounds to the broth at least once during the culturing, wherein the medium supplements are added to a vessel containing the culture.

In another embodiment, the total concentration of sugar compounds in the broth is maintained at more than about 25 g/L for a portion of the culturing. In another embodiment, the total concentration of sugar compounds in the broth is maintained within the range from about 30 g/L to about 100 g/L for a portion of the culturing.

In another embodiment, maintaining the total concentration of sugar compounds comprises adding one or more medium supplements, at least one of which comprises one or more sugar compounds to the broth at least once during the culturing, and one or more of the medium supplements comprise phytate, wherein the medium supplements are added to a vessel containing the culture.

In another embodiment, the total concentration of sugar compounds in the broth is maintained for a period, wherein the period being at least about 10 hours.

In another embodiment, the total concentration of sugar compounds in the broth is maintained for a period, wherein the period being at least about 10 hours and the total concentration of sugar compounds in the broth varies by less than about 25% during the period.

In another embodiment, the process further comprises adding a medium supplement comprising one or more nitrogen-containing material to the broth at least once during the fermentation, and wherein the medium supplement is added to a vessel containing the culture.

In another embodiment, maintaining the total concentration of sugar compounds comprises adding one or more medium supplements, at least one of which comprises one or more sugar compounds to the broth at least once during the culturing, and one or more of the medium supplements comprises one or more nitrogen-containing materials, wherein the medium supplements are added to a vessel containing the culture.

In another embodiment, the broth comprises a cellulosic or lignocellulosic material. In another embodiment, the broth comprises a cellulosic or lignocellulosic material, and the cellulosic or lignocellulosic material was not enzymatically treated with a sufficient quantity of enzymes to convert more than 15% of the cellulosic or lignocellulosic material to simple sugars within 24 hours.

In another aspect, a process is disclosed in accordance with a preferred embodiment of the present invention for making ethanol. The process comprises (1) culturing a strain of Clostridium phytofermentans in a broth; and (2) adding one or more medium components to the broth during the culturing of the Clostridium phytofermentans wherein one or more of the medium supplements comprises one or more sugar compounds, and the one or more sugar compounds are added in relation to an amount of sugar converted by the Clostridium phytofermentans to other compounds, and ethanol is produced at greater than about 10 g/L.

In one embodiment of the above-described process, one or more of the medium components comprises a nitrogen source. In another embodiment, one or more of the medium components comprises a nitrogen source and the nitrogen source includes proline, glycine, histidine, and/or isoleucine. In another embodiment, one or more of the medium components comprises a nitrogen source, wherein the nitrogen source includes proline, glycine, histidine, and/or isoleucine, and the proline, glycine, histidine, or isoleucine is provided in an amount of at least 0.9 g/L.

In another embodiment, the culturing of Clostridium phytofermentans includes a growth phase, and at least a portion of the medium component is added to the broth during the growth phase.

In another embodiment, the culturing of Clostridium phytofermentans includes a stationary phase, and at least a portion of the medium supplement is added to the broth during the stationary phase.

In another aspect, a process is disclosed in accordance with a preferred embodiment of the present invention for making ethanol. The process comprises (1) culturing a broth comprising Clostridium phytofermentans under conditions suitable for production of ethanol; and (2) collecting ethanol produced by the Clostridium phytofermentans in the broth, wherein the concentration of ethanol in the broth is more than about 8 g/L. In one embodiment of the above-described process, the concentration of ethanol in the broth at some point during the culturing of the Clostridium phytofermentans is in the range of from about 8 to about 14 g/L.

In another aspect, a process is disclosed in accordance with a preferred embodiment of the present invention for making ethanol. The process comprises culturing a broth comprising Clostridium phytofermentans under conditions suitable for production of ethanol, wherein the broth comprises ethanol in a concentration of more than about 8 g/L.

In another aspect, a process is disclosed in accordance with a preferred embodiment of the present invention for making ethanol. The process comprises (1) adding a first inoculum of Clostridium phytofermentans to a medium to form a broth; (2) culturing the broth comprising Clostridium phytofermentans under conditions suitable for production of ethanol; (3) adding additional viable cells of Clostridium phytofermentans to the broth more than five hours after the first inoculum of Clostridium phytofermentans was added to the medium; and (4) continuing to culture the broth, wherein ethanol is produced at greater than about 8 g/L.

In one embodiment of the above-described process, the process further comprises adding one or more media components to the broth after adding the first inoculum of Clostridium phytofermentans.

In another embodiment, the process further comprises adding one or more media components to the broth after adding the first inoculum of Clostridium phytofermentans, and an addition of media components and an addition of viable cells occur sequentially or simultaneously.

In another aspect, a process is disclosed in accordance with a preferred embodiment of the present invention for making ethanol. The process comprises (1) removing an impurity from an impure ethanol material to produce a purified ethanol material, wherein the purified ethanol material is more than about 90% (wt.) ethanol, and the impure ethanol material is derived from a fermentation broth made by culturing Clostridium phytofermentans cells in a fed batch culture, and wherein the ethanol concentration in the fermentation broth was greater than about 7 g/L.

In one embodiment of the above-described process, the impurity removed from the impure ethanol material comprises water.

In another aspect, a process is disclosed in accordance with a preferred embodiment of the present invention for making ethanol. The process comprises (1) inoculating a medium with microorganisms of Clostridium phytofermentans to form a broth; (2) culturing the broth under conditions suitable for growth of the microorganisms and production of ethanol by the microorganisms; (3) increasing the broth volume by adding medium to the broth while the microorganisms are present; and (4) continuing to culture the broth under conditions suitable for growth of the microorganism and production of ethanol by the microorganisms, wherein the growth phase for the microorganisms is extended to more than about six hours.

In another aspect, a process is disclosed in accordance with a preferred embodiment of the present invention for making ethanol. The process comprises (1) culturing a broth comprising a strain of Clostridium phytofermentans, and a nitrogen source comprising proline, glycine, histidine, and/or isoleucine, under conditions suitable for production of ethanol at a concentration greater than or equal to about 8 g/L.

In one embodiment of the above-described process, proline, glycine, histidine, or isoleucine is provided in an amount of at least about 0.09 g/L. In another embodiment, at least a portion of the nitrogen source is obtained from corn steep liquor or corn steep powder. In another embodiment, the broth further comprises at least about 0.4 g/L phytate. In another embodiment, the broth further comprises a cellulosic or lignocellulosic material. In another embodiment, the broth further comprises a cellulosic or lignocellulosic material, wherein the cellulosic or lignocellulosic material was not enzymatically treated with a sufficient quantity of enzymes to convert more than 15% of the cellulosic or lignocellulosic material to simple sugars within 24 hours. In another embodiment, the broth further comprises at least about 0.4 g/L phytate, and the proline, glycine, histidine, or isoleucine is provided at a concentration of at least about 0.09 g/L.

In another aspect, a process is disclosed in accordance with a preferred embodiment of the present invention for making ethanol. The process comprises (1) culturing a broth comprising a strain of Clostridium phytofermentans, a nitrogen source, and phytate, wherein the phytate is present at a concentration of about 0.4 g/L or higher, under conditions suitable for production of ethanol at a concentration greater than or equal to about 8 g/L.

In another aspect, a process is disclosed in accordance with a preferred embodiment of the present invention for making ethanol. The process comprises (1) culturing a broth comprising a strain of Clostridium phytofermentans, wherein the ethanol is produced at an instantaneous productivity of at least about 3 g/L-day. In one embodiment of the process, the ethanol is produced at an instantaneous rate of about 3 g/L-day to about 15 g/L-day. In another embodiment, the ethanol is produced at an instantaneous productivity of about 5 g/L-day to about 12 g/L-day. In another embodiment, the ethanol is produced at an instantaneous productivity of about 7 g/L-day to about 10 g/L-day.

In another embodiment, the broth comprises phytate. In another embodiment, the broth comprises proline, glycine, histidine, and/or isoleucine. In another embodiment, the broth comprises a cellulosic or lignocellulosic material. In another embodiment, the broth comprises a cellulosic or lignocellulosic material, wherein the cellulosic or lignocellulosic material was not enzymatically treated with a sufficient quantity of enzymes to convert more than 15% of the cellulosic or lignocellulosic material to simple sugars within 24 hours.

In another aspect, a process is disclosed in accordance with a preferred embodiment of the present invention for making ethanol. The process comprises (1) inoculating a medium suitable for growth of Clostridium phytofermentans with a culture of Clostridium phytofermentans resulting in a broth of Clostridium phytofermentans, wherein the culture of Clostridium phytofermentans was previously used to produce ethanol.

In one embodiment of the above-described process, the process further comprises growing the broth of Clostridium phytofermentans under conditions suitable for producing ethanol, producing ethanol, and recovering a material comprising ethanol from the broth.

In another embodiment, the process further comprises growing the broth of Clostridium phytofermentans in an ethanol concentration greater than about 6 g/L. In another embodiment, the process further comprises growing the broth of Clostridium phytofermentans in an ethanol concentration of about 6 to about 180 g/L. In another embodiment, the process further comprises growing the broth of Clostridium phytofermentans in an ethanol concentration of about 15 to about 160 g/L. In another embodiment, the process further comprises growing the broth of Clostridium phytofermentans in an ethanol concentration of about 20 to about 100 g/L. In another embodiment, the process further comprises growing the broth of Clostridium phytofermentans in an ethanol concentration of about 30 to about 80 g/L. In another embodiment, process further comprises growing the broth of Clostridium phytofermentans in an ethanol concentration of about 8 to about 14 g/L. In another embodiment, the process further comprises growing the broth of Clostridium phytofermentans under conditions suitable for producing ethanol, producing ethanol, and recovering a material comprising ethanol from the broth.

In another aspect, a process is disclosed in accordance with a preferred embodiment of the present invention for making ethanol. The process comprises (1) inoculating a volume of medium suitable for growth of Clostridium phytofermentans, with a volume of culture of Clostridium phytofermentans resulting in a broth of Clostridium phytofermentans; a ratio of the volume of culture to the culture of medium being greater than about 0.1 to about 1; and (2) growing the broth of Clostridium phytofermentans under conditions suitable for producing ethanol, and recovering a material comprising ethanol from the broth of Clostridium phytofermentans.

In one embodiment of the above described process, the ethanol is present while growing the broth at a concentration of about 8 to about 150 g/L. In another embodiment, the ratio of the volume of culture to the culture of medium is about 0.2 to about 1. In another embodiment, the ethanol is present while growing the broth at a concentration greater than about 8 g/L.

another methods and compositions for the production of a fuel are provided. In one aspect the inventions provides methods for producing alcohol. In some embodiments, the methods comprise fermenting cells of Clostridium phytofermentans in the presence of an added pH modifier, where an alcohol is produced. In some embodiments, the alcohol is ethanol.

In some embodiments of this aspect, fermentation of the cells occurs at a pH, where the pH is about 6.0 to about 7.2. In other embodiments, fermentation of the cells occurs at a pH, where the pH is about 6.2 to about 6.8.

In some embodiments of this aspect, the alcohol is produced at a concentration of about 15 to about 200 g/L. In other embodiments, the alcohol is produced at a concentration of about 15 to about 150 g/L. In other embodiments, the alcohol is produced at a concentration of about 18 to about 100 g/L. In other embodiments, the alcohol is produced at a concentration of about 20 to about 60 g/L.

In another aspect the invention provides methods for producing alcohol by fermenting cells of Clostridium phytofermentans in the presence of an added fatty acid comprising material, where an alcohol is produced. In some embodiments, the fatty acid comprising material is an edible fat or oil. In some embodiments, the fatty acid comprising material comprises a fatty acid with an unsaturation at the delta-9 position. In some embodiments, the fatty acid comprising material comprises a fatty acid with an unsaturation at the omega-9 position. In some embodiments, the fatty acid comprising material comprises one or more of oleic acid and linoleic acid. In some embodiments, the fatty acid comprising material comprises one or more of corn oil, sunflower oil, safflower oil, canola oil, soybean oil, or rape seed oil. In some embodiments, the fatty acid comprising material comprises a phospholipid or a lysophospholipid.

In another aspect the invention provides a fermentation broth, the broth comprising cells of Clostridium phytofermentans and an added pH modifier, where an alcohol is produced.

In another aspect the invention provides a fermentation broth, the broth comprising cells of a Clostridium phytofermentans and an added fatty acid containing compound, where an alcohol is produced.

In another aspect the invention provides methods of producing alcohol comprising fermenting cells of Clostridium phytofermentans and the presence of a pH modifier and a fatty acid comprising material, where alcohol is produced.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a graph of the substrate and ethanol concentrations of a batch fermentation with Clostridium phytofermentans.

FIG. 2 is a graph of the substrate and ethanol concentrations of fed-batch fermentations with Clostridium phytofermentans.

FIG. 3 is a graph of the ethanol concentration as a function of time during the fermentation of Clostridium phytofermentans with yeast extract.

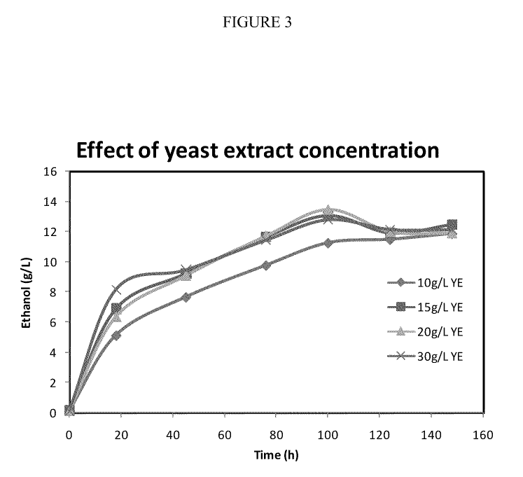

FIG. 4 shows a graph of ethanol concentration over time for fermentation conditions of different fatty acids.

FIG. 5 shows a graph of ethanol concentration over time for different fermentation conditions of pH.

FIG. 6 shows a graph of ethanol concentration over time for different fermentation conditions of fatty acid and pH.

FIG. 7 is a map of the plasmid pIMPT1029 used to transform Clostridium phytofermentans.

FIG. 8 is an example of a method for producing fermentive end products from biomass by first treating biomass with an acid at elevated temperature and pressure in a hydrolysis unit.

FIG. 9 depicts a method for producing fermentive end products from biomass by charging biomass to a fermentation vessel.

### DETAILED DESCRIPTION OF THE PREFERRED EMBODIMENT

### Definitions

Unless characterized otherwise, technical and scientific terms used herein have the same meaning as commonly understood by one of ordinary skill in the art to which this invention belongs.

“About” means a referenced numeric indication plus or minus 10% of that referenced numeric indication. For example the term about 4 would include a range of 3.6 to 4.4.

“Fermentive end-product” is used herein to include biofuels, chemicals, compounds suitable as liquid fuels, gaseous fuels, reagents, chemical feedstocks, chemical additives, processing aids, food additives, and other products. Examples of fermentive end-products include but are not limited to 1,4 diacids (succinic, fumaric and malic), 2,5 furan dicarboxylic acid, 3 hydroxy propionic acid, aspartic acid, glucaric acid, glutamic acid, itaconic acid, levulinic acid, 3-hydroxybutyrolactone, glycerol, sorbitol, xylitol/arabinitol, butanediol, butanol, methane, methanol, ethane, ethene, ethanol, n-propane, 1-propene, 1-propanol, propanal, acetone, propionate, n-butane, 1-butene, 1-butanol, butanal, butanoate, isobutanal, isobutanol, 2-methylbutanal, 2-methylbutanol, 3-methylbutanal, 3-methylbutanol, 2-butene, 2-butanol, 2-butanone, 2,3-butanediol, 3-hydroxy-2-butanone, 2,3-butanedione, ethylbenzene, ethenylbenzene, 2-phenylethanol, phenylacetaldehyde, 1-phenylbutane, 4-phenyl-1-butene, 4-phenyl-2-butene, 1-phenyl-2-butene, 1-phenyl-2-butanol, 4-phenyl-2-butanol, 1-phenyl-2-butanone, 4-phenyl-2-butanone, 1-phenyl-2,3-butandiol, 1-phenyl-3-hydroxy-2-butanone, 4-phenyl-3-hydroxy-2-butanone, 1-phenyl-2,3-butanedione, n-pentane, ethylphenol, ethenylphenol, 2-(4-hydroxyphenyl)ethanol, 4-hydroxyphenylacetaldehyde, 1-(4-hydroxyphenyl)butane, 4-(4-hydroxyphenyl)-1-butene, 4-(4-hydroxyphenyl)-2-butene, 1-(4-hydroxyphenyl)-1-butene, 1-(4-hydroxyphenyl)-2-butanol, 4-(4-hydroxyphenyl)-2-butanol, 1-(4-hydroxyphenyl)-2-butanone, 4-(4-hydroxyphenyl)-2-butanone, 1-(4-hydroxyphenyl)-2,3-butandiol, 1-(4-hydroxyphenyl)-3-hydroxy-2-butanone, 4-(4-hydroxyphenyl)-3-hydroxy-2-butanone, 1-(4-hydroxyphenyl)-2,3-butanonedione, indolylethane, indolylethene, 2-(indole-3-)ethanol, n-pentane, 1-pentene, 1-pentanol, pentanal, pentanoate, 2-pentene, 2-pentanol, 3-pentanol, 2-pentanone, 3-pentanone, 4-methylpentanal, 4-methylpentanol, 2,3-pentanediol, 2-hydroxy-3-pentanone, 3-hydroxy-2-pentanone, 2,3-pentanedione, 2-methylpentane, 4-methyl-1-pentene, 4-methyl-2-pentene, 4-methyl-3-pentene, 4-methyl-2-pentanol, 2-methyl-3-pentanol, 4-methyl-2-pentanone, 2-methyl-3-pentanone, 4-methyl-2,3-pentanediol, 4-methyl-2-hydroxy-3-pentanone, 4-methyl-3-hydroxy-2-pentanone, 4-methyl-2,3-pentanedione, 1-phenylpentane, 1-phenyl-1-pentene, 1-phenyl-2-pentene, 1-phenyl-3-pentene, 1-phenyl-2-pentanol, 1-phenyl-3-pentanol, 1-phenyl-2-pentanone, 1-phenyl-3-pentanone, 1-phenyl-2,3-pentanediol, 1-phenyl-2-hydroxy-3-pentanone, 1-phenyl-3-hydroxy-2-pentanone, 1-phenyl-2,3-pentanedione, 4-methyl-1-phenylpentane, 4-methyl-1-phenyl-1-pentene, 4-methyl-1-phenyl-2-pentene, 4-methyl-1-phenyl-3-pentene, 4-methyl-1-phenyl-3-pentanol, 4-methyl-1-phenyl-2-pentanol, 4-methyl-1-phenyl-3-pentanone, 4-methyl-1-phenyl-2-pentanone, 4-methyl-1-phenyl-2,3-pentanediol, 4-methyl-1-phenyl-2,3-pentanedione, 4-methyl-1-phenyl-3-hydroxy-2-pentanone, 4-methyl-1-phenyl-2-hydroxy-3-pentanone, 1-(4-hydroxyphenyl) pentane, 1-(4-hydroxyphenyl)-1-pentene, 1-(4-hydroxyphenyl)-2-pentene, 1-(4-hydroxyphenyl)-3-pentene, 1-(4-hydroxyphenyl)-2-pentanol, 1-(4-hydroxyphenyl)-3-pentanol, 1-(4-hydroxyphenyl)-2-pentanone, 1-(4-hydroxyphenyl)-3-pentanone, 1-(4-hydroxyphenyl)-2,3-pentanediol, 1-(4-hydroxyphenyl)-2-hydroxy-3-pentanone, 1-(4-hydroxyphenyl)-3-hydroxy-2-pentanone, 1-(4-hydroxyphenyl)-2,3-pentanedione, 4-methyl-1-(4-hydroxyphenyl)pentane, 4-methyl-1-(4-hydroxyphenyl)-2-pentene, 4-methyl-1-(4-hydroxyphenyl)-3-pentene, 4-methyl-1-(4-hydroxyphenyl)-1-pentene, 4-methyl-1-(4-hydroxyphenyl)-3-pentanol, 4-methyl-1-(4-hydroxyphenyl)-2-pentanol, 4-methyl-1-(4-hydroxyphenyl)-3-pentanone, 4-methyl-1-(4-hydroxyphenyl)-2-pentanone, 4-methyl-1-(4-hydroxyphenyl)-2,3-pentanediol, 4-methyl-1-(4-hydroxyphenyl)-2,3-pentanedione, 4-methyl-1-(4-hydroxyphenyl)-3-hydroxy-2-pentanone, 4-methyl-1-(4-hydroxyphenyl)-2-hydroxy-3-pentanone, 1-indole-3-pentane, 1-(indole-3)-1-pentene, 1-(indole-3)-2-pentene, 1-(indole-3)-3-pentene, 1-(indole-3)-2-pentanol, 1-(indole-3)-3-pentanol, 1-(indole-3)-2-pentanone, 1-(indole-3)-3-pentanone, 1-(indole-3)-2,3-pentanediol, 1-(indole-3)-2-hydroxy-3-pentanone, 1-(indole-3)-3-hydroxy-2-pentanone, 1-(indole-3)-2,3-pentanedione, 4-methyl-1-(indole-3-)pentane, 4-methyl-1-(indole-3)-2-pentene, 4-methyl-1-(indole-3)-3-pentene, 4-methyl-1-(indole-3)-1-pentene, 4-methyl-2-(indole-3)-3-pentanol, 4-methyl-1-(indole-3)-2-pentanol, 4-methyl-1-(indole-3)-3-pentanone, 4-methyl-1-(indole-3)-2-pentanone, 4-methyl-1-(indole-3)-2,3-pentanediol, 4-methyl-1-(indole-3)-2,3-pentanedione, 4-methyl-1-(indole-3)-3-hydroxy-2-pentanone, 4-methyl-1-(indole-3)-2-hydroxy-3-pentanone, n-hexane, 1-hexene, 1-hexanol, hexanal, hexanoate, 2-hexene, 3-hexene, 2-hexanol, 3-hexanol, 2-hexanone, 3-hexanone, 2,3-hexanediol, 2,3-hexanedione, 3,4-hexanediol, 3,4-hexanedione, 2-hydroxy-3-hexanone, 3-hydroxy-2-hexanone, 3-hydroxy-4-hexanone, 4-hydroxy-3-hexanone, 2-methylhexane, 3-methylhexane, 2-methyl-2-hexene, 2-methyl-3-hexene, 5-methyl-1-hexene, 5-methyl-2-hexene, 4-methyl-1-hexene, 4-methyl-2-hexene, 3-methyl-3-hexene, 3-methyl-2-hexene, 3-methyl-1-hexene, 2-methyl-3-hexanol, 5-methyl-2-hexanol, 5-methyl-3-hexanol, 2-methyl-3-hexanone, 5-methyl-2-hexanone, 5-methyl-3-hexanone, 2-methyl-3,4-hexanediol, 2-methyl-3,4-hexanedione, 5-methyl-2,3-hexanediol, 5-methyl-2,3-hexanedione, 4-methyl-2,3-hexanediol, 4-methyl-2,3-hexanedione, 2-methyl-3-hydroxy-4-hexanone, 2-methyl-4-hydroxy-3-hexanone, 5-methyl-2-hydroxy-3-hexanone, 5-methyl-3-hydroxy-2-hexanone, 4-methyl-2-hydroxy-3-hexanone, 4-methyl-3-hydroxy-2-hexanone, 2,5-dimethylhexane, 2,5-dimethyl-2-hexene, 2,5-dimethyl-3-hexene, 2,5-dimethyl-3-hexanol, 2,5-dimethyl-3-hexanone, 2,5-dimethyl-3,4-hexanediol, 2,5-dimethyl-3,4-hexanedione, 2,5-dimethyl-3-hydroxy-4-hexanone, 5-methyl-1-phenylhexane, 4-methyl-1-phenylhexane, 5-methyl-1-phenyl-1-hexene, 5-methyl-1-phenyl-2-hexene, 5-methyl-1-phenyl-3-hexene, 4-methyl-1-phenyl-1-hexene, 4-methyl-1-phenyl-2-hexene, 4-methyl-1-phenyl-3-hexene, 5-methyl-1-phenyl-2-hexanol, 5-methyl-1-phenyl-3-hexanol, 4-methyl-1-phenyl-2-hexanol, 4-methyl-1-phenyl-3-hexanol, 5-methyl-1-phenyl-2-hexanone, 5-methyl-1-phenyl-3-hexanone, 4-methyl-1-phenyl-2-hexanone, 4-methyl-1-phenyl-3-hexanone, 5-methyl-1-phenyl-2,3-hexanediol, 4-methyl-1-phenyl-2,3-hexanediol, 5-methyl-1-phenyl-3-hydroxy-2-hexanone, 5-methyl-1-phenyl-2-hydroxy-3-hexanone, 4-methyl-1-phenyl-3-hydroxy-2-hexanone, 4-methyl-1-phenyl-2-hydroxy-3-hexanone, 5-methyl-1-phenyl-2,3-hexanedione, 4-methyl-1-phenyl-2,3-hexanedione, 4-methyl-1-(4-hydroxyphenyl)hexane, 5-methyl-1-(4-hydroxyphenyl)-1-hexene, 5-methyl-1-(4-hydroxyphenyl)-2-hexene, 5-methyl-1-(4-hydroxyphenyl)-3-hexene, 4-methyl-1-(4-hydroxyphenyl)-1-hexene, 4-methyl-1-(4-hydroxyphenyl)-2-hexene, 4-methyl-1-(4-hydroxyphenyl)-3-hexene, 5-methyl-1-(4-hydroxyphenyl)-2-hexanol, 5-methyl-1-(4-hydroxyphenyl)-3-hexanol, 4-methyl-1-(4-hydroxyphenyl)-2-hexanol, 4-methyl-1-(4-hydroxyphenyl)-3-hexanol, 5-methyl-1-(4-hydroxyphenyl)-2-hexanone, 5-methyl-1-(4-hydroxyphenyl)-3-hexanone, 4-methyl-1-(4-hydroxyphenyl)-2-hexanone, 4-methyl-1-(4-hydroxyphenyl)-3-hexanone, 5-methyl-1-(4-hydroxyphenyl)-2,3-hexanediol, 4-methyl-1-(4-hydroxyphenyl)-2,3-hexanediol, 5-methyl-1-(4-hydroxyphenyl)-3-hydroxy-2-hexanone, 5-methyl-1-(4-hydroxyphenyl)-2-hydroxy-3-hexanone, 4-methyl-1-(4-hydroxyphenyl)-3-hydroxy-2-hexanone, 4-methyl-1-(4-hydroxyphenyl)-2-hydroxy-3-hexanone, 5-methyl-1-(4-hydroxyphenyl)-2,3-hexanedione, 4-methyl-1-(4-hydroxyphenyl)-2,3-hexanedione, 4-methyl-1-(indole-3-)hexane, 5-methyl-1-(indole-3)-1-hexene, 5-methyl-1-(indole-3)-2-hexene, 5-methyl-1-(indole-3)-3-hexene, 4-methyl-1-(indole-3)-1-hexene, 4-methyl-1-(indole-3)-2-hexene, 4-methyl-1-(indole-3)-3-hexene, 5-methyl-1-(indole-3)-2-hexanol, 5-methyl-1-(indole-3)-3-hexanol, 4-methyl-1-(indole-3)-2-hexanol, 4-methyl-1-(indole-3)-3-hexanol, 5-methyl-1-(indole-3)-2-hexanone, 5-methyl-1-(indole-3)-3-hexanone, 4-methyl-1-(indole-3)-2-hexanone, 4-methyl-1-(indole-3)-3-hexanone, 5-methyl-1-(indole-3)-2,3-hexanediol, 4-methyl-1-(indole-3)-2,3-hexanediol, 5-methyl-1-(indole-3)-3-hydroxy-2-hexanone, 5-methyl-1-(indole-3)-2-hydroxy-3-hexanone, 4-methyl-1-(indole-3)-3-hydroxy-2-hexanone, 4-methyl-1-(indole-3)-2-hydroxy-3-hexanone, 5-methyl-1-(indole-3)-2,3-hexanedione, 4-methyl-1-(indole-3)-2,3-hexanedione, n-heptane, 1-heptene, 1-heptanol, heptanal, heptanoate, 2-heptene, 3-heptene, 2-heptanol, 3-heptanol, 4-heptanol, 2-heptanone, 3-heptanone, 4-heptanone, 2,3-heptanediol, 2,3-heptanedione, 3,4-heptanediol, 3,4-heptanedione, 2-hydroxy-3-heptanone, 3-hydroxy-2-heptanone, 3-hydroxy-4-heptanone, 4-hydroxy-3-heptanone, 2-methylheptane, 3-methylheptane, 6-methyl-2-heptene, 6-methyl-3-heptene, 2-methyl-3-heptene, 2-methyl-2-heptene, 5-methyl-2-heptene, 5-methyl-3-heptene, 3-methyl-3-heptene, 2-methyl-3-heptanol, 2-methyl-4-heptanol, 6-methyl-3-heptanol, 5-methyl-3-heptanol, 3-methyl-4-heptanol, 2-methyl-3-heptanone, 2-methyl-4-heptanone, 6-methyl-3-heptanone, 5-methyl-3-heptanone, 3-methyl-4-heptanone, 2-methyl-3,4-heptanediol, 2-methyl-3,4-heptanedione, 6-methyl-3,4-heptanediol, 6-methyl-3,4-heptanedione, 5-methyl-3,4-heptanediol, 5-methyl-3,4-heptanedione, 2-methyl-3-hydroxy-4-heptanone, 2-methyl-4-hydroxy-3-heptanone, 6-methyl-3-hydroxy-4-heptanone, 6-methyl-4-hydroxy-3-heptanone, 5-methyl-3-hydroxy-4-heptanone, 5-methyl-4-hydroxy-3-heptanone, 2,6-dimethylheptane, 2,5-dimethylheptane, 2,6-dimethyl-2-heptene, 2,6-dimethyl-3-heptene, 2,5-dimethyl-2-heptene, 2,5-dimethyl-3-heptene, 3,6-dimethyl-3-heptene, 2,6-dimethyl-3-heptanol, 2,6-dimethyl-4-heptanol, 2,5-dimethyl-3-heptanol, 2,5-dimethyl-4-heptanol, 2,6-dimethyl-3,4-heptanediol, 2,6-dimethyl-3,4-heptanedione, 2,5-dimethyl-3,4-heptanediol, 2,5-dimethyl-3,4-heptanedione, 2,6-dimethyl-3-hydroxy-4-heptanone, 2,6-dimethyl-4-hydroxy-3-heptanone, 2,5-dimethyl-3-hydroxy-4-heptanone, 2,5-dimethyl-4-hydroxy-3-heptanone, n-octane, 1-octene, 2-octene, 1-octanol, octanal, octanoate, 3-octene, 4-octene, 4-octanol, 4-octanone, 4,5-octanediol, 4,5-octanedione, 4-hydroxy-5-octanone, 2-methyloctane, 2-methyl-3-octene, 2-methyl-4-octene, 7-methyl-3-octene, 3-methyl-3-octene, 3-methyl-4-octene, 6-methyl-3-octene, 2-methyl-4-octanol, 7-methyl-4-octanol, 3-methyl-4-octanol, 6-methyl-4-octanol, 2-methyl-4-octanone, 7-methyl-4-octanone, 3-methyl-4-octanone, 6-methyl-4-octanone, 2-methyl-4,5-octanediol, 2-methyl-4,5-octanedione, 3-methyl-4,5-octanediol, 3-methyl-4,5-octanedione, 2-methyl-4-hydroxy-5-octanone, 2-methyl-5-hydroxy-4-octanone, 3-methyl-4-hydroxy-5-octanone, 3-methyl-5-hydroxy-4-octanone, 2,7-dimethyloctane, 2,7-dimethyl-3-octene, 2,7-dimethyl-4-octene, 2,7-dimethyl-4-octanol, 2,7-dimethyl-4-octanone, 2,7-dimethyl-4,5-octanediol, 2,7-dimethyl-4,5-octanedione, 2,7-dimethyl-4-hydroxy-5-octanone, 2,6-dimethyloctane, 2,6-dimethyl-3-octene, 2,6-dimethyl-4-octene, 3,7-dimethyl-3-octene, 2,6-dimethyl-4-octanol, 3,7-dimethyl-4-octanol, 2,6-dimethyl-4-octanone, 3,7-dimethyl-4-octanone, 2,6-dimethyl-4,5-octanediol, 2,6-dimethyl-4,5-octanedione, 2,6-dimethyl-4-hydroxy-5-octanone, 2,6-dimethyl-5-hydroxy-4-octanone, 3,6-dimethyloctane, 3,6-dimethyl-3-octene, 3,6-dimethyl-4-octene, 3,6-dimethyl-4-octanol, 3,6-dimethyl-4-octanone, 3,6-dimethyl-4,5-octanediol, 3,6-dimethyl-4,5-octanedione, 3,6-dimethyl-4-hydroxy-5-octanone, n-nonane, 1-nonene, 1-nonanol, nonanal, nonanoate, 2-methylnonane, 2-methyl-4-nonene, 2-methyl-5-nonene, 8-methyl-4-nonene, 2-methyl-5-nonanol, 8-methyl-4-nonanol, 2-methyl-5-nonanone, 8-methyl-4-nonanone, 8-methyl-4,5-nonanediol, 8-methyl-4,5-nonanedione, 8-methyl-4-hydroxy-5-nonanone, 8-methyl-5-hydroxy-4-nonanone, 2,8-dimethylnonane, 2,8-dimethyl-3-nonene, 2,8-dimethyl-4-nonene, 2,8-dimethyl-5-nonene, 2,8-dimethyl-4-nonanol, 2,8-dimethyl-5-nonanol, 2,8-dimethyl-4-nonanone, 2,8-dimethyl-5-nonanone, 2,8-dimethyl-4,5-nonanediol, 2,8-dimethyl-4,5-nonanedione, 2,8-dimethyl-4-hydroxy-5-nonanone, 2,8-dimethyl-5-hydroxy-4-nonanone, 2,7-dimethylnonane, 3,8-dimethyl-3-nonene, 3,8-dimethyl-4-nonene, 3,8-dimethyl-5-nonene, 3,8-dimethyl-4-nonanol, 3,8-dimethyl-5-nonanol, 3,8-dimethyl-4-nonanone, 3,8-dimethyl-5-nonanone, 3,8-dimethyl-4,5-nonanediol, 3,8-dimethyl-4,5-nonanedione, 3,8-dimethyl-4-hydroxy-5-nonanone, 3,8-dimethyl-5-hydroxy-4-nonanone, n-decane, 1-decene, 1-decanol, decanoate, 2,9-dimethyldecane, 2,9-dimethyl-3-decene, 2,9-dimethyl-4-decene, 2,9-dimethyl-5-decanol, 2,9-dimethyl-5-decanone, 2,9-dimethyl-5,6-decanediol, 2,9-dimethyl-6-hydroxy-5-decanone, 2,9-dimethyl-5,6-decanedionen-undecane, 1-undecene, 1-undecanol, undecanal. undecanoate, n-dodecane, 1-dodecene, 1-dodecanol, dodecanal, dodecanoate, n-dodecane, 1-decadecene, 1-dodecanol, dodecanal, dodecanoate, n-tridecane, 1-tridecene, 1-tridecanol, tridecanal, tridecanoate, n-tetradecane, 1-tetradecene, 1-tetradecanol, tetradecanal, tetradecanoate, n-pentadecane, 1-pentadecene, 1-pentadecanol, pentadecanal, pentadecanoate, n-hexadecane, 1-hexadecene, 1-hexadecanol, hexadecanal, hexadecanoate, n-heptadecane, 1-heptadecene, 1-heptadecanol, heptadecanal, heptadecanoate, n-octadecane, 1-octadecene, 1-octadecanol, octadecanal, octadecanoate, n-nonadecane, 1-nonadecene, 1-nonadecanol, nonadecanal, nonadecanoate, eicosane, 1-eicosene, 1-eicosanol, eicosanal, eicosanoate, 3-hydroxy propanal, 1,3-propanediol, 4-hydroxybutanal, 1,4-butanediol, 3-hydroxy-2-butanone, 2,3-butandiol, 1,5-pentane diol, homocitrate, homoisocitorate, b-hydroxy adipate, glutarate, glutarsemialdehyde, glutaraldehyde, 2-hydroxy-1-cyclopentanone, 1,2-cyclopentanediol, cyclopentanone, cyclopentanol, (S)-2-acetolactate, (R)-2,3-Dihydroxy-isovalerate, 2-oxoisovalerate, isobutyryl-CoA, isobutyrate, isobutyraldehyde, 5-amino pentaldehyde, 1,10-diaminodecane, 1,10-diamino-5-decene, 1,10-diamino-5-hydroxydecane, 1,10-diamino-5-decanone, 1,10-diamino-5,6-decanediol, 1,10-diamino-6-hydroxy-5-decanone, phenylacetoaldehyde, 1,4-diphenylbutane, 1,4-diphenyl-1-butene, 1,4-diphenyl-2-butene, 1,4-diphenyl-2-butanol, 1,4-diphenyl-2-butanone, 1,4-diphenyl-2,3-butanediol, 1,4-diphenyl-3-hydroxy-2-butanone, 1-(4-hydeoxyphenyl)-4-phenylbutane, 1-(4-hydeoxyphenyl)-4-phenyl-1-butene, 1-(4-hydeoxyphenyl)-4-phenyl-2-butene, 1-(4-hydeoxyphenyl)-4-phenyl-2-butanol, 1-(4-hydeoxyphenyl)-4-phenyl-2-butanone, 1-(4-hydeoxyphenyl)-4-phenyl-2,3-butanediol, 1-(4-hydeoxyphenyl)-4-phenyl-3-hydroxy-2-butanone, 1-(indole-3)-4-phenylbutane, 1-(indole-3)-4-phenyl-1-butene, 1-(indole-3)-4-phenyl-2-butene, 1-(indole-3)-4-phenyl-2-butanol, 1-(indole-3)-4-phenyl-2-butanone, 1-(indole-3)-4-phenyl-2,3-butanediol, 1-(indole-3)-4-phenyl-3-hydroxy-2-butanone, 4-hydroxyphenylacetoaldehyde, 1,4-di(4-hydroxyphenyl)butane, 1,4-di(4-hydroxyphenyl)-1-butene, 1,4-di(4-hydroxyphenyl)-2-butene, 1,4-di(4-hydroxyphenyl)-2-butanol, 1,4-di(4-hydroxyphenyl)-2-butanone, 1,4-di(4-hydroxyphenyl)-2,3-butanediol, 1,4-di(4-hydroxyphenyl)-3-hydroxy-2-butanone, 1-(4-hydroxyphenyl)-4-(indole-3-)butane, 1-(4-hydroxyphenyl)-4-(indole-3)-1-butene, 1-di(4-hydroxyphenyl)-4-(indole-3)-2-butene, 1-(4-hydroxyphenyl)-4-(indole-3)-2-butanol, 1-(4-hydroxyphenyl)-4-(indole-3)-2-butanone, 1-(4-hydroxyphenyl)-4-(indole-3)-2,3-butanediol, 1-(4-hydroxyphenyl-4-(indole-3)-3-hydroxy-2-butanone, indole-3-acetoaldehyde, 1,4-di(indole-3-) butane, 1,4-di(indole-3)-1-butene, 1,4-di(indole-3)-2-butene, 1,4-di(indole-3)-2-butanol, 1,4-di(indole-3)-2-butanone, 1,4-di(indole-3)-2,3-butanediol, 1,4-di(indole-3)-3-hydroxy-2-butanone, succinate semialdehyde, hexane-1,8-dicarboxylic acid, 3-hexene-1,8-dicarboxylic acid, 3-hydroxy-hexane-1,8-dicarboxylic acid, 3-hexanone-1,8-dicarboxylic acid, 3,4-hexanediol-1,8-dicarboxylic acid, 4-hydroxy-3-hexanone-1,8-dicarboxylic acid, fucoidan, iodine, chlorophyll, carotenoid, calcium, magnesium, iron, sodium, potassium, phosphate, lactic acid, acetic acid, formic acid, isoprenoids, and polyisoprenes, including rubber. Further, such products can include succinic acid, pyruvic acid, enzymes such as cellulases, polysaccharases, lipases, proteases, ligninases, and hemicellulases and may be present as a pure compound, a mixture, or an impure or diluted form.

The term “fatty acid comprising material” as used herein has its ordinary meaning as known to those skilled in the art and can comprise one or more chemical compounds that include one or more fatty acid moieties as well as derivatives of these compounds and materials that comprise one or more of these compounds. Common examples of compounds that include one or more fatty acid moieties include triacylglycerides, diacylglycerides, monoacylglycerides, phospholipids, lysophospholipids, free fatty acids, fatty acid salts, soaps, fatty acid comprising amides, esters of fatty acids and monohydric alcohols, esters of fatty acids and polyhydric alcohols including glycols (e.g. ethylene glycol, propylene glycol, etc.), esters of fatty acids and polyethylene glycol, esters of fatty acids and polyethers, esters of fatty acids and polyglycol, esters of fatty acids and saccharides, esters of fatty acids with other hydroxyl-containing compounds, etc. A fatty acid comprising material can be one or more of these compounds in an isolated or purified form. It can be a material that includes one or more of these compounds that is combined or blended with other similar or different materials. It can be a material where the fatty acid comprising material occurs with or is provided with other similar or different materials, such as vegetable and animal oils; mixtures of vegetable and animal oils; vegetable and animal oil byproducts; mixtures of vegetable and animal oil byproducts; vegetable and animal wax esters; mixtures, derivatives and byproducts of vegetable and animal wax esters; seeds; processed seeds; seed byproducts; nuts; processed nuts; nut byproducts; animal matter; processed animal matter; byproducts of animal matter; corn; processed corn; corn byproducts; distiller's grains; beans; processed beans; bean byproducts; soy products; lipid containing plant, fish or animal matter; processed lipid containing plant or animal matter; byproducts of lipid containing plant, fish or animal matter; lipid containing microbial material; processed lipid containing microbial material; and byproducts of lipid containing microbial matter. Such materials can be utilized in liquid or solid forms. Solid forms include whole forms, such as cells, beans, and seeds; ground, chopped, slurried, extracted, flaked, milled, etc. The fatty acid portion of the fatty acid comprising compound can be a simple fatty acid, such as one that includes a carboxyl group attached to a substituted or un-substituted alkyl group. The substituted or unsubstituted alkyl group can be straight or branched, saturated or unsaturated. Substitutions on the alkyl group can include hydroxyls, phosphates, halogens, alkoxy, or aryl groups. The substituted or unsubstituted alkyl group can have 7 to 29 carbons and preferably 11 to 23 carbons (e.g., 8 to 30 carbons and preferably 12 to 24 carbons counting the carboxyl group) arranged in a linear chain with or without side chains and/or substitutions. Addition of the fatty acid comprising compound can be by way of adding a material comprising the fatty acid comprising compound.

The term “pH modifier” as used herein has its ordinary meaning as known to those skilled in the art and can include any material that will tend to increase, decrease or hold steady the pH of the broth or medium. A pH modifier can be an acid, a base, a buffer, or a material that reacts with other materials present to serve to raise, lower, or hold steady the pH. In some embodiments, more than one pH modifier can be used, such as more than one acid, more than one base, one or more acid with one or more bases, one or more acids with one or more buffers, one or more bases with one or more buffers, or one or more acids with one or more bases with one or more buffers. In some embodiments, a buffer can be produced in the broth or medium or separately and used as an ingredient by at least partially reacting in acid or base with a base or an acid, respectively. When more than one pH modifiers are utilized, they can be added at the same time or at different times. In some embodiments, one or more acids and one or more bases can be combined, resulting in a buffer. In some embodiments, media components, such as a carbon source or a nitrogen source can also serve as a pH modifier; suitable media components include those with high or low pH or those with buffering capacity. Exemplary media components include acid- or base-hydrolyzed plant polysaccharides having residual acid or base, ammonia fiber explosion (AFEX) treated plant material with residual ammonia, lactic acid, corn steep solids or liquor.

The term “fermentation” as used herein has its ordinary meaning as known to those skilled in the art and can include culturing of a microorganism or group of microorganisms in or on a suitable medium for the microorganisms. The microorganisms can be aerobes, anaerobes, facultative anaerobes, heterotrophs, autotrophs, photoautotrophs, photoheterotrophs, chemoautotrophs, and/or chemoheterotrophs. The microorganisms can be growing aerobically or anaerobically. They can be in any phase of growth, including lag (or conduction), exponential, transition, stationary, death, dormant, vegetative, sporulating, etc.

“Growth phase” is used herein to describe the type of cellular growth that occurs after the “Initiation phase” and before the “Stationary phase” and the “Death phase.” The growth phase is sometimes referred to as the exponential phase or log phase or logarithmic phase.

The term “plant polysaccharide” as used herein has its ordinary meaning as known to those skilled in the art and can comprise one or more polymers of sugars and sugar derivatives as well as derivatives of sugar polymers and/or other polymeric materials that occur in plant matter. Exemplary plant polysaccharides include lignin, cellulose, starch, pectin, and hemicellulose. Others are chitin, sulfonated polysaccharides such as alginic acid, agarose, carrageenan, porphyran, furcelleran and funoran. Generally, the polysaccharide can have two or more sugar units or derivatives of sugar units. The sugar units and/or derivatives of sugar units can repeat in a regular pattern, or otherwise. The sugar units can be hexose units or pentose units, or combinations of these. The derivatives of sugar units can be sugar alcohols, sugar acids, amino sugars, etc. The polysaccharides can be linear, branched, cross-linked, or a mixture thereof. One type or class of polysaccharide can be cross-linked to another type or class of polysaccharide.

The term “fermentable sugars” as used herein has its ordinary meaning as known to those skilled in the art and can include one or more sugars and/or sugar derivatives that can be utilized as a carbon source by the microorganism, including monomers, dimers, and polymers of these compounds including two or more of these compounds. In some cases, the organism can break down these polymers, such as by hydrolysis, prior to incorporating the broken down material. Exemplary fermentable sugars include, but are not limited to glucose, xylose, arabinose, galactose, mannose, rhamnose, cellobiose, lactose, sucrose, maltose, and fructose.

The term “saccharification” as used herein has its ordinary meaning as known to those skilled in the art and can include conversion of plant polysaccharides to lower molecular weight species that can be utilized by the organism at hand. For some organisms, this would include conversion to monosaccharides, disaccharides, trisaccharides, and oligosaccharides of up to about seven monomer units, as well as similar sized chains of sugar derivatives and combinations of sugars and sugar derivatives. For some organisms, the allowable chain-length can be longer and for some organisms the allowable chain-length can be shorter.

The term “biomass” as used herein has its ordinary meaning as known to those skilled in the art and can include one or more biological materials that can be converted into a biofuel, chemical or other product. One exemplary source of biomass is plant matter. Plant matter can be, for example, woody plant matter, non-woody plant matter, cellulosic material, lignocellulosic material, hemicellulosic material, carbohydrates, pectin, starch, inulin, fructans, glucans, corn, sugar cane, grasses, switchgrass, bamboo, algae and material derived from these. Plant matter can be further described by reference to the chemical species present, such as proteins, polysaccharides and oils. Polysaccharides include polymers of various monosaccharides and derivatives of monosaccharides including glucose, fructose, lactose, galacturonic acid, rhamnose, etc. Plant matter also includes agricultural waste byproducts or side streams such as pomace, corn steep liquor, corn steep solids, distillers grains, peels, pits, fermentation waste, straw, lumber, sewage, garbage and food leftovers. These materials can come from farms, forestry, industrial sources, households, etc. Another non-limiting example of biomass is animal matter, including, for example milk, meat, fat, animal processing waste, and animal waste. “Feedstock” is frequently used to refer to biomass being used for a process, such as those described herein.

“Broth” is used herein to refer to inoculated medium at any stage of growth, including the point immediately after inoculation and the period after any or all cellular activity has ceased and can include the material after post-fermentation processing. It includes the entire contents of the combination of soluble and insoluble matter, suspended matter, cells and medium, as appropriate.

The term “productivity” as used herein has its ordinary meaning as known to those skilled in the art and can include the mass of a material of interest produced in a given time in a given volume. Units can be, for example, grams per liter-hour, or some other combination of mass, volume, and time. In fermentation, productivity is frequently used to characterize how fast a product can be made within a given fermentation volume. The volume can be referenced to the total volume of the fermentation vessel, the working volume of the fermentation vessel, or the actual volume of broth being fermented. The context of the phrase will indicate the meaning intended to one of skill in the art. Productivity is different from “titer” in that productivity includes a time term, and titer is analogous to concentration. Titer and Productivity can generally be measured at any time during the fermentation, such as at the beginning, the end, or at some intermediate time, with titer relating the amount of a particular material present or produced at the point in time of interest and the productivity relating the amount of a particular material produced per liter in a given amount of time. The amount of time used in the productivity determination can be from the beginning of the fermentation or from some other time, and go to the end of the fermentation, such as when no additional material is produced or when harvest occurs, or some other time as indicated by the context of the use of the term. “Overall productivity” refers to the productivity determined by utilizing the final titer and the overall fermentation time. “Productivity to maximum titer” refers to the productivity determined utilizing the maximum titer and the time to achieve the maximum titer. “Instantaneous productivity” refers to the productivity at a moment in time and can be determined from the slope of the titer v. time curve for the compound of interest, or by other appropriate means as determined by the circumstances of the operation and the context of the language. “Incremental productivity” refers to productivity over a portion of the fermentation time, such as several minutes, an hour, or several hours. Frequently, an incremental productivity is used to imply or approximate instantaneous productivity. Other types of productivity can be used as well, with the context indicating how the value should be determined.

“Titer” refers to the amount of a particular material present in a fermentation broth. It is similar to concentration and can refer to the amount of material made by the organism in the broth from all fermentation cycles, or the amount of material made in the current fermentation cycle or over a given period of time, or the amount of material present from whatever source, such as produced by the organism or added to the broth. Frequently, the titer of soluble species will be referenced to the liquid portion of the broth, with insolubles removed, and the titer of insoluble species will be referenced to the total amount of broth with insoluble species being present, however, the titer of soluble species can be referenced to the total broth volume and the titer of insoluble species can be referenced to the liquid portion, with the context indicating the which system is used with both reference systems intended in some cases. Frequently, the value determined referenced to one system will be the same or a sufficient approximation of the value referenced to the other. “Concentration” when referring to material in the broth generally refers to the amount of a material present from all sources, whether made by the organism or added to the broth. Concentration can refer to soluble species or insoluble species, and is referenced to either the liquid portion of the broth or the total volume of the broth, as for “titer.”

The term “biocatalyst” as used herein has its ordinary meaning as known to those skilled in the art and can include one or more enzymes and microorganisms, including solutions, suspensions, and mixtures of enzymes and microorganisms. In some contexts this word will refer to the possible use of either enzymes or microorganisms to serve a particular function, in other contexts the word will refer to the combined use of the two, and in other contexts the word will refer to only one of the two. The context of the phrase will indicate the meaning intended to one of skill in the art.

The terms “conversion efficiency” or “yield” as used herein have their ordinary meaning as known to those skilled in the art and can include the mass of product made from a mass of substrate. The term can be expressed as a percentage yield of the product from a starting mass of substrate. For the production of ethanol from glucose, the net reaction is generally accepted as:

C₆H₁₂O₆→2C₂H₅OH+2 CO₂

and the theoretical maximum conversion efficiency, or yield, is 51% (wt.). Frequently, the conversion efficiency will be referenced to the theoretical maximum, for example, “80% of the theoretical maximum.” In the case of conversion of glucose to ethanol, this statement would indicate a conversion efficiency of 41% (wt.). The context of the phrase will indicate the substrate and product intended to one of skill in the art.

“Pretreatment” or “pretreated” is used herein to refer to any mechanical, chemical, thermal, biochemical process or combination of these processes whether in a combined step or performed sequentially, that achieves disruption or expansion of the biomass so as to render the biomass more susceptible to attack by enzymes and/or microbes. In some embodiments, pretreatment can include removal or disruption of lignin so as to make the cellulose and hemicellulose polymers in the plant biomass more available to cellulolytic enzymes and/or microbes, for example, by treatment with acid or base. In some embodiments, pretreatment can include the use of a microorganism of one type to render plant polysaccharides more accessible to microorganisms of another type, for example, by treatment with acid or base. In some embodiments, pretreatment can also include disruption or expansion of cellulosic and/or hemicellulosic material. Steam explosion, and ammonia fiber expansion (or explosion) (AFEX) are well known thermal/chemical techniques. Hydrolysis, including methods that utilize acids, bases, and/or enzymes can be used. Other thermal, chemical, biochemical, enzymatic techniques can also be used.

“Fed-batch” or “fed-batch fermentation” is used herein to include methods of culturing microorganisms where nutrients, other medium components, or biocatalysts (including, for example, enzymes, fresh organisms, extracellular broth, etc.) are supplied to the fermentor during cultivation, but culture broth is not harvested from the fermentor until the end of the fermentation, although it can also include “self seeding” or “partial harvest” techniques where a portion of the fermentor volume is harvested and then fresh medium is added to the remaining broth in the fermentor, with at least a portion of the inoculum being the broth that was left in the fermentor. During a fed-batch fermentation, the broth volume can increase, at least for a period, by adding medium or nutrients to the broth while fermentation organisms are present. In some fed-batch fermentations, the broth volume can be insensitive to the addition of nutrients and in some cases not change from the addition of nutrients. Suitable nutrients which can be utilized include those that are soluble, insoluble, and partially soluble, including gasses, liquids and solids. In some embodiments, a fed-batch process might be referred to with a phrase such as, “fed-batch with cell augmentation.” This phrase can include an operation where nutrients and cells are added or one where cells with no substantial amount of nutrients are added. The more general phrase “fed-batch” encompasses these operations as well. The context where any of these phrases is used will indicate to one of skill in the art the techniques being considered.

A term “phytate” as used herein has its ordinary meaning as known to those skilled in the art can be include phytic acid, its salts, and its combined forms as well as combinations of these.

“Sugar compounds” is used herein to include monosaccharide sugars, including but not limited to hexoses and pentoses; sugar alcohols; sugar acids; sugar amines; compounds containing two or more of these linked together directly or indirectly through covalent or ionic bonds; and mixtures thereof. Included within this description are disaccharides; trisaccharides; oligosaccharides; polysaccharides; and sugar chains, branched and/or linear, of any length.

“Dry cell weight” is used herein to refer to a method of determining the cell content of a broth or inoculum, and the value so determined. Generally, the method includes rinsing or washing a volume of broth followed by drying and weighing the residue, but is not necessary. In some cases, a sample of broth is simply centrifuged with the layer containing cells collected, dried, and weighed. Frequently, the broth is centrifuged, then resuspended in water or a mixture of water and other ingredients, such as a buffer, ingredients to create an isotonic condition, ingredients to control any change in osmotic pressure, etc. The centrifuge-resuspend steps can be repeated, if desired, and different resuspending solutions can be used prior to the final centrifuging and drying. When an insoluble medium component is present, the presence of the insoluble component can be ignored, with the value determined as above. Preferred methods when insoluble medium components are present include those where the insoluble component is reacted to a soluble form, dissolved or extracted into a different solvent that can include water, or separated by an appropriate method, such as by centrifugation, gradient centrifugation, flotation, filtration, or other suitable technique or combination of techniques.

### DESCRIPTION

The following description and examples illustrate some exemplary embodiments of the disclosed invention in detail. Those of skill in the art will recognize that there are numerous variations and modifications of this invention that are encompassed by its scope. Accordingly, the description of a certain exemplary embodiment should not be deemed to limit the scope of the present invention.

C. phytofermentans (“Q microbe”) includes American Type Culture Collection 700394^{T}, and can in some embodiments be defined based on the phenotypic and genotypic characteristics of a cultured strain, ISDg^{T} (Warnick et al., International Journal of Systematic and Evolutionary Microbiology, 52:1155-60, 2002). Aspects of the invention generally include systems, methods, and compositions for producing fuels, such as ethanol, and/or other useful organic products involving, for example, strain ISDg^{T} and/or any other strain of the species Clostridium phytofermentans, including those which can be derived from strain ISDg^{T}, including genetically modified strains, or strains separately isolated. Some exemplary species can be defined using standard taxonomic considerations (Stackebrandt and Goebel, International Journal of Systematic Bacteriology, 44:846-9, 1994): Strains with 16S rRNA sequence homology values of 97% and higher as compared to the type strain (ISDg^{T}), and strains with DNA re-association values of at least about 70% can be considered Clostridium phytofermentans. Considerable evidence exists to indicate that many microbes which have 70% or greater DNA re-association values also have at least 96% DNA sequence identity and share phenotypic traits defining a species. Analyses of the genome sequence of Clostridium phytofermentans strain ISDg^{T} indicate the presence of large numbers of genes and genetic loci that are likely to be involved in mechanisms and pathways for plant polysaccharide fermentation, giving rise to the unusual fermentation properties of this microbe which can be found in all or nearly all strains of the species Clostridium phytofermentans. Clostridium phytofermentans strains can be natural isolates, or genetically modified strains.

### Attributes of C. Phytofermentans

The “Q” microbe provides useful advantages for the conversion of biomass to ethanol and other products. One advantage of the Q microbe is its ability to produce enzymes capable of hydrolyzing polysaccharides and higher molecular weight saccharides to lower molecular weight saccharides, such as oligosaccharides, disaccharides, and monosaccharides. The Q microbe can produce a wide spectrum of hydrolytic enzymes, which can facilitate fermenting of various biomass materials, including cellulosic, hemicellulosic, lignocellulosic materials; pectins; starches; wood; paper; agricultural products; forest waste; tree waste; tree bark; leaves; grasses; sawgrass; woody plant matter; non-woody plant matter; carbohydrates; pectin; starch; inulin; fructans; glucans; corn; sugar cane; grasses; bamboo, algae, and material derived from these materials. The organism can usually produce these enzymes as needed, frequently without excessive production of unnecessary hydrolytic enzymes, or in some embodiments, one or more enzymes can be added to further improve the organism's production capability. This ability to produce a very wide range of hydrolytic enzymes gives the Q microbe and the associated technology distinct advantages in biomass fermentation, especially those fermentations not utilizing simple sugars as the feedstock. Various fermentation conditions can enhance the activities of the organism, resulting in higher yields, higher productivity, greater product selectivity, and/or greater conversion efficiency. In some embodiments, fermentation conditions can include fed batch operation and fed batch operation with cell augmentation; addition of complex nitrogen sources such as corn steep powder or yeast extract; addition of specific amino acids including proline, glycine, isoleucine, and/or histidine; addition of a complex material containing one or more of these amino acids; addition of other nutrients or other compounds such as phytate, proteases enzymes, or polysaccharase enzymes. In one embodiment, fermentation conditions can include supplementation of a medium with an organic nitrogen source. In another embodiment, fermentation conditions can include supplementation of a medium with an inorganic nitrogen source. In some embodiments, the addition of one material can provide supplements that fit into more than one category, such as providing amino acids and phytate.

In some embodiments, the Q microbe organism can be used to hydrolyze various higher saccharides (higher molecular weight) present in biomass to lower saccharides (lower molecular weight), such as in preparation for fermentation to produce ethanol, hydrogen, or other chemicals such as organic acids including formic acid, acetic acid, and lactic acid. Another advantage of the Q microbe is its ability to hydrolyze polysaccharides and higher saccharides that contain hexose sugar units or that contain pentose sugar units, and that contain both, into lower saccharides and in some cases monosaccharides. These enzymes and/or the hydrolysate can be used in fermentations to produce various products including fuels, and other chemicals. Another advantage of the Q microbe is its ability to produce ethanol, hydrogen, and other fuels or compounds such as organic acids including acetic acid, formic acid, and lactic acid from lower sugars (lower molecular weight) such as monosaccharides. Another advantage of the Q microbe is its ability to perform the combined steps of hydrolyzing a higher molecular weight biomass containing sugars and/or higher saccharides or polysaccharides to lower sugars and fermenting these lower sugars into desirable products including ethanol, hydrogen, and other compounds such as organic acids including formic acid, acetic acid, and lactic acid.

Another advantage of the Q microbe is its ability to grow under conditions that include elevated ethanol concentration, high sugar concentration, low sugar concentration, utilize insoluble carbon sources, and/or operate under anaerobic conditions. These characteristics, in various combinations, can be used to achieve operation with long fermentation cycles and can be used in combination with batch fermentations, fed batch fermentations, self-seeding/partial harvest fermentations, and recycle of cells from the final fermentation as inoculum.

Generally, techniques such as cell recycle and partial harvest fermentation are not frequently used in production scale operations due to various problems inherent with these techniques. For example, “culture exhaustion,” where the cells simply do not provide subsequent fermentations with adequate or similar yields and/or productivity as the original or earlier fermentation is not unusual. In addition, operation with the single culture for extended times, especially when broth is being harvested and there is a risk of breaking sterility, can lead to significant problems with contamination of the culture and fermentations that it is used for. As a result, the suitability of an organism for cell recycle and/or partial harvest fermentation is not generally expected.

In some instances, a process for converting biomass material into ethanol includes pretreating the biomass material (e.g., “feedstock”), hydrolyzing the pretreated biomass to convert polysaccharides to oligosaccharides, further hydrolyzing the oligosaccharides to monosaccharides, and converting the monosaccharides to ethanol. In some instances, the biomass can be hydrolyzed directly to monosaccharides or other saccharides that can be utilized by the fermentation organism to produce ethanol or other products. If a different final product is desired, such as hydrocarbons, hydrogen, methane, hydroxy compounds such as alcohols (e.g. butanol, propanol, methanol, etc.), carbonyl compounds such as aldehydes and ketones (e.g. acetone, formaldehyde, 1-propanal, etc.), organic acids, derivatives of organic acids such as esters (e.g. wax esters, glycerides, etc.) and other functional compounds including, but not limited to, 1,2-propanediol, 1,3-propanediol, lactic acid, formic acid, acetic acid, succinic acid, pyruvic acid, enzymes such as cellulases, polysaccharases, lipases, proteases, ligninases, and hemicellulases, the monosaccharides can be used in the biosynthesis of that particular compound. Biomass material that can be utilized includes woody plant matter, non-woody plant matter, cellulosic material, lignocellulosic material, hemicellulosic material, carbohydrates, pectin, starch, inulin, fructans, glucans, corn, sugar cane, grasses, switchgrass, bamboo, and material derived from these. The final product can then be separated and/or purified, as indicated by the properties for the desired final product. In some instances, compounds related to sugars such as sugar alcohols or sugar acids can be utilized as well.

In this embodiment, more than one of these steps can occur at any given time. For example, hydrolysis of the pretreated feedstock and hydrolysis of the oligosaccharides can occur simultaneously, and one or more of these can occur simultaneously to the conversion of monosaccharides to ethanol.

In some instances, an enzyme can directly convert the polysaccharide to monosaccharides. In some instances, an enzyme can hydrolyze the polysaccharide to oligosaccharides and the enzyme or another enzyme can hydrolyze the oligosaccharides to monosaccharides.

In one embodiment, the enzymes present in the fermentation can be produced separately and then added to the fermentation or they can be produced by microorganisms present in the fermentation. In other embodiments, the microorganisms present in the fermentation can produce some enzymes and some enzymes can be produced separately and added to the fermentation.

For the overall conversion of pretreated biomass to final product to occur at high rates, it is necessary for each of the necessary enzymes for each conversion step to be present with sufficiently high activity. If one of these enzymes is missing or is present in insufficient quantities, the production rate of ethanol, or other desired product will be reduced. The production rate can also be reduced if the microorganisms responsible for the conversion of monosaccharides to product only slowly take up monosaccharides and/or have only limited capability for translocation of the monosaccharides and intermediates produced during the conversion to ethanol.

In one embodiment, the enzymes of the method are produced by the Q microbe itself, including a range of hydrolytic enzymes suitable for the biomass materials used in the fermentation methods. In one embodiment, the Q microbe is grown under conditions appropriate to induce and/or promote production of the enzymes needed for the saccharification of the polysaccharide present. The production of these enzymes can occur in a separate vessel, such as a seed fermentation vessel or other fermentation vessel, or in the production fermentation vessel where ethanol production occurs. When the enzymes are produced in a separate vessel, they can, for example, be transferred to the production fermentation vessel along with the cells, or as a relatively cell free solution liquid containing the intercellular medium with the enzymes. When the enzymes are produced in a separate vessel, they can also be dried and/or purified prior to adding them to the production fermentation vessel. The conditions appropriate for production of the enzymes are frequently managed by growing the cells in a medium that includes the biomass that the cells will be expected to hydrolyze in subsequent fermentation steps. Additional medium components, such as salt supplements, growth factors, and cofactors including, but not limited to phytate, amino acids, and peptides can also assist in the production of the enzymes utilized by the microorganism in the production of the desired products.

### Feedstock and Pretreatment of Feedstock

The feedstock that can contain cellulosic, hemicellulosic, and/or lignocellulosic material can be derived from agricultural crops, crop residues, trees, woodchips, sawdust, paper, cardboard, grasses, and other sources.

Cellulose is a linear polymer of glucose where the glucose units are connected via β(1→4) linkages. Hemicellulose is a branched polymer of a number of sugar monomers including glucose, xylose, mannose, galactose, rhamnose and arabinose, and can have sugar acids such as mannuronic acid and galacturonic acid present as well. Lignin is a cross-linked, racemic macromolecule of mostly p-coumaryl alcohol, conferyl alcohol and sinapyl alcohol. These three polymers occur together in lignocellusic materials in plant biomass. The different characteristics of the three polymers can make hydrolysis of the combination difficult as each polymer tends to shield the others from enzymatic attack.

In one aspect of the invention, methods are provided for the pretreatment of feedstock used in the fermentation and production of the biofuels and ethanol. The pretreatment steps can include mechanical, thermal, pressure, chemical, thermochemical, and/or biochemical tests pretreatment prior to being used in a bioprocess for the production of fuels and chemicals, but untreated biomass material can be used in the process as well. Mechanical processes can reduce the particle size of the biomass material so that it can be more conveniently handled in the bioprocess and can increase the surface area of the feedstock to facilitate contact with chemicals/biochemicals/biocatalysts. Mechanical processes can also separate one type of biomass material from another. The biomass material can also be subjected to thermal and/or chemical pretreatments to render plant polymers more accessible. Multiple steps of treatment can also be used.

Mechanical processes include, are not limited to, washing, soaking, milling, size reduction, screening, shearing, size classification and density classification processes. Chemical processes include, but are not limited to, bleaching, oxidation, reduction, acid treatment, base treatment, sulfite treatment, acid sulfite treatment, basic sulfite treatment, ammonia treatment, and hydrolysis. Thermal processes include, but are not limited to, sterilization, ammonia fiber expansion or explosion (“AFEX”), steam explosion, holding at elevated temperatures, pressurized or unpressurized, in the presence or absence of water, and freezing. Biochemical processes include, but are not limited to, treatment with enzymes and treatment with microorganisms. Various enzymes that can be utilized include cellulase, amylase, β-glucosidase, xylanase, gluconase, and other polysaccharases; lysozyme; laccase, and other lignin-modifying enzymes; lipoxygenase, peroxidase, and other oxidative enzymes; proteases; and lipases. One or more of the mechanical, chemical, thermal, thermochemical, and biochemical processes can be combined or used separately. Such combined processes can also include those used in the production of paper, cellulose products, microcrystalline cellulose, and cellulosics and can include pulping, kraft pulping, acidic sulfite processing. The feedstock can be a side stream or waste stream from a facility that utilizes one or more of these processes on a biomass material, such as cellulosic, hemicellulosic or lignocellulosic material. Examples include paper plants, cellulosics plants cotton processing plants, and microcrystalline cellulose plants. The feedstock can also include cellulose-containing or cellulosic containing waste materials. The feedstock can also be biomass materials, such as wood, grasses, corn, starch, or sugar, produced or harvested as an intended feedstock for production of ethanol or other products such as by Clostridium phytofermentans.

In additional embodiments, methods of the invention can utilize pretreatment processes disclosed in U.S. patents and patent applications US20040152881, US20040171136, US20040168960, US20080121359, US20060069244, US20060188980, US20080176301, U.S. Pat. Nos. 5,693,296, 6,262,313, US20060024801, U.S. Pat. Nos. 5,969,189, 6,043,392, US20020038058, U.S. Pat. No. 5,865,898, U.S. Pat. No. 5,865,898, U.S. Pat. Nos. 6,478,965, 5,986,133, US20080280338, each of which is incorporated by reference herein in its entirety

In another embodiment, the AFEX process can be used for pretreatment of biomass. In a preferred embodiment, the AFEX process is used in the preparation of cellulosic, hemicellulosic or lignocellulosic materials for fermentation to ethanol or other products. The process generally includes combining the feedstock with ammonia, heating under pressure, and suddenly releasing the pressure. Water can be present in various amounts. The AFEX process has been the subject of numerous patents and publications.

In another embodiment, the pretreatment of biomass comprises the addition of calcium hydroxide to a biomass to render the biomass susceptible to degradation. Pretreatment comprises the addition of calcium hydroxide and water to the biomass to form a mixture, and maintaining the mixture at a relatively high temperature. Alternatively, an oxidizing agent, selected from the group consisting of oxygen and oxygen-containing gasses, can be added under pressure to the mixture. Examples of carbon hydroxide treatments are disclosed in U.S. Pat. No. 5,865,898 to Holtzapple and S. Kim and M. T. Holzapple, Bioresource Technology, 96, (2005) 1994, incorporated by reference herein in its entirety.

In other embodiments, pretreatment of biomass comprises dilute acid hydrolysis. Example of dilute acid hydrolysis treatment are disclosed in T. A. Lloyd and C. E Wyman, Bioresource Technology, (2005) 96, 1967), incorporated by reference herein in its entirety.

In other embodiments, pretreatment of biomass comprises pH controlled liquid hot water treatment. Examples of pH controlled liquid hot water treatments are disclosed in N. Mosier et al., Bioresource Technology, (2005) 96, 1986, incorporated by reference herein in its entirety.

In other embodiments, pretreatment of biomass comprises aqueous ammonia recycle process (ARP). Examples of aqueous ammonia recycle process are described in T. H. Kim and Y. Y. Lee, Bioresource Technology, (2005)96, 2007, incorporated by reference herein in its entirety.

In some embodiments, the above mentioned methods have two steps: a pretreatment step that leads to a wash stream, and an enzymatic hydrolysis step of pretreated-biomass that produces a hydrolysate stream. In the above methods, the pH at which the pretreatment step is carried out includes acid hydrolysis, hot water pretreatment, or alkaline reagent based methods (AFEX, ARP, and lime pretreatments). Dilute acid and hot water treatment methods solubilize mostly hemicellulose, whereas methods employing alkaline reagents remove most lignin during the pretreatment step. As a result, the wash stream from the pretreatment step in the former methods contains mostly hemicellulose-based sugars, whereas this stream has mostly lignin for the high-pH methods. The subsequent enzymatic hydrolysis of the residual biomass leads to mixed sugars (C5 and C6) in the alkali based pretreatment methods, while glucose is the major product in the hydrolyzate from the low and neutral pH methods. The enzymatic digestibility of the residual biomass is somewhat better for the high-pH methods due to the removal of lignin that can interfere with the accessibility of cellulase enzyme to cellulose.

In some embodiments, pretreatment of biomass comprises ionic liquid pretreatment. Biomass can be pretreated by incubation with an ionic liquid, followed by IL extraction with a wash solvent such as alcohol or water. The treated biomass can then be separated from the ionic liquid/wash-solvent solution by centrifugation or filtration, and sent to the saccharification reactor or vessel. Examples of ionic liquid pretreatment are disclosed in US publication No. 2008/0227162, incorporated herein by reference in its entirety.

Examples of pretreatment methods are disclosed in U.S. Pat. No. 4,600,590 to Dale, U.S. Pat. No. 4,644,060 to Chou, U.S. Pat. No. 5,037,663 to Dale. U.S. Pat. No. 5,171,592 to Holtzapple, et al., et al., U.S. Pat. No. 5,939,544 to Karstens, et al., U.S. Pat. No. 5,473,061 to Bredereck, et al., U.S. Pat. No. 6,416,621 to Karstens., U.S. Pat. No. 6,106,888 to Dale, et al., U.S. Pat. No. 6,176,176 to Dale, et al., PCT publication WO2008/020901 to Dale, et al., Felix, A., et al., Anim. Prod. 51, 47-61 (1990)., Wais, A. C., Jr., et al., Journal of Animal Science, 35, No. 1, 109-112 (1972), which are incorporated herein by reference in their entireties.

In some embodiments, pretreatment of biomass comprises enzyme hydrolysis. In one embodiment a biomass can be pretreated with an enzyme or a mixture of enzymes, e.g., endonucleases, exonucleases, cellobiohydrolases, cellulase, beta-glucosidases, glycoside hydrolases, glycosyltransferases, lyases, esterases and proteins containing carbohydrate-binding modules. In some embodiments, the enzyme or mixture of enzymes can be individual enzymes with distinct activities. In some embodiments, the enzyme or mixture of enzymes can be enzyme domains with a particular catalytic activity. For example, an enzyme with multiple activities can have multiple enzyme domains, including for example glycoside hydrolases, glycosyltransferases, lyases and/or esterases catalytic domains.

In some embodiments, pretreatment of biomass comprises enzyme hydrolysis with one or more enzymes from C. phytofermentans. In some embodiments, pretreatment of biomass comprises enzyme hydrolysis with one or more enzymes from C. phytofermentans, wherein the one or more enzyme is selected from the group consisting of endonucleases, exonucleases, cellobiohydrolases, beta-glucosidases, glycoside hydrolases, glycosyltransferases, lyases, esterases and proteins containing carbohydrate-binding modules. In some embodiments, biomass can be pretreated with a hydrolase identified in C. phytofermentans. Examples of hydrolases identified in C. phytofermentans include but are not limited to Cphy3367, Cphy3368, Cphy0430, Cphy3854, Cphy0857, Cphy0694, and Cphy1929 (www.genome.jp/).

In some embodiments, pretreatment of biomass comprises enzyme hydrolysis with one or more of enzymes listed in Table 1, Table 2, Table 3, or Table 4. Tables 1-4 show examples of known activities of some of the glycoside hydrolases, lyases, esterases, and proteins containing carbohydrate-binding modules family members predicted to be present in C. phytofermentans, respectively. Known activities are listed by activity and corresponding PC number as determined by the International Union of Biochemistry and Molecular Biology.

TABLE 1
Known activities of glycoside hydrolase family members

| | | Number of |
|---|---|---|
| Glycoside | | domains |
| Hydrolase | | predicted in |
| Family | Known activities | C. phytofermentans |
| 1 | beta-glucosidase (EC 3.2.1.21); beta-galactosidase (EC 3.2.1.23); beta- | 1 |
| | mannosidase (EC 3.2.1.25); beta-glucuronidase (EC 3.2.1.31); beta-D- | |
| | fucosidase (EC 3.2.1.38); phlorizin hydrolase (EC 3.2.1.62); 6-phospho-- | |
| | galactosidase (EC 3.2.1.85); 6-phospho-beta-glucosidase (EC 3.2.1.86); | |
| | strictosidinebeta-glucosidase (EC 3.2.1.105); lactase (EC 3.2.1.108); | |
| | amygdalinbeta-glucosidase (EC 1 3.2.1.117); prunasin beta-glucosidase | |
| | (EC 3.2.1.118); raucaifricine beta-glucosidase (EC 3.2.1.125); | |
| | thioglucosidase (EC 3.2.1.147); beta-primeverosidase (EC 3.2.1.149); | |
| | isoflavonod 7-0-beta-apiosyl--glucosidase (EC 3.2.1.161); | |
| | hydroxyisourate hydrolase (EC_3.—.—.—); _beta-glycosidase_(EC_3.2.1.—) | |
| 2 | beta-galactosidase (EC 3.2.1.23); beta-mannosidase (EC 3.2.1.25); beta- | 5 |
| | glucuronidase (EC 3.2.1.31); mannosylglycoprotein 5 endo-beta- | |
| | mannosidase (EC 3.2.1.152); exo-beta glucosaminidase_(E 3.2.1.—) | |
| 3 | beta-glucosidase (EC 3.2.1.21); xylan 1,4-beta-xylosidase (EC 3.2.1.37); | 8 |
| | beta-N-acetylhexosaminidase (EC 3.2.1.52); glucan 1,3-beta- | |
| | glucosiclase (EC 3.2.1.58); glucan 1,4-beta-glucosidase (EC 3.2.1.74); | |
| | exo-1,3-1,4-glucanase (EC 3.2.1.—); alpha-L arabinofuranosidase (EC | |
| | 3.2.1.55). | |
| 4 | maltose-6-phosphate glucosidase (EC 3.2.1.122); alpha glucosidase (EC | 3 |
| | 3.2.1.20); alpha-galactosidase (EC 3.2.1.22); 6-phospho-beta-glucosidase | |
| | (EC 3.2.1.86); alpha-glucuronidase (EC 3.2.1.139). | |
| 5 | chitosanase (EC 3.2.1.132); beta-mannosidase (EC 3.2.1.25); Cellulase | 3 |
| | (EC 3.2.1.4); glucan 1,3-beta-glucosidase (EC 3.2.1.58); licheninase (EC | |
| | 3.2.1.73); glucan endo-1,6-beta-glucosidase (EC 3.2.1.75); mannan endo- | |
| | 1,4-beta-mannosidase (EC 3.2.1.78); 3 Endo-1,4-beta-xylanase (EC | |
| | 3.2.1.8); cellulose 1,4-beta-cellobiosidase (EC 3.2.1.91); endo-1,6-beta- | |
| | galactanase (EC 3.2.1.—); beta-1,3-mannanase (EC 3.2.1.—); xyloglucan- | |
| | specific endo-beta-1,4-glucanase (EC 3.2.1.151) | |
| 8 | chitosanase (EC 3.2.1.132); cellulase (EC 3.2.1.4); licheninase (EC | 1 |
| | 3.2.1.73); endo-1,4-beta-xylanase (EC 3.2.1.8); reducing-end-xylose | |
| | releasing exo-oligoxylanase (EC 3.2.1.156) | |
| 9 | endoglucanase (EC 3.2.1.4); cellobiohydrolase (EC 3.2.1.91); beta- | 1 |
| | glucosidase (EC 3.2.1.21) | |
| 10 | xylanase (EC 3.2.1.8); endo-1,3-beta-xylanase (EC 3.2.1.32) | 6 |
| 11 | xylanase (EC 3.2.1.8). | 1 |
| 12 | endoglucanase (EC 3.2.1.4); xyloglucan hydrolase (EC 3.2.1.151); beta- | 1 |
| | 1,3-1,4-glucanase (EC 3.2.1.73); xyloglucan endotransglycosylase (EC | |
| | 2.4.1.207) | |
| 13 | apha-amylase (EC 3.2.1.1); pullulanase (EC 3.2.1.41); cyclomaltodextrin | 7 |
| | glucanotransferase (EC 2.4.1.19); cyclornaltodextrinase (EC 3.2.1.54); | |
| | trehalose-6-phosphate hydrolase (EC 3.2.1.93); oligo-alpha-glucosiclase | |
| | (EC 3.2.1.10); maltogenic amylase (EC 3.2.1.133); neopullulanase (EC | |
| | 3.2.1.135); alpha-glucosidase (EC 3.2.1.20); maltotetraose-forming 3 | |
| | alpha-amylase (EC 3.2.1.60); isoamylase (EC 3.2.1.68); glucodextranase | |
| | (EC 12.170); maltohexaose-forming alphaamylase (EC 3.2.1.98); | |
| | branching enzyme (EC 2.4.1.18); trehalose synthase (EC 5.4.99.16); 4-- | |
| | glucanotransferase (EC 2.4.1.25); maltopentaose-forming-amylase (EC | |
| | 3.2.1.—); amylosucrase (EC 2.4.1.4): sucrose phosphorylase (EC 2.4.1.7); | |
| | malto-oligosyltrehalose trehalohydrolase (EC 3.2.1.141); isomaltulose | |
| | synthase (EC 5.4.99.11). | |
| 16 | xyloglucan:xyloglucosyltransferase (EC 2.4.1.207); keratan-sulfate endo- | 1 |
| | 1,4-beta-galactosidase (EC 3.2.1.103); Glucan endo-1,3-beta-D- | |
| | glucosidase (EC 3.2.1.39); endo-1,3(4)-beta-glucanase (EC 3.21.6); | |
| | Licheninase (EC 3.2.1.73): agarase (EC 3.2.1.81); betacarrageenase (EC | |
| | 3.2.1.83); xyioglucanase (EC 3.2.1.151) | |
| 18 | chitinase (EC 3.2.1.14); endo-beta-N-acetylglucosaminidase (EC | 6 |
| | 3.2.1.96); non-catalytic proteins: xylanase inhibitors; concanavalin B; | |
| | narbonin | |
| 19 | chitinase(EC 3.2.1.14). | 2 |
| 20 | beta-hexosaminidase (EC 3.2.1.52); lacto-N-biosidase (EC 3.2.1.140); - | 3 |
| | 1,6-N-acetylglucosaminidase) (EC 3.2.1.—) | |
| 25 | lysozyme (EC 3.2.1.17) | 1 |
| 26 | beta-mannanase (EC 3.2.1.78); beta-1,3-xylanase (EC 3.2.1.32) | 3 |
| 28 | polygalacturonase (EC 3.2.1.15); exo-polygalacturonase (EC 3.2.1.67); | 5 |
| | exo-polygalacturonosidase (EC 3.2.1.82); rhamnogalacturonase (EC | |
| | 3.2.1.—); endo-xylogalacturonan hydrolase (EC 3.2.1.—); | |
| | rhamnogalacturonan alpha-L-rhamnopyranohydrolase (EC 3.2.1.40) | |
| 29 | alpha-L-fucosidase (EC 3.2.1.51) | 3 |
| 30 | glucosylceramidase (EC 3.2.1.45); beta-1,6-glucanase (EC 3.2.1.75); | 2 |
| | beta-xylosidase (EC 3.2.1.37) | |
| 31 | alpha-glucosidase (EC 3.2.1.20): alpha-1,3-glucosidase (EC 3.2.1.84); | 3 |
| | sucrase-isomaltase (EC 3.2.1.48) (EC 3.2.1.10); alpha-xylosidase (EC | |
| | 3.2.1.—); alpha-glucan lyase (EC 4.2.2.13); isomaltosyltransferase_(EC | |
| | 2.4.1.—). | |
| 36 | alpha-galactosidase (EC 3.2.1.22); alpha-N-acetylgalactosaminidase (EC | 2 |
| | 3.2.1.49); stachyose synthase (EC 2.4.1.67); raffinose synthase (EC | |
| | 2.4.1.82) | |
| 38 | alpha-mannosidase (EC 3.2.1.24); alpha-mannosidase (EC 3.2.1.114) | 1 |
| 43 | beta-xylosidase (EC 3.2.1.37); beta-1,3-xylosidase (EC 3.2.1.—); alpha-L- | 8 |
| | arabinofuranosidase (EC 3.2.1.55); arabinanase (EC 3.2.1.99); xylanase | |
| | (EC 3.2.1.8); galactan 1,3-beta-galactosidase (EC 3.2.1.145) | |
| 48 | endoglucanase (EC 3.2.1.4); chitinase (EC 3.2.1.14); cellobiohydrolases | 1 |
| | some cellobiohydrolases of this family have been reported to act from the | |
| | reducing ends of cellulose (EC 3.2.1.—), while others have been reported to | |
| | operate from the non-reducing ends to liberate cellobiose or cellotriose or | |
| | cellotetraose (EC 3.2.1.—). This family also contains endo-processive | |
| | celtulases (EC 3.2.1.—), whose activity is hard to distinguish from that of | |
| | cellobiohydrolases. | |
| 51 | alpha-L-arabinofuranosidase (EC 3.2.1.55); endoglucanase (EC 3.2.1.4) | 1 |
| 65 | trehalase (EC 3.2.1.28); maltose phosphorylase (EC 2.4.1.8); trehalose | 4 |
| | phosphorylase (EC 2.4.1.64); kojibiose phosphorylase (EC 2.4.1.230) | |
| 67 | alpha-glucuronidase (EC 3.2.1.139); xylan alpha-I,2-glucuronosidase | 1 |
| | (EC_3.2.1.131) | |
| 73 | peptidoglycan hydrolases with endo-beta-N-acetylglucosam inidase (EC | 1 |
| | 3.2.1.—) specificity; there is only one, unconfirmed, report of beta-i,4-N- | |
| | acetylmuramoylhydrolase (EC 3.2.1.17) activity | |
| 77 | amylomaltase or 4-aipha-glucanotransferase (EC 2.4.1.25) | 1 |
| 85 | endo-beta-N-acetylglucosaminidase (EC 3.2.1.96) | 1 |
| 87 | mycodextranase (EC 3.2.1.61); alpha-1,3-glucanase (EC 3.2.1.59) | 3 |
| 88 | d-4,5 unsaturated beta-glucuronyl hydrolase (EC 3.2.1.—) | 4 |
| 94 | cellobiose phosphorylase (EC 2.4.1.20); cellodextrin phosphorylase (EC | 5 |
| | 2.4.1.49); chitobiose phosphorylase (EC 2.4.1.—); cyclic beta-1,2-glucan | |
| | synthase (EC 2.4.1.—) | |
| 95 | alpha-1,2-L-fucosidase (EC 3.2.1.63); alpha-L-fucosidase (EC 3.2.1.51) | 2 |
| 105 | unsaturated rhamnogalacturonyl hydrolase (EC 3.2.1.—) | 3 |
| 106 | alpha-L-rhamnosidase (EC 3.2.1.40) | 1 |
| 112 | lacto-N-biose phosphorylase or galacto-N-biose phosphorylase (EC | 3 |
| | 2.4.1.211) | |

TABLE 2
Known activities of polysaccharide lyase family members
Polysaccharide lyase family Known activities Number of
domains predicted in C. phytofermentans

| | | Number of |
|---|---|---|
| | | domains |
| Polysaccharide | | predicted in |
| lyase family | Known activities | C. phytofermentans |
| 1 | pectate lyase (EC 4.2.2.2); exo-pectate lyase (EC 4.2.2.9); pectin lyase | 1 |
| | (EC_4.2.2.10). | |
| 7 | alginate lyase (EC 4.2.2.3); -L-guluronate lyase (EC 4.2.2.11) | 1 |
| 9 | pectate lyase (EC 4.2.2.2); exopolygalacturonate lyase (EC_4.2.2.9). | 4 |
| 11 | pectate lyase (EC 4.2.2.2); exopolygalacturonate lyase (EC_4.2.2.9). | 1 |
| 12 | Heparin-sulfate lyase (EC 4.2.2.8) | 1 |
| 15 | oligo-alginate lyase (EC 4.2.2.—) | 1 |
| 17 | alginate lyase (EC 4.2.2.3). | 1 |

TABLE 3
Known activities of carbohydrate esterase family members

| | | Number of |
|---|---|---|
| Carbohydrate | | domains |
| esterase | | predicted in |
| family | Known activities | C. phytofermentans |
| 2 | acetyl xylan esterase (EC 3.1.1.72). | 2 |
| 4 | acetyl xylan esterase (EC 3.1.1.72); chitin deacetylase (EC 3.5.1.41); | 8 |
| | chitooligosaccharide deacetylase (EC 3.5.1.—); peptidoglycan GIcNAc | |
| | deacetylase (EC 3.5.1.—); peptidoglycan N-acetylmuramic acid deacetylase | |
| | (EC 3.5.1.—). | |
| 8 | pectin methylesterase (EC 3.1.1.11). | 1 |
| 9 | N-acetylglucosamine 6-phosphate deacetylase (EC 3.5.1.25); N- | 2 |
| | acetylgalactosamine-6-phosphate deacetylase (EC 3.5.1.80). | |
| 12 | pectin acetylesterase (EC 3.1.1.—); rhamnogalacturonan acetylesterase (EC | 1 |
| | 3.1.1.—); acetyl xylan esterase (EC 3.1.1.72) | |
| 15 | 4-0-methyl-glucuronyl esterase(3.1.1.—) | 1 |

TABLE 4
Known activities of carbohydrate-binding module family members

| | | Number of |
|---|---|---|
| | | domains |
| | | predicted in |
| CBM family | Known activities | C. phytofermentans |
| 2 | Modules of approx. 100 residues found in many bacterial enzymes with | 1 |
| | putative cellulose, chum and/or xylan binding activities. | |
| 3 | Modules of approx. 150 residues found in bacterial enzymes. The cellulose- | 5 |
| | binding function has been demonstrated in many cases. In one instance | |
| | binding to chitin has been reported. | |
| 4 | Modules of approx. 150 residues found in bacterial enzymes. Binding of these | 4 |
| | modules has been demonstrated with xylan, -1,3-glucan, -1,3-1,4-glucan, -1,6- | |
| | glucan and amorphous cellulose but not with crystalline cellulose. | |
| 5 | Modules of approx. 60 residues found in bacterial enzymes. Distantly related | 1 |
| | to the CBM12 family. | |
| 6 | Modules of approx. 120 residues. The cellulose-binding function has been | 1 |
| | demonstrated in one case on amorphous cellulose and xylan. Some of these | |
| | modules also bind -1,3-glucan. | |
| 12 | Modules of approx. 40-60 residues. The majority of these modules is found | 2 |
| | among chitinases where the function is chitin-binding. Distantly related to the | |
| | CBM5 family. | |
| 13 | Modules of approx. 150 residues which often appear as a threefold internal | 1 |
| | repeat, an exception includes, xylanase II of Act inomadura sp. FC7 (GenBank | |
| | U08894). These modules were first identified in several plant lectins such as | |
| | ricin or agglutinin of Ricinus communis which bind galactose residues. The | |
| | three-dimensional structure of a plant lectin has been determined and displays | |
| | a pseudo-threefold symmetry in accord with the observed sequence threefold | |
| | repeat. These modules have since been found in a number of other proteins of | |
| | various functions including glycoside hydrolases and glycosyltransferases. | |
| | While in the plant lectins this module binds mannose, binding to xylan has | |
| | been demonstrated in the Streptomyces lividans xylanase A and | |
| | arabinofuranosidase B. Binding to GalNAc has been shown for the | |
| | corresponding module of GalNAc transferase 4. For the other proteins, the | |
| | binding specificity of these modules has not been established. The pseudo | |
| | three-fold symmetry of the CBM13 module has now been confirmed in the 3- | |
| | D structure of the intact, two-domain, xylanase of Streptomyces | |
| | olivaceoviridis. | |
| 22 | A xylan binding function has been demonstrated in several cases and affinity | 1 |
| | with mixed -1,3/-1,4-glucans in one. In several cases a thermostabilizing | |
| | effect has also been seen. | |
| 32 | Binding to galactose and lactose has been demonstrated for the module of | 5 |
| | Micromonospora viridifaciens sialidase (PM ID: 16239725); binding to | |
| | polygalacturonic acid has been shown for a Yersinia member (PMID: | |
| | 17292916); binding to LacNAc (-D-galactosyl-1,4--D-N-acetylglucosamine) | |
| | has been shown for an N-acetylglucosaminidase from Clostridium perfingens | |
| | (PM ID: 16990278). | |
| 35 | Modules of approx. 130 residues. A module that is conserved in three | 4 |
| | Cellvibrio xylan-degrading enzymes binds to xylan and the interaction is | |
| | calcium dependent, while a module from a Cellvibrio mannanase binds to | |
| | decorated soluble mannans and mannooligosaccharides. A module in a | |
| | Phanerochaete chrysosporium galactan 1,3--galactosidase binds to -galactan. | |
| 36 | Modules of approx. 130 residues. A module that is conserved in three | 1 |
| | Cellvibrio xylan-degraciing enzymes binds to xylan and the interaction is | |
| | calcium dependent, while a module from a Cellvibrio mannanase binds to | |
| | decorated soluble mannans and mannooligosaccharides. A module in a | |
| | Phanerochaete chrysosporium galactan 1,3--galactosidase binds to -galactan. | |
| 41 | Modules of approx. 100 residues found in primarily in bacterial pullulanases. | 11 |
| | The N-terminal module from Thermotoga maritima Pul13 has been shown to | |
| | bind to the -glucans amylose, amylopectin, pullulan, and oligosaccharide | |
| | fragments derived from these polysaccharides. | |
| 46 | Modules of approx. 100 residues, found at the C-terminus of several GH5 | 1 |
| | cellulases. Cellulose-binding function demonstrated in one case. | |
| 48 | Modules of approx. 100 residues with glycogen-binding function, appended to | 2 |
| | GH13 modules. Also found in the beta subunit (glycogen-binding) of AMP- | |
| | activated protein kinases (AMPK) | |
| 50 | Modules of approx. 50 residues found attached to various enzymes from | 4 |
| | families GH18, GH19, GH23, GH24, GH25 and GH73, i.e. enzymes cleaving | |
| | either chitin or peptidoglycan. Binding to chitopentaose demonstrated in the | |
| | case of Pteris ryukyuensis chitinase A [Ohnuma T et al.; PMID: 18083709]. | |
| | CBM5O modules are also found in a multitude of other enzymes targeting the | |
| | petidoglycan such as peptidases and amidases. | |

In some embodiments, enzymes that degrade polysaccharides are used for the pretreatment of biomass and can include enzymes that degrade cellulose, namely, cellulases. Examples of some cellulases include endocellulases (EC 3.2.1.4) and exo-cellulases (EC 3.2.1.91), and hydrolyze beta-1,4-glucosidic bonds.

Examples of predicted endo-cellulases in C. phytofermentans that can be used in the pretreatment of biomass include genes within the GH5 family, such as, Cphy3368; Cphy1163, and Cphy2058; the GH8 family, such as Cphy3207; and the GH9 family, such as Cphy3367. Examples of exocellulases in C. phytofermentans that can be used in the pretreatment of biomass include genes within the GH48 family, such as Cphy3368. Some exo-cellulases hydrolyze polysaccharides to produce 2 to 4 units oligosaccharides of glucose, resulting in cellodextrins disaccharides (cellobiose), trisaccharides (cellotriose), or tetrasaccharides (cellotetraose). Members of the GH5, GH9 and GH48 families can have both exo- and endo-cellulase activity.

In some embodiments, enzymes that degrade polysaccharides are use for the pretreatment of biomass and can include enzymes that have the ability to degrade hemicellulose, namely, hemicellulases (Leschine, S. B. in Handbook on Clostridia (ed Dune, P.) (CRC Press, Boca Raton, 2005)). Hemicellulose can be a major component of plant biomass and can contain a mixture of pentoses and hexoses, for example, D-xylopyranose, L-arabinofuranose, D-mannopyranose, Dglucopyranose, D-galactopyranose, D-glucopyranosyluronic acid and other sugars (Aspinall, G. O. The Biochemistry of Plants 473, 1980; Han, J. S. & Rowell, J. S. in Paper and composites from agro-based resources 83, 1997). In certain embodiments, predicted hemicellulases identified in C. phytofermentans that can be used in the pretreatment of biomass include enzymes active on the linear backbone of hemicellulose, for example, endo-beta-1,4-D-xylanase (EC 3.2.1.8), such as GH5, GH10, GH11, and GH43 family members; 1,4-beta-D-xyloside xylohydrolase (EC 3.2.1.37), such as GH30, GH43, and GH3 family members; and beta-mannanase (EC 3.2.1.78), such as GH26 family members.

In more embodiments, predicted hemicellulases identified in C. phytofermentans that can be used in the pretreatment of biomass include enzymes active on the side groups and substituents of hemicellulose, for example, alpha-L-arabinofuranosidase (EC 3.2.1.55), such as GH3, GH43, and GH51 family members; alpha-xylosidase, such as GH31 family members; alphafucosidase (EC 3.2.1.51), such as GH95 and GH29 family members; galactosidase, such as GH1, GH2, GH4, GH36, GH43 family members; and acetyl-xylan esterase (EC 3.1.1.72), such as CE2 and CE4.

In some embodiments, enzymes that degrade polysaccharides are used for the pretreatment of biomass and can include enzymes that have the ability to degrade pectin, namely, pectinases. In plant cell walls, the cross-linked cellulose network can be embedded in a matrix of pectins that can be covalently cross-linked to xyloglucans and certain structural proteins. Pectin can comprise homogalacturonan (HG) or rhamnogalacturonan (RH).

In more embodiments, preteatment of biomass comprises pectinases identified in C. phytofermentans which can hydrolyze HG. HG can be composed of D-galacturonic acid (D-galA) units which can be acetylated and methylated. Enzymes that hydrolyze HG can include, for example, 1,4-alpha-D galacturonan lyase (EC 4.2.2.2), such as PL1, PL9, and PL11 family members; glucuronyl hydrolase, such as GH88 and GH105 family members; pectin acetylesterase such as CE12 family members; and pectin methylesterase, such as CE8 family members.

In even more embodiments, pretreatment of biomass comprises pectinases identified in C. phytofermentans which can hydrolyze RH. RH can be a backbone composed of alternating 1,2-alpha-L-rhamnose (L-Rha) and 1,4-alpha-D-galacturonic residues (Lau, J. M., McNeil M., Darvill A. G. & Albersheim P. Structure of the backbone of rhamnogalacturonan I, a pectic polysaccharide in the primary cell walls of plants. Carbohydrate research 137, 111 (1985)). The rhamnose residues of the backbones can have galactan, arabinan or arabinogalactan attached to C4 as side chains. Enzymes that hydrolyze HG can include, for example, endorhamnogalacturonase, such as GH28 family members; and rhamnogalacturonan lyase, such as PL11 family members.

In some embodiments, pretreatment of biomass includes enzymes that can hydrolyze starch. C. phytofermentans can degrade starch and chitin (Warnick, T. A., Methe, B. A. & Leschine, S. B. Clostridium phytofermentans sp. nov., a cellulolytic mesophile from forest soil. Int. J. Syst. Evol. Microbiol. 52, 1155-1160 (2002); Leschine, S. B. in Handbook on Clostridia (ed Dürre, P.) (CRC Press, Boca Raton, 2005); Reguera, G. & Leschine, S. B. Chitin degradation by cellulolytic anaerobes and facultative aerobes from soils and sediments. FEMS Micro biol. Lett. 204, 367-374 (2001)). Enzymes that hydrolyze starch include alpha-amylase, glucoamylase, beta-amylase, exo-alpha-1,4-glucanase, and pullulanase. Examples of predicted enzymes identified in C. phytofermentans involved in starch hydrolysis include GH13 family members.

In more embodiments, pretreatment of biomass comprises hydrolases that can include enzymes that hydrolyze chitin. Examples of enzymes that can hydrolyze chitin include GH18 and GH19 family members. In even more embodiments, hydrolases can include enzymes that hydrolyze lichen, namely, lichenase, for example, GH16 family members, such as Cphy3388.

In some embodiments, pretreatment of biomass comprises hydrolases that are proteins containing carbohydrate-binding modules family members (CBM). Without wishing to be bound to any one theory, CBM domains can function to localize enzyme complexes to particular substrates. Examples of predicted CBM families identified in C. phytofermentans that can bind cellulose include CBM2, CBM3, CBM4, CBM6 and CBM46 family members. Examples of predicted CBM families identified in C. phytofermentans that can bind xylan include CBM2, CBM4, CBM6, CBM13, CBM22, CBM35, and CBM36 family members. In more embodiments, CBM domain family members can function to stabilize an enzyme complex.

In some embodiments, after pretreatment by any of the above methods the feedstock contains cellulose, hemicellulose, soluble oligomers, simple sugars, lignans, volatiles and ash. The parameters of the pretreatment can be changed to vary the concentration of the components of the pretreated feedstock. For example, in some embodiments a pretreatment is chosen so that the concentration of soluble oligomers is high and the concentration of lignans is low after pretreatment. Examples of parameters of the pretreatment include temperature, pressure, time, and pH.

In some embodiments, the parameters of the pretreatment are changed to vary the concentration of the components of the pretreated feedstock such that concentration of the components in the pretreated stock is optimal for fermentation with a microbe such as a Q microbe.

In some embodiments, the parameters of the pretreatment are changed such that concentration of accessible cellulose in the pretreated feedstock is 1%, 5%, 10%, 12%, 13%, 14%, 15%, 16%, 17%, 19%, 20%, 30%, 40% or 50%. In some embodiments, the parameters of the pretreatment are changed such that concentration of accessible cellulose in the pretreated feedstock is 5% to 30%. In some embodiments, the parameters of the pretreatment are changed such that concentration of accessible cellulose in the pretreated feedstock is 10% to 20%.

In some embodiments, the parameters of the pretreatment are changed such that concentration of hemicellulose in the pretreated feedstock is 1%, 5%, 10%, 12%, 13%, 14%, 15%, 16%, 17%, 19%, 20%, 21%, 22%, 23%, 24%, 25%, 26%, 27%, 28%, 29%, 30%, 40% or 50%. In some embodiments, the parameters of the pretreatment are changed such that concentration of hemicellulose in the pretreated feedstock is 5% to 40%. In some embodiments, the parameters of the pretreatment are changed such that concentration of hemicellulose in the pretreated feedstock is 10% to 30%.

In some embodiments, the parameters of the pretreatment are changed such that concentration of soluble oligomers in the pretreated feedstock is 1%, 10%, 15%, 20%, 25%, 30%, 35%, 40%, 45%, 50%, 55%, 60%, 65%, 70%, 75%, 80%, 85%, 90%, 95%, or 99%. Examples of soluble oligomers include, but are not limited to, cellobiose and xylobiose. In some embodiments, the parameters of the pretreatment are changed such that concentration of soluble oligomers in the pretreated feedstock is 30% to 90%. In some embodiments, the parameters of the pretreatment are changed such that concentration of soluble oligomers in the pretreated feedstock is 45% to 80%. In some embodiments, the parameters of the pretreatment are changed such that concentration of soluble oligomers in the pretreated feedstock is 45% to 80% and the soluble oligomers are primarily cellobiose and xylobiose.

In some embodiments, the parameters of the pretreatment are changed such that concentration of simple sugars in the pretreated feedstock is 1%, 5%, 10%, 12%, 13%, 14%, 15%, 16%, 17%, 19%, 20%, 30%, 40% or 50%. In some embodiments, the parameters of the pretreatment are changed such that concentration of simple sugars in the pretreated feedstock is 0% to 20%. In some embodiments, the parameters of the pretreatment are changed such that concentration of simple sugars in the pretreated feedstock is 0% to 5%. Examples of simple sugars include, but are not limited to, C5 and C6 monomers and dimers.

In some embodiments, the parameters of the pretreatment are changed such that concentration of lignans in the pretreated feedstock is 1%, 5%, 10%, 12%, 13%, 14%, 15%, 16%, 17%, 19%, 20%, 30%, 40% or 50%. In some embodiments, the parameters of the pretreatment are changed such that concentration of lignans in the pretreated feedstock is 0% to 20%. In some embodiments, the parameters of the pretreatment are changed such that concentration of lignans in the pretreated feedstock is 0% to 5%. In some embodiments, the parameters of the pretreatment are changed such that concentration of lignans in the pretreated feedstock is less than 1% to 2%. In some embodiments, the parameters of the pretreatment are changed such that the concentration of phenolics is minimized.

In some embodiments, the parameters of the pretreatment are changed such that concentration of furfural and low molecular weight lignans in the pretreated feedstock is less than 10%, 9%, 8%, 7%, 6%, 5%, 4%, 3%, 2%, or 1%. In some embodiments, the parameters of the pretreatment are changed such that concentration of furfural and low molecular weight lignans in the pretreated feedstock is less than 1% to 2%.

In some embodiments, the parameters of the pretreatment are changed such that concentration of accessible cellulose is 10% to 20%, the concentration of hemicellulose is 10% to 30%, the concentration of soluble oligomers is 45% to 80%, the concentration of simple sugars is 0% to 5%, and the concentration of lignans is 0% to 5% and the concentration of furfural and low molecular weight lignans in the pretreated feedstock is less than 1% to 2%.

In some embodiments, the parameters of the pretreatment are changed to obtain a high concentration of hemicellulose and a low concentration of lignans. In some embodiments, the parameters of the pretreatment are changed to obtain a high concentration of hemicellulose and a low concentration of lignans such that concentration of the components in the pretreated stock is optimal for fermentation with a microbe such as a Q microbe.

In some embodiments, a feedstock is pretreated at a pH of 8 to 12 to obtain a high concentration of hemicellulose and a low concentration of lignans in the pretreated feedstock. In some embodiments, a feedstock is pretreated at a pH of 8 to 12 to obtain a high concentration of hemicellulose and a low concentration of lignans such that concentration of the components in the pretreated stock is optimal for fermentation with a microbe such as a Q microbe. Other parameters such as temperature and time can be changed to obtain the desire results. For example, in some embodiments a feedstock is pretreated at a pH of 8 to 12 at a low temperature for a long time to obtain a high concentration of hemicellulose and a low concentration of lignans in the pretreated feedstock.

In some embodiments, the parameters of the pretreatment are changed to obtain the maximum number of C5 constituent carbohydrates. In some embodiments, the parameters of the pretreatment are changed such that the crystallinity of the components in the feedstock is no greater than natural amounts.

In some embodiments, the feedstock is treated with alkaline compounds such as NaOH, KOH, and Ca(OH)₂ under varying conditions to obtain the desire concentration of components in the pretreated feedstock. For example, in some embodiments he feedstock is treated with alkaline compounds such as NaOH, KOH, and Ca(OH)₂ under varying conditions so that the concentration of hemicellulose is high and the concentration of lignans is low after treatment. Alkaline treatments can be performed in combination with agents such as hydrogen peroxide or urea.

In some embodiments, the feedstock is treated with alkaline compounds such as NaOH, KOH, and Ca(OH)₂ under varying such that concentration of the components in the pretreated stock is optimal for fermentation with a microbe such as a Q microbe. Alkaline treatments can be performed in combination with agents such as hydrogen peroxide or urea.

In some embodiments, the feedstock is treated with NaOH such that the concentration of the components in the pretreated stock is optimal for fermentation with Q microbe. The NaOH pretreatment can be performed in combination with agents such as hydrogen peroxide or urea. The NaOH pretreatment, alone or in combination with hydrogen peroxide or urea, can be performed at 60° C., 80° C., 90° C., 100° C., 120° C., 140° C., 160° C. or 180° C. The NaOH pretreatment, alone or in combination with hydrogen peroxide or urea, can be performed for 10, 15, 20, 30, 35, 40, 50 minutes or 1, 5, 7, 9, 10, 11, 15, 20, 25, 30, 35 or 36 hours.

In some embodiments, the feedstock is treated with KOH such that the concentration of the components in the pretreated stock is optimal for fermentation with Q microbe. In one embodiment a KOH pretreatment can be performed in combination with agents such as hydrogen peroxide or urea. In another embodiment a Ca(OH)₂ pretreatment, alone or in combination with hydrogen peroxide or urea, can be performed at about 60° C. to 180° C. In another embodiment a KOH pretreatment, alone or in combination with hydrogen peroxide or urea, can be performed at about 60° C., 80° C., 90° C., 100° C., 120° C., 140° C., 160° C. or 180° C. In one embodiment a KOH pretreatment, alone or in combination with hydrogen peroxide or urea, can be performed for about 1-60 minutes. In another embodiment a KOH pretreatment, alone or in combination with hydrogen peroxide or urea, can be performed for about 1-96 hours. In another embodiment a KOH pretreatment, alone or in combination with hydrogen peroxide or urea, can be performed for about 10, 15, 20, 30, 35, 40, or 50 minutes or about 1, 5, 7, 9, 10, 11, 15, 20, 25, 30, 35, 36, 40, 45, 50, 55, 60, 65, 70, 75, 80, 85, 90, or 96 hours.

In one embodiments, the feedstock is treated with Ca(OH)₂ such that the concentration of the components in the pretreated stock is optimal for fermentation with Q microbe. In another embodiment the Ca(OH)₂ pretreatment can be performed in combination with agents such as hydrogen peroxide or urea. In another embodiment the Ca(OH)₂ pretreatment, alone or in combination with hydrogen peroxide or urea, can be performed at about 60° C. to 180° C. n another embodiment the Ca(OH)₂ pretreatment, alone or in combination with hydrogen peroxide or urea, can be performed at about 60° C., 80° C., 90° C., 100° C., 120° C., 140° C., 160° C. or 180° C. In one embodiment a Ca(OH)₂ pretreatment, alone or in combination with hydrogen peroxide or urea, can be performed for about 1-60 minutes. In another embodiment a Ca(OH)₂ pretreatment, alone or in combination with hydrogen peroxide or urea, can be performed for about 1-96 hours. In another embodiment a Ca(OH)₂ pretreatment, alone or in combination with hydrogen peroxide or urea, can be performed for about 10, 15, 20, 30, 35, 40, or 50 minutes or about 1, 5, 7, 9, 10, 11, 15, 20, 25, 30, 35, 36, 40, 45, 50, 55, 60, 65, 70, 75, 80, 85, 90, or 96 hours.

### Recovery of Ethanol or Other Fermentive End Products

In another aspect of the invention, methods are provided for the recovery of the fermentive end products, such as an alcohol (e.g. ethanol, propanol, methanol, butanol, etc.) another biofuel or chemical product. In one embodiment, broth will be harvested at some point during of the fermentation, and fermentive end product or products will be recovered. The broth with ethanol to be recovered will include both ethanol and impurities. The impurities include materials such as water, cell bodies, cellular debris, excess carbon substrate, excess nitrogen substrate, other remaining nutrients, non-ethanol metabolites, and other medium components or digested medium components. During the course of processing the broth, the broth can be heated and/or reacted with various reagents, resulting in additional impurities in the broth.

In one embodiment, the processing steps to recover ethanol frequently includes several separation steps, including, for example, distillation of a high concentration ethanol material from a less pure ethanol-containing material. In other embodiments, the high concentration ethanol material can be further concentrated to achieve very high concentration ethanol, such as 98% or 99% or 99.5% (wt.) or even higher. Other separation steps, such as filtration, centrifugation, extraction, adsorption, etc. can also be a part of some recovery processes for ethanol as a product or biofuel, or other biofuels or chemical products.

In one embodiment a process can be scaled to produce commercially useful biofuels. In another embodiment the Q microbe is used to produce an alcohol, e.g., ethanol, butanol, propanol, methanol, or a fuel such as hydrocarbons hydrogen, methane, and hydroxy compounds. In another embodiment the Q microbe is used to produce a carbonyl compound such as an aldehyde or ketone (e.g. acetone, formaldehyde, 1-propanal, etc.), an organic acid, a derivative of an organic acid such as an ester (e.g. wax ester, glyceride, etc.), 1,2-propanediol, 1,3-propanediol, lactic acid, formic acid, acetic acid, succinic acid, pyruvic acid, or an enzyme such as a cellulase, polysaccharase, lipases, protease, ligninase, and hemicellulase.

In one embodiment, a fed-batch fermentation for production of fermentive end product is described. In another embodiment, a fed-batch fermentation for production of ethanol is described. Fed-batch culture is a kind of microbial process in which medium components, such as carbon substrate, nitrogen substrate, vitamins, minerals, growth factors, cofactors, etc. or biocatalysts (including, for example, fresh organisms, enzymes prepared by the Q microbe in a separate fermentation, enzymes prepared by other organisms, or a combination of these) are supplied to the fermentor during cultivation, but culture broth is not harvested at the same time and volume. To improve bioconversion from soluble and insoluble substrates, such as those that can be used in biofuels production, various feeding strategies can be utilized to improve yields and/or productivity. This technique can be used to achieve a high cell density within a given time. It can also be used to maintain a good supply of nutrients and substrates for the bioconversion process. It can also be used to achieve higher titer and productivity of desirable products that might otherwise be achieved more slowly or not at all.

In another embodiment, the feeding strategy balances the cell production rate and the rate of hydrolysis of the biomass feedstock with the production of ethanol. Sufficient medium components are added in quantities to achieved sustained cell production and hydrolysis of the biomass feedstock with production of ethanol. In some embodiments, sufficient carbon and nitrogen substrate are added in quantities to achieve sustained production of fresh cells and hydrolytic enzymes for conversion of polysaccharides into lower sugars as well as sustained conversion of the lower sugars into fresh cells and ethanol.

In another embodiment, the level of a medium component is maintained at a desired level by adding additional medium component as the component is consumed or taken up by the organism. Examples of medium components included, but are not limited to, carbon substrate, nitrogen substrate, vitamins, minerals, growth factors, cofactors, and biocatalysts. The medium component can be added continuously or at regular or irregular intervals. In some embodiments, additional medium component is added prior to the complete depletion of the medium component in the medium. In some embodiments, complete depletion can effectively be used, for example to initiate different metabolic pathways, to simplify downstream operations, or for other reasons as well. In some embodiments, the medium component level is allowed to vary by about 10% around a midpoint, in some embodiments, it is allowed to vary by about 30% around a midpoint, and in some embodiments, it is allowed to vary by 60% or more around a midpoint. Operation in some embodiments will maintain the medium component level by allowing the medium component to be depleted to an appropriate level, followed by increasing the medium component level to another appropriate level. In one embodiment, a medium component, such as vitamin, is added at two different time points during fermentation process. For example, one-half of a total amount of vitamin is added at the beginning of fermentation and the other half is added at midpoint of fermentation.

In another embodiment, the nitrogen level is maintained at a desired level by adding additional nitrogen-containing material as nitrogen is consumed or taken up by the organism. The nitrogen-containing material can be added continuously or at regular or irregular intervals. In some embodiments, additional nitrogen-containing material is added prior to the complete depletion of the nitrogen available in the medium. In some embodiments, complete depletion can effectively be used, for example to initiate different metabolic pathways, to simplify downstream operations, or for other reasons as well. In some embodiments, the nitrogen level (as measured by the grams of actual nitrogen in the nitrogen-containing material per liter of broth) is allowed to vary by about 10% around a midpoint, in some embodiments, it is allowed to vary by about 30% around a midpoint, and in some embodiments, it is allowed to vary by 60% or more around a midpoint. Operation in some embodiments will maintain the nitrogen level by allowing the nitrogen to be depleted to an appropriate level, followed by increasing the nitrogen level to another appropriate level. Useful nitrogen levels include levels of about 5 to about 10 g/L. In one embodiment levels of about 1 to about 12 g/L can also be usefully employed. In another embodiment levels, such as about 0.5, 0.1 g/L or even lower, and higher levels, such as about 20, 30 g/L or even higher are used. In another embodiment a useful nitrogen level is about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22 23, 24, 25, 26, 27, 28, 29 or 30 g/L. Such nitrogen levels can facilitate the production of fresh cells and of hydrolytic enzymes. Increasing the level of nitrogen can lead to higher levels of enzymes and/or greater production of cells, and result in higher productivity of desired products. Nitrogen can be supplied as a simple nitrogen-containing material, such as an ammonium compounds (e.g. ammonium sulfate, ammonium hydroxide, ammonia, ammonium nitrate, or any other compound or mixture containing an ammonium moiety), nitrate or nitrite compounds (e.g. potassium, sodium, ammonium, calcium, or other compound or mixture containing a nitrate or nitrite moiety), or as a more complex nitrogen-containing material, such as amino acids, proteins, hydrolyzed protein, hydrolyzed yeast, yeast extract, dried brewer's yeast, yeast hydrolysates, soy protein, hydrolyzed soy protein, fermentation products, and processed or corn steep powder or unprocessed protein-rich vegetable or animal matter, including those derived from bean, seeds, soy, legumes, nuts, milk, pig, cattle, mammal, fish, as well as other parts of plants and other types of animals. Nitrogen-containing materials useful in various embodiments also include materials that contain a nitrogen-containing material, including, but not limited to mixtures of a simple or more complex nitrogen-containing material mixed with a carbon source, another nitrogen-containing material, or other nutrients or non-nutrients, and AFEX treated plant matter.

In another embodiment, the carbon level is maintained at a desired level by adding sugar compounds or material containing sugar compounds (“Sugar-Containing Material”) as sugar is consumed or taken up by the organism. The sugar-containing material can be added continuously or at regular or irregular intervals. In some embodiments, additional sugar-containing material is added prior to the complete depletion of the sugar compounds available in the medium. In some embodiments, complete depletion can effectively be used, for example to initiate different metabolic pathways, to simplify downstream operations, or for other reasons as well. In some embodiments, the carbon level (as measured by the grams of sugar present in the sugar-containing material per liter of broth) is allowed to vary by about 10% around a midpoint, in some embodiments, it is allowed to vary by about 30% around a midpoint, and in some embodiments, it is allowed to vary by 60% or more around a midpoint. Operation in some embodiments will maintain the carbon level by allowing the carbon to be depleted to an appropriate level, followed by increasing the carbon level to another appropriate level. In some embodiments, the carbon level can be maintained at a level of about 5 to about 120 g/L. However, levels of about 30 to about 100 g/L can also be usefully employed as well as levels of about 60 to about 80 g/L. In one embodiments, the carbon level is maintained at greater than 25 g/L for a portion of the culturing. In another embodiment, the carbon level is maintained at about 5 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, 100 g/L, 101 g/L, 102 g/L, 103 g/L, 104 g/L, 105 g/L, 106 g/L, 107 g/L, 108 g/L, 109 g/L, 110 g/L, 111 g/L, 112 g/L, 113 g/L, 114 g/L, 115 g/L, 116 g/L, 117 g/L, 118 g/L, 119 g/L, 120 g/L, 121 g/L, 122 g/L, 123 g/L, 124 g/L, 125 g/L, 126 g/L, 127 g/L, 128 g/L, 129 g/L, 130 g/L, 131 g/L, 132 g/L, 133 g/L, 134 g/L, 135 g/L, 136 g/L, 137 g/L, 138 g/L, 139 g/L, 140 g/L, 141 g/L, 142 g/L, 143 g/L, 144 g/L, 145 g/L, 146 g/L, 147 g/L, 148 g/L, 149 g/L, or 150 g/L.

The carbon substrate, like the nitrogen substrate, is necessary for cell production and enzyme production, but unlike the nitrogen substrate, it serves as the raw material for ethanol. Frequently, more carbon substrate can lead to greater production of ethanol.

In another embodiment, it can be advantageous to operate with the carbon level and nitrogen level related to each other for at least a portion of the fermentation time. In one embodiment, the ratio of carbon to nitrogen is maintained within a range of about 30:1 to about 10:1. In another embodiment, the ratio of carbon nitrogen is maintained from about 20:1 to about 10:1 or more preferably from about 15:1 to about 10:1. In another embodiment the ratio of carbon nitrogen is about 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or 1:1.

Maintaining the ratio of carbon and nitrogen ratio within particular ranges can result in benefits to the operation such as the rate of hydrolysis of carbon substrate, which depends on the amount of carbon substrate and the amount and activity of enzymes present, being balanced to the rate of ethanol production. Such balancing can be important, for example, due to the possibility of inhibition of cellular activity due to the presence of a high concentration of low molecular weight saccharides, and the need to maintain enzymatic hydrolytic activity throughout the period where longer chain saccharides are present and available for hydrolysis. Balancing the carbon to nitrogen ratio can, for example, facilitate the sustained production of these enzymes such as to replace those which have lost activity.

In another embodiment, the amount and/or timing of carbon, nitrogen, or other medium component addition can be related to measurements taken during the fermentation. For example, the amount of monosaccharides present, the amount of insoluble polysaccharide present, the polysaccharase activity, the amount of ethanol present, the amount of cellular material (for example, packed cell volume, dry cell weight, etc.) and/or the amount of nitrogen (for example, nitrate, nitrite, ammonia, urea, proteins, amino acids, etc.) present can be measured. The concentration of the particular species, the total amount of the species present in the fermentor, the number of hours the fermentation has been running, and the volume of the fermentor can be considered. In various embodiments, these measurements can be compared to each other and/or they can be compared to previous measurements of the same parameter previously taken from the same fermentation or another fermentation. Adjustments to the amount of a medium component can be accomplished such as by changing the flow rate of a stream containing that component or by changing the frequency of the additions for that component. In one embodiment, the amount of polysaccharide can be reduced when the monosaccharides level increases faster than the ethanol level increases. In another embodiment, the amount of polysaccharide can be increased when the amount or level of monosaccharides decreases while the ethanol production approximately remains steady. In another embodiment, the amount of nitrogen can be increased when the monosaccharides level increases faster than the viable cell level. The amount of polysaccharide can also be increased when the cell production increases faster than the ethanol production. In another embodiment the amount of nitrogen can be increased when the enzyme activity level decreases.

In another embodiment, different levels or complete depletion of a medium component can effectively be used, for example to initiate different metabolic pathways or to change the yield of the different products of the fermentation process. For instance, different levels or complete depletion of a medium component can effectively be used to increase the ethanol yield and productivity, to improve carbon utilization (e.g., g ethanol/g sugar fermented) and reduced acid production (e.g., g acid/g ethanol and g acid/g sugar fermented). In some embodiments, different levels or complete depletion of nitrogen can effectively be used to increase the ethanol yield and productivity, to improve carbon utilization (e.g., g ethanol/g sugar fermented) and reduced acid production (e.g., g acid/g ethanol and g acid/g sugar fermented). In some embodiments, different levels or complete depletion of carbon can effectively be used to increase the ethanol yield and productivity, to improve carbon utilization (e.g., g ethanol/g sugar fermented) and reduced acid production (e.g., g acid/g ethanol and g acid/g sugar fermented). In some embodiments, the ratio of carbon level to nitrogen level for at least a portion of the fermentation time can effectively be used to increase the ethanol yield and productivity, to improve carbon utilization (e.g., g ethanol/g sugar fermented) and reduced acid production (e.g., g acid/g ethanol and g acid/g sugar fermented).

In another embodiment, a fed batch operation can be employed, wherein medium components and/or fresh cells are added during the fermentation without removal of a portion of the broth for harvest prior to the end of the fermentation. In one embodiment a fed-batch process is based on feeding a growth limiting nutrient medium to a culture of microorganisms. In one embodiment the feed medium is highly concentrated to avoid dilution of the bioreactor. In another embodiment the controlled addition of the nutrient directly affects the growth rate of the culture and avoids overflow metabolism such as the formation of side metabolites. In one embodiment the growth limiting nutrient is a nitrogen source or a saccharide source.

In another embodiment, a modified fed batch operation can be employed wherein a portion of the broth is harvested at discrete times. Such a modified fed batch operation can be advantageously employed when, for example, very long fermentation cycles are employed. Under very long fermentation conditions, the volume of liquid inside the fermentor increases. In order to operate for very long periods, it can be advantageous to partially empty the fermentor, for example, when the volume is nearly full. A partial harvest of broth followed by supplementation with fresh medium ingredients, such as with a fed batch operation, can improve fermentor utilization and can facilitate higher plant throughputs due to a reduction in the time for tasks such as cleaning and sterilization of equipment. When the “partial harvest” type of operation is employed, the fermentation can be seeded with the broth that remains in the fermentor, or with fresh inoculum, or with a mixture of the two. In addition, broth can be recycled for use as fresh inoculum either alone or in combination with other fresh inoculum.

In some embodiments, a fed batch operation can be employed, wherein medium components and/or fresh cells are added during the fermentation when the hydrolytic activity of the broth has decreased. In some embodiments, medium components and/or fresh cells are added during the fermentation when the hydrolytic activity of the broth has decreased about 5%, 10%, 15%, 20%, 25%, 30%, 35%, 40%, 45%, 50%, 55%, 60%, 65%, 75%, 80%, 85%, 90%, 95%, or 100%.

While the Q microbe can be used in long or short fermentation cycles, it is particularly well-suited for long fermentation cycles and for use in fermentations with partial harvest, self-seeding, and broth recycle operations due to the anaerobic conditions of the fermentation, the presence of alcohol, the fast growth rate of the organism, and, in some embodiments, the use of a solid carbon substrate, whether or not resulting in low sugar concentrations in the broth.

In another embodiment, a fermentation to produce ethanol is performed by culturing a strain of the Q microbe in a medium having a high concentration of one or more carbon sources, and/or augmenting the culture with addition of fresh cells of Q microbe during the course of the fermentation. The resulting production of ethanol can be up to 1-fold, 2-fold, 3-fold, 4-fold, 5-fold, 6-fold, 7-fold, 8-fold, 9-fold, and in some cases up to 10-fold and higher in volumetric productivity than a batch process and achieve a carbon conversion efficiency approaching the theoretical maximum. The theoretical maximum can vary with the substrate and product. For example, the generally accepted maximum efficiency for conversion of glucose to ethanol is 0.51 g ethanol/g glucose. In one embodiment the Q microbe can produce about 40-100% of a theoretical maximum yield of ethanol. In another embodiment, the Q microbe can produce up to about 40% of the theoretical maximum yield of ethanol. In another embodiment, the Q microbe can produce up to about 50% of the theoretical maximum yield of ethanol. In another embodiment, the Q microbe can produce about 70% of the theoretical maximum yield of ethanol. In another embodiment, the Q microbe can produce about 90% of the theoretical maximum yield of ethanol. In another embodiment, the Q microbe can produce about 95% of the theoretical maximum yield of ethanol. In another embodiment, the Q microbe can produce about 95% of the theoretical maximum yield of ethanol. In another embodiment, the Q microbe can produce about 99% of the theoretical maximum yield of ethanol. In another embodiment, the Q microbe can produce about 100% of the theoretical maximum yield of ethanol. In one embodiment a Q microbe can produce up to about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.99%, or 100% of a theoretical maximum yield of ethanol.

The Q microbe cells used for the seed inoculum or for cell augmentation can be prepared or treated in ways that relate to their ability to produce enzymes useful for hydrolyzing the components of the production medium. For example, in one embodiment, the Q microbe cells can produce useful enzymes after they are transferred to the production medium or production fermentor. In another embodiment, the Q microbe cells can have already produced useful enzymes prior to transfer to the production medium or the production fermentor. In another embodiment, the Q microbe cells can be ready to produce useful enzymes once transferred to the production medium or the production fermentor, or the Q microbe cells can have some combination of these enzyme production characteristics. In one embodiment, the seed can be grown initially in a medium containing a simple sugar source, such as corn syrup, and then transitioned to the production medium carbon source prior to transfer to the production medium. In another embodiment, the seed is grown on a combination of simple sugars and production medium carbon source prior to transfer to the production medium. In another embodiment, the seed is grown on the production medium carbon source from the start. In another embodiment, the seed is grown on one production medium carbon source and then transitioned to another production medium carbon source prior to transfer to the production medium. In another embodiment, the seed is grown on a combination of production medium carbon sources prior to transfer to the production medium. In another embodiment, the seed is grown on a carbon source that favors production of hydrolytic enzymes with activity toward the components of the production medium.

In another embodiment, a fermentation to produce ethanol is performed by culturing a strain of the Q microorganism and adding fresh medium components and fresh Q microbe cells while the cells in the fermentor are growing. Medium components, such as carbon, nitrogen, and combinations of these, can be added as disclosed herein, as well as other nutrients, including vitamins, factors, cofactors, enzymes, minerals, salts, and such, sufficient to maintain an effective level of these nutrients in the medium. The medium and Q microbe cells can be added simultaneously, or one at a time. In another embodiment, fresh Q microbe cells can be added when hydrolytic enzyme activity decreases, especially when the activity of those hydrolytic enzymes that are more sensitive to the presence of alcohol decreases. After the addition of fresh Q microbe cells, a nitrogen feed or a combination of nitrogen and carbon feed and/or other medium components can be fed, prolonging the enzymatic production or other activity of the cells. In another embodiment, the cells can be added with sufficient carbon and nitrogen to prolong the enzymatic production or other activity of the cells sufficiently until the next addition of fresh cells. In another embodiment, fresh Q microbe cells can be added when both the nitrogen level and carbon level present in the fermentor increase. In another embodiment, fresh Q microbe cells can be added when the viable cell count decreases, especially when the nitrogen level is relatively stable or increasing. In another embodiment, fresh cells can be added when a significant portion of the viable cells are in the process of sporulation, or have sporulated. Appropriate times for adding fresh Q microbe cells can be when the portion of cells in the process of sporulation or have sporulated is about 2% to about 100%, about 10% to about 75%, about 20% to about 50%, or about 25% to about 30% of the cells are in the process of sporulation or have sporulated.

In other embodiments, a fermentation to produce ethanol is performed by culturing recycled cells as inoculum. A higher population density can be used to increase the production of ethanol. Appropriate levels of inoculum include utilizing less than about 0.01% (v/v) or about 0.01% to about 0.1% (v/v), about 0.1% to about 1% (v/v), about 1% to about 3% (v/v), about 3% to about 5% (v/v) or even as high as 10% (v/v) or even higher. Cell content of the inoculum can be measured in various ways, such as by optical density, microscopic analysis, packed cell volume, dry cell weight, DNA analysis, etc. Suitable levels of cells in the inoculum can be about 0.01 g/mL to about 0.05 g/mL dry cell weight (DCW), about 0.05 g/mL to about 0.1 g/mL dry cell weight (DCW), or about 0.1 g/mL to about 0.3 g/mL dry cell weight (DCW). The total amount of cells inoculated into a fermentation medium can be determined by relating the level of cells, such as determined by dry cell weight or other appropriate means, and the level of inoculum. Preferred total amounts of cells include utilizing about 0.0001 to about 0.001 g dry cells per ml broth, about 0.001 to about 0.01 g dry cells per ml broth, or about 0.01 to about 0.03 g dry cells per ml broth, however, in some cases total amounts higher or lower can be used. Higher ethanol titers can be achieved by such techniques as varying the amount of recycled cells; varying the number of times cells are recycled; varying a medium component level (e.g. carbon and nitrogen levels, separately or in a coordinated fashion), such as by the means described herein; and varying a medium component source (e.g. carbon and/or nitrogen source), such as is described herein.

Through techniques including these, high ethanol concentrations can be achieved. In one embodiment an ethanol concentration that can be achieved by methods described herein that is about 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, 100 g/L, 101 g/L, 102 g/L, 103 g/L, 104 g/L, 105 g/L, 106 g/L, 107 g/L, 108 g/L, 109 g/L, 110 g/L, 111 g/L, 112 g/L, 113 g/L, 114 g/L, 115 g/L, 116 g/L, 117 g/L, 118 g/L, 119 g/L, 120 g/L, 121 g/L, 122 g/L, 123 g/L, 124 g/L, 125 g/L, 126 g/L, 127 g/L, 128 g/L, 129 g/L, 130 g/L, 131 g/L, 132 g/L, 133 g/L, 134 g/L, 135 g/L, 136 g/L, 137 g/L, 138 g/L, 139 g/L, 140 g/L, 141 g/L, 142 g/L, 143 g/L, 144 g/L, 145 g/L, 146 g/L, 147 g/L, 148 g/L, 149 g/L, 150 g/L, 151 g/L, 152 g/L, 153 g/L, 154 g/L, 155 g/L, 156 g/L, 157 g/L, 158 g/L, 159 g/L, 160 g/L, 161 g/L, 162 g/L, 163 g/L, 164 g/L, 165 g/L, 166 g/L, 167 g/L, 168 g/L, 169 g/L, 170 g/L, 171 g/L, 172 g/L, 173 g/L, 174 g/L, 175 g/L, 176 g/L, 177 g/L, 178 g/L, 179 g/L, 180 g/L, or 181 g/L.

In another embodiment, a fermentation to produce ethanol is performed by culturing a strain of the Q microorganism and adding recycled Q microbe cells while the cells in the fermentor are cell expansion stage (e.g. seed stage) and/or the final fermentation stage of a fermentation. Without intending to be limited to any theory the results described herein indicate that the recycled cells have a tolerance of higher ethanol concentrations and the ability to grow in such an environment. Thus, such a tolerance and ability can be useful for situations such as the cell expansion stage (e.g. seed stage) and the final fermentation stage of a fermentation where these concentrations of ethanol are present, including ethanol production fermentations, or for the production of other products in the presence of these concentrations of ethanol.

### Medium Compositions

In various embodiments, particular medium components can have beneficial effects on the performance of the fermentation, such as increasing the titer of desired products, or increasing the rate that the desired products are produced. Specific compounds can be supplied as a specific, pure ingredient, such as a particular amino acid, or it can be supplied as a component of a more complex ingredient, such as using a microbial, plant or animal product as a medium ingredient to provide a particular amino acid, promoter, cofactor, or other beneficial compound. In some cases, the particular compound supplied in the medium ingredient can be combined with other compounds by the organism resulting in a fermentation-beneficial compound. One example of this situation would be where a medium ingredient provides a specific amino acid which the organism uses to make an enzyme beneficial to the fermentation. Other examples can include medium components that are used to generate growth or product promoters, etc. In such cases, it can be possible to obtain a fermentation-beneficial result by supplementing the enzyme, promoter, growth factor, etc. or by adding the precursor. In some situations, the specific mechanism whereby the medium component benefits the fermentation is not known, only that a beneficial result is achieved.

In one embodiment, beneficial fermentation results can be achieved by adding yeast extract. A typical composition for yeast extract is shown in Table 8. The addition of the yeast extract can result in increased ethanol titer in batch fermentation, improved productivity and reduced production of side products such as organic acids. In one embodiment beneficial results with yeast extract can be achieved in the methods of the embodiments at usage levels of about 0.5 to about 50 g/L, about 5 to about 30 g/L, or about 10 to about 30 g/L. In another embodiment the yeast extract is used at level about 0.5 g/L, 0.6 g/L, 0.7 g/L, 0.8 g/L, 0.9 g/L, 1 g/L, 1.1 g/L, 1.2 g/L, 1.3 g/L, 1.4 g/L, 1.5 g/L, 1.6 g/L, 1.7 g/L, 1.8 g/L, 1.9 g/L, 2 g/L, 2.1 g/L, 2.2 g/L, 2.3 g/L, 2.4 g/L, 2.5 g/L, 2.6 g/L, 2.7 g/L, 2.8 g/L, 2.9 g/L, 3 g/L, 3.1 g/L, 3.2 g/L, 3.3 g/L, 3.4 g/L, 3.5 g/L, 3.6 g/L, 3.7 g/L, 3.8 g/L, 3.9 g/L, 4 g/L, 4.1 g/L, 4.2 g/L, 4.3 g/L, 4.4 g/L, 4.5 g/L, 4.6 g/L, 4.7 g/L, 4.8 g/L, 4.9 g/L, 5 g/L, 5.1 g/L, 5.2 g/L, 5.3 g/L, 5.4 g/L, 5.5 g/L, 5.6 g/L, 5.7 g/L, 5.8 g/L, 5.9 g/L, 6 g/L, 6.1 g/L, 6.2 g/L, 6.3 g/L, 6.4 g/L, 6.5 g/L, 6.6 g/L, 6.7 g/L, 6.8 g/L, 6.9 g/L, 7 g/L, 7.1 g/L, 7.2 g/L, 7.3 g/L, 7.4 g/L, 7.5 g/L, 7.6 g/L, 7.7 g/L, 7.8 g/L, 7.9 g/L, 8 g/L, 8.1 g/L, 8.2 g/L, 8.3 g/L, 8.4 g/L, 8.5 g/L, 8.6 g/L, 8.7 g/L, 8.8 g/L, 8.9 g/L, 9 g/L, 9.1 g/L, 9.2 g/L, 9.3 g/L, 9.4 g/L, 9.5 g/L, 9.6 g/L, 9.7 g/L, 9.8 g/L, 9.9 g/L, 10 g/L, 10.1 g/L, 10.2 g/L, 10.3 g/L, 10.4 g/L, 10.5 g/L, 10.6 g/L, 10.7 g/L, 10.8 g/L, 10.9 g/L, 11 g/L, 11.1 g/L, 11.2 g/L, 11.3 g/L, 11.4 g/L, 11.5 g/L, 11.6 g/L, 11.7 g/L, 11.8 g/L, 11.9 g/L, 12 g/L, 12.1 g/L, 12.2 g/L, 12.3 g/L, 12.4 g/L, 12.5 g/L, 12.6 g/L, 12.7 g/L, 12.8 g/L, 12.9 g/L, 13 g/L, 13.1 g/L, 13.2 g/L, 13.3 g/L, 13.4 g/L, 13.5 g/L, 13.6 g/L, 13.7 g/L, 13.8 g/L, 13.9 g/L, 14 g/L, 14.1 g/L, 14.2 g/L, 14.3 g/L, 14.4 g/L, 14.5 g/L, 14.6 g/L, 14.7 g/L, 14.8 g/L, 14.9 g/L, 15 g/L, 15.1 g/L, 15.2 g/L, 15.3 g/L, 15.4 g/L, 15.5 g/L, 15.6 g/L, 15.7 g/L, 15.8 g/L, 15.9 g/L, 16 g/L, 16.1 g/L, 16.2 g/L, 16.3 g/L, 16.4 g/L, 16.5 g/L, 16.6 g/L, 16.7 g/L, 16.8 g/L, 16.9 g/L, 17 g/L, 17.1 g/L, 17.2 g/L, 17.3 g/L, 17.4 g/L, 17.5 g/L, 17.6 g/L, 17.7 g/L, 17.8 g/L, 17.9 g/L, 18 g/L, 18.1 g/L, 18.2 g/L, 18.3 g/L, 18.4 g/L, 18.5 g/L, 18.6 g/L, 18.7 g/L, 18.8 g/L, 18.9 g/L, 19 g/L, 19.1 g/L, 19.2 g/L, 19.3 g/L, 19.4 g/L, 19.5 g/L, 19.6 g/L, 19.7 g/L, 19.8 g/L, 19.9 g/L, 20 g/L, 20.1 g/L, 20.2 g/L, 20.3 g/L, 20.4 g/L, 20.5 g/L, 20.6 g/L, 20.7 g/L, 20.8 g/L, 20.9 g/L, 21 g/L, 21.1 g/L, 21.2 g/L, 21.3 g/L, 21.4 g/L, 21.5 g/L, 21.6 g/L, 21.7 g/L, 21.8 g/L, 21.9 g/L, 22 g/L, 22.1 g/L, 22.2 g/L, 22.3 g/L, 22.4 g/L, 22.5 g/L, 22.6 g/L, 22.7 g/L, 22.8 g/L, 22.9 g/L, 23 g/L, 23.1 g/L, 23.2 g/L, 23.3 g/L, 23.4 g/L, 23.5 g/L, 23.6 g/L, 23.7 g/L, 23.8 g/L, 23.9 g/L, 24 g/L, 24.1 g/L, 24.2 g/L, 24.3 g/L, 24.4 g/L, 24.5 g/L, 24.6 g/L, 24.7 g/L, 24.8 g/L, 24.9 g/L, 25 g/L, 25.1 g/L, 25.2 g/L, 25.3 g/L, 25.4 g/L, 25.5 g/L, 25.6 g/L, 25.7 g/L, 25.8 g/L, 25.9 g/L, 26 g/L, 26.1 g/L, 26.2 g/L, 26.3 g/L, 26.4 g/L, 26.5 g/L, 26.6 g/L, 26.7 g/L, 26.8 g/L, 26.9 g/L, 27 g/L, 27.1 g/L, 27.2 g/L, 27.3 g/L, 27.4 g/L, 27.5 g/L, 27.6 g/L, 27.7 g/L, 27.8 g/L, 27.9 g/L, 28 g/L, 28.1 g/L, 28.2 g/L, 28.3 g/L, 28.4 g/L, 28.5 g/L, 28.6 g/L, 28.7 g/L, 28.8 g/L, 28.9 g/L, 29 g/L, 29.1 g/L, 29.2 g/L, 29.3 g/L, 29.4 g/L, 29.5 g/L, 29.6 g/L, 29.7 g/L, 29.8 g/L, 29.9 g/L, 30 g/L, 30.1 g/L, 30.2 g/L, 30.3 g/L, 30.4 g/L, 30.5 g/L, 30.6 g/L, 30.7 g/L, 30.8 g/L, 30.9 g/L, 31 g/L, 31.1 g/L, 31.2 g/L, 31.3 g/L, 31.4 g/L, 31.5 g/L, 31.6 g/L, 31.7 g/L, 31.8 g/L, 31.9 g/L, 32 g/L, 32.1 g/L, 32.2 g/L, 32.3 g/L, 32.4 g/L, 32.5 g/L, 32.6 g/L, 32.7 g/L, 32.8 g/L, 32.9 g/L, 33 g/L, 33.1 g/L, 33.2 g/L, 33.3 g/L, 33.4 g/L, 33.5 g/L, 33.6 g/L, 33.7 g/L, 33.8 g/L, 33.9 g/L, 34 g/L, 34.1 g/L, 34.2 g/L, 34.3 g/L, 34.4 g/L, 34.5 g/L, 34.6 g/L, 34.7 g/L, 34.8 g/L, 34.9 g/L, 35 g/L, 35.1 g/L, 35.2 g/L, 35.3 g/L, 35.4 g/L, 35.5 g/L, 35.6 g/L, 35.7 g/L, 35.8 g/L, 35.9 g/L, 36 g/L, 36.1 g/L, 36.2 g/L, 36.3 g/L, 36.4 g/L, 36.5 g/L, 36.6 g/L, 36.7 g/L, 36.8 g/L, 36.9 g/L, 37 g/L, 37.1 g/L, 37.2 g/L, 37.3 g/L, 37.4 g/L, 37.5 g/L, 37.6 g/L, 37.7 g/L, 37.8 g/L, 37.9 g/L, 38 g/L, 38.1 g/L, 38.2 g/L, 38.3 g/L, 38.4 g/L, 38.5 g/L, 38.6 g/L, 38.7 g/L, 38.8 g/L, 38.9 g/L, 39 g/L, 39.1 g/L, 39.2 g/L, 39.3 g/L, 39.4 g/L, 39.5 g/L, 39.6 g/L, 39.7 g/L, 39.8 g/L, 39.9 g/L, 40 g/L, 40.1 g/L, 40.2 g/L, 40.3 g/L, 40.4 g/L, 40.5 g/L, 40.6 g/L, 40.7 g/L, 40.8 g/L, 40.9 g/L, 41 g/L, 41.1 g/L, 41.2 g/L, 41.3 g/L, 41.4 g/L, 41.5 g/L, 41.6 g/L, 41.7 g/L, 41.8 g/L, 41.9 g/L, 42 g/L, 42.1 g/L, 42.2 g/L, 42.3 g/L, 42.4 g/L, 42.5 g/L, 42.6 g/L, 42.7 g/L, 42.8 g/L, 42.9 g/L, 43 g/L, 43.1 g/L, 43.2 g/L, 43.3 g/L, 43.4 g/L, 43.5 g/L, 43.6 g/L, 43.7 g/L, 43.8 g/L, 43.9 g/L, 44 g/L, 44.1 g/L, 44.2 g/L, 44.3 g/L, 44.4 g/L, 44.5 g/L, 44.6 g/L, 44.7 g/L, 44.8 g/L, 44.9 g/L, 45 g/L, 45.1 g/L, 45.2 g/L, 45.3 g/L, 45.4 g/L, 45.5 g/L, 45.6 g/L, 45.7 g/L, 45.8 g/L, 45.9 g/L, 46 g/L, 46.1 g/L, 46.2 g/L, 46.3 g/L, 46.4 g/L, 46.5 g/L, 46.6 g/L, 46.7 g/L, 46.8 g/L, 46.9 g/L, 47 g/L, 47.1 g/L, 47.2 g/L, 47.3 g/L, 47.4 g/L, 47.5 g/L, 47.6 g/L, 47.7 g/L, 47.8 g/L, 47.9 g/L, 48 g/L, 48.1 g/L, 48.2 g/L, 48.3 g/L, 48.4 g/L, 48.5 g/L, 48.6 g/L, 48.7 g/L, 48.8 g/L, 48.9 g/L, 49 g/L, 49.1 g/L, 49.2 g/L, 49.3 g/L, 49.4 g/L, 49.5 g/L, 49.6 g/L, 49.7 g/L, 49.8 g/L, 49.9 g/L or 50 g/L.

The yeast extract can also be fed throughout the course of the entire fermentation or a portion of the fermentation, continuously or delivered at intervals. In one embodiment usage levels include maintaining a nitrogen concentration of about 0.05 g/L to about 3 g/L (as nitrogen), where at least a portion of the nitrogen is supplied from corn steep powder; or about 0.3 g/L to 1.3 g/L; or 0.4 g/L to about 0.9 g/L. In another embodiment the nitrogen concentration is about 0.05 g/L, 0.06 g/L, 0.07 g/L, 0.08 g/L, 0.09 g/L, 0.1 g/L, 0.11 g/L, 0.12 g/L, 0.13 g/L, 0.14 g/L, 0.15 g/L, 0.16 g/L, 0.17 g/L, 0.18 g/L, 0.19 g/L, 0.2 g/L, 0.21 g/L, 0.22 g/L, 0.23 g/L, 0.24 g/L, 0.25 g/L, 0.26 g/L, 0.27 g/L, 0.28 g/L, 0.29 g/L, 0.3 g/L, 0.31 g/L, 0.32 g/L, 0.33 g/L, 0.34 g/L, 0.35 g/L, 0.36 g/L, 0.37 g/L, 0.38 g/L, 0.39 g/L, 0.4 g/L, 0.41 g/L, 0.42 g/L, 0.43 g/L, 0.44 g/L, 0.45 g/L, 0.46 g/L, 0.47 g/L, 0.48 g/L, 0.49 g/L, 0.5 g/L, 0.51 g/L, 0.52 g/L, 0.53 g/L, 0.54 g/L, 0.55 g/L, 0.56 g/L, 0.57 g/L, 0.58 g/L, 0.59 g/L, 0.6 g/L, 0.61 g/L, 0.62 g/L, 0.63 g/L, 0.64 g/L, 0.65 g/L, 0.66 g/L, 0.67 g/L, 0.68 g/L, 0.69 g/L, 0.7 g/L, 0.71 g/L, 0.72 g/L, 0.73 g/L, 0.74 g/L, 0.75 g/L, 0.76 g/L, 0.77 g/L, 0.78 g/L, 0.79 g/L, 0.8 g/L, 0.81 g/L, 0.82 g/L, 0.83 g/L, 0.84 g/L, 0.85 g/L, 0.86 g/L, 0.87 g/L, 0.88 g/L, 0.89 g/L, 0.9 g/L, 0.91 g/L, 0.92 g/L, 0.93 g/L, 0.94 g/L, 0.95 g/L, 0.96 g/L, 0.97 g/L, 0.98 g/L, 0.99 g/L, 1 g/L, 1.01 g/L, 1.02 g/L, 1.03 g/L, 1.04 g/L, 1.05 g/L, 1.06 g/L, 1.07 g/L, 1.08 g/L, 1.09 g/L, 1.1 g/L, 1.11 g/L, 1.12 g/L, 1.13 g/L, 1.14 g/L, 1.15 g/L, 1.16 g/L, 1.17 g/L, 1.18 g/L, 1.19 g/L, 1.2 g/L, 1.21 g/L, 1.22 g/L, 1.23 g/L, 1.24 g/L, 1.25 g/L, 1.26 g/L, 1.27 g/L, 1.28 g/L, 1.29 g/L, 1.3 g/L, 1.31 g/L, 1.32 g/L, 1.33 g/L, 1.34 g/L, 1.35 g/L, 1.36 g/L, 1.37 g/L, 1.38 g/L, 1.39 g/L, 1.4 g/L, 1.41 g/L, 1.42 g/L, 1.43 g/L, 1.44 g/L, 1.45 g/L, 1.46 g/L, 1.47 g/L, 1.48 g/L, 1.49 g/L, 1.5 g/L, 1.51 g/L, 1.52 g/L, 1.53 g/L, 1.54 g/L, 1.55 g/L, 1.56 g/L, 1.57 g/L, 1.58 g/L, 1.59 g/L, 1.6 g/L, 1.61 g/L, 1.62 g/L, 1.63 g/L, 1.64 g/L, 1.65 g/L, 1.66 g/L, 1.67 g/L, 1.68 g/L, 1.69 g/L, 1.7 g/L, 1.71 g/L, 1.72 g/L, 1.73 g/L, 1.74 g/L, 1.75 g/L, 1.76 g/L, 1.77 g/L, 1.78 g/L, 1.79 g/L, 1.8 g/L, 1.81 g/L, 1.82 g/L, 1.83 g/L, 1.84 g/L, 1.85 g/L, 1.86 g/L, 1.87 g/L, 1.88 g/L, 1.89 g/L, 1.9 g/L, 1.91 g/L, 1.92 g/L, 1.93 g/L, 1.94 g/L, 1.95 g/L, 1.96 g/L, 1.97 g/L, 1.98 g/L, 1.99 g/L, 2 g/L, 2.01 g/L, 2.02 g/L, 2.03 g/L, 2.04 g/L, 2.05 g/L, 2.06 g/L, 2.07 g/L, 2.08 g/L, 2.09 g/L, 2.1 g/L, 2.11 g/L, 2.12 g/L, 2.13 g/L, 2.14 g/L, 2.15 g/L, 2.16 g/L, 2.17 g/L, 2.18 g/L, 2.19 g/L, 2.2 g/L, 2.21 g/L, 2.22 g/L, 2.23 g/L, 2.24 g/L, 2.25 g/L, 2.26 g/L, 2.27 g/L, 2.28 g/L, 2.29 g/L, 2.3 g/L, 2.31 g/L, 2.32 g/L, 2.33 g/L, 2.34 g/L, 2.35 g/L, 2.36 g/L, 2.37 g/L, 2.38 g/L, 2.39 g/L, 2.4 g/L, 2.41 g/L, 2.42 g/L, 2.43 g/L, 2.44 g/L, 2.45 g/L, 2.46 g/L, 2.47 g/L, 2.48 g/L, 2.49 g/L, 2.5 g/L, 2.51 g/L, 2.52 g/L, 2.53 g/L, 2.54 g/L, 2.55 g/L, 2.56 g/L, 2.57 g/L, 2.58 g/L, 2.59 g/L, 2.6 g/L, 2.61 g/L, 2.62 g/L, 2.63 g/L, 2.64 g/L, 2.65 g/L, 2.66 g/L, 2.67 g/L, 2.68 g/L, 2.69 g/L, 2.7 g/L, 2.71 g/L, 2.72 g/L, 2.73 g/L, 2.74 g/L, 2.75 g/L, 2.76 g/L, 2.77 g/L, 2.78 g/L, 2.79 g/L, 2.8 g/L, 2.81 g/L, 2.82 g/L, 2.83 g/L, 2.84 g/L, 2.85 g/L, 2.86 g/L, 2.87 g/L, 2.88 g/L, 2.89 g/L, 2.9 g/L, 2.91 g/L, 2.92 g/L, 2.93 g/L, 2.94 g/L, 2.95 g/L, 2.96 g/L, 2.97 g/L, 2.98 g/L, 2.99 g/L, or 3 g/L.

In one embodiment, beneficial fermentation results can be achieved by adding corn steep powder to the fermentation. In another embodiment a typical composition for corn steep powder is shown in Tables 1-2. The addition of the corn steep powder can result in increased ethanol titer in batch fermentation, improved productivity and reduced production of side products such as organic acids. In another embodiment beneficial results with corn steep powder can be achieved in the methods of the embodiments at usage levels of about 3 to about 20 g/L, about 5 to about 15 g/L, or about 8 to about 12 g/L. In another embodiment beneficial results with steep powder can be achieved at a level of about 3 g/L, 3.1 g/L, 3.2 g/L, 3.3 g/L, 3.4 g/L, 3.5 g/L, 3.6 g/L, 3.7 g/L, 3.8 g/L, 3.9 g/L, 4 g/L, 4.1 g/L, 4.2 g/L, 4.3 g/L, 4.4 g/L, 4.5 g/L, 4.6 g/L, 4.7 g/L, 4.8 g/L, 4.9 g/L, 5 g/L, 5.1 g/L, 5.2 g/L, 5.3 g/L, 5.4 g/L, 5.5 g/L, 5.6 g/L, 5.7 g/L, 5.8 g/L, 5.9 g/L, 6 g/L, 6.1 g/L, 6.2 g/L, 6.3 g/L, 6.4 g/L, 6.5 g/L, 6.6 g/L, 6.7 g/L, 6.8 g/L, 6.9 g/L, 7 g/L, 7.1 g/L, 7.2 g/L, 7.3 g/L, 7.4 g/L, 7.5 g/L, 7.6 g/L, 7.7 g/L, 7.8 g/L, 7.9 g/L, 8 g/L, 8.1 g/L, 8.2 g/L, 8.3 g/L, 8.4 g/L, 8.5 g/L, 8.6 g/L, 8.7 g/L, 8.8 g/L, 8.9 g/L, 9 g/L, 9.1 g/L, 9.2 g/L, 9.3 g/L, 9.4 g/L, 9.5 g/L, 9.6 g/L, 9.7 g/L, 9.8 g/L, 9.9 g/L, 10 g/L, 10.1 g/L, 10.2 g/L, 10.3 g/L, 10.4 g/L, 10.5 g/L, 10.6 g/L, 10.7 g/L, 10.8 g/L, 10.9 g/L, 11 g/L, 11.1 g/L, 11.2 g/L, 11.3 g/L, 11.4 g/L, 11.5 g/L, 11.6 g/L, 11.7 g/L, 11.8 g/L, 11.9 g/L, 12 g/L, 12.1 g/L, 12.2 g/L, 12.3 g/L, 12.4 g/L, 12.5 g/L, 12.6 g/L, 12.7 g/L, 12.8 g/L, 12.9 g/L, 13 g/L, 13.1 g/L, 13.2 g/L, 13.3 g/L, 13.4 g/L, 13.5 g/L, 13.6 g/L, 13.7 g/L, 13.8 g/L, 13.9 g/L, 14 g/L, 14.1 g/L, 14.2 g/L, 14.3 g/L, 14.4 g/L, 14.5 g/L, 14.6 g/L, 14.7 g/L, 14.8 g/L, 14.9 g/L, 15 g/L, 15.1 g/L, 15.2 g/L, 15.3 g/L, 15.4 g/L, 15.5 g/L, 15.6 g/L, 15.7 g/L, 15.8 g/L, 15.9 g/L, 16 g/L, 16.1 g/L, 16.2 g/L, 16.3 g/L, 16.4 g/L, 16.5 g/L, 16.6 g/L, 16.7 g/L, 16.8 g/L, 16.9 g/L, 17 g/L, 17.1 g/L, 17.2 g/L, 17.3 g/L, 17.4 g/L, 17.5 g/L, 17.6 g/L, 17.7 g/L, 17.8 g/L, 17.9 g/L, 18 g/L, 18.1 g/L, 18.2 g/L, 18.3 g/L, 18.4 g/L, 18.5 g/L, 18.6 g/L, 18.7 g/L, 18.8 g/L, 18.9 g/L, 19 g/L, 19.1 g/L, 19.2 g/L, 19.3 g/L, 19.4 g/L, 19.5 g/L, 19.6 g/L, 19.7 g/L, 19.8 g/L, 19.9 g/L, or 20 g/L.

In one embodiment corn steep powder can also be fed throughout the course of the entire fermentation or a portion of the fermentation, continuously or delivered at intervals. In another embodiment usage levels include maintaining a nitrogen concentration of about 0.05 g/L to about 3 g/L (as nitrogen), where at least a portion of the nitrogen is supplied from corn steep powder; about 0.3 g/L to 1.3 g/L; or about 0.4 g/L to about 0.9 g/L. In another embodiment the nitrogen level is about 0.05 g/L, 0.06 g/L, 0.07 g/L, 0.08 g/L, 0.09 g/L, 0.1 g/L, 0.11 g/L, 0.12 g/L, 0.13 g/L, 0.14 g/L, 0.15 g/L, 0.16 g/L, 0.17 g/L, 0.18 g/L, 0.19 g/L, 0.2 g/L, 0.21 g/L, 0.22 g/L, 0.23 g/L, 0.24 g/L, 0.25 g/L, 0.26 g/L, 0.27 g/L, 0.28 g/L, 0.29 g/L, 0.3 g/L, 0.31 g/L, 0.32 g/L, 0.33 g/L, 0.34 g/L, 0.35 g/L, 0.36 g/L, 0.37 g/L, 0.38 g/L, 0.39 g/L, 0.4 g/L, 0.41 g/L, 0.42 g/L, 0.43 g/L, 0.44 g/L, 0.45 g/L, 0.46 g/L, 0.47 g/L, 0.48 g/L, 0.49 g/L, 0.5 g/L, 0.51 g/L, 0.52 g/L, 0.53 g/L, 0.54 g/L, 0.55 g/L, 0.56 g/L, 0.57 g/L, 0.58 g/L, 0.59 g/L, 0.6 g/L, 0.61 g/L, 0.62 g/L, 0.63 g/L, 0.64 g/L, 0.65 g/L, 0.66 g/L, 0.67 g/L, 0.68 g/L, 0.69 g/L, 0.7 g/L, 0.71 g/L, 0.72 g/L, 0.73 g/L, 0.74 g/L, 0.75 g/L, 0.76 g/L, 0.77 g/L, 0.78 g/L, 0.79 g/L, 0.8 g/L, 0.81 g/L, 0.82 g/L, 0.83 g/L, 0.84 g/L, 0.85 g/L, 0.86 g/L, 0.87 g/L, 0.88 g/L, 0.89 g/L, 0.9 g/L, 0.91 g/L, 0.92 g/L, 0.93 g/L, 0.94 g/L, 0.95 g/L, 0.96 g/L, 0.97 g/L, 0.98 g/L, 0.99 g/L, 1 g/L, 1.01 g/L, 1.02 g/L, 1.03 g/L, 1.04 g/L, 1.05 g/L, 1.06 g/L, 1.07 g/L, 1.08 g/L, 1.09 g/L, 1.1 g/L, 1.11 g/L, 1.12 g/L, 1.13 g/L, 1.14 g/L, 1.15 g/L, 1.16 g/L, 1.17 g/L, 1.18 g/L, 1.19 g/L, 1.2 g/L, 1.21 g/L, 1.22 g/L, 1.23 g/L, 1.24 g/L, 1.25 g/L, 1.26 g/L, 1.27 g/L, 1.28 g/L, 1.29 g/L, 1.3 g/L, 1.31 g/L, 1.32 g/L, 1.33 g/L, 1.34 g/L, 1.35 g/L, 1.36 g/L, 1.37 g/L, 1.38 g/L, 1.39 g/L, 1.4 g/L, 1.41 g/L, 1.42 g/L, 1.43 g/L, 1.44 g/L, 1.45 g/L, 1.46 g/L, 1.47 g/L, 1.48 g/L, 1.49 g/L, 1.5 g/L, 1.51 g/L, 1.52 g/L, 1.53 g/L, 1.54 g/L, 1.55 g/L, 1.56 g/L, 1.57 g/L, 1.58 g/L, 1.59 g/L, 1.6 g/L, 1.61 g/L, 1.62 g/L, 1.63 g/L, 1.64 g/L, 1.65 g/L, 1.66 g/L, 1.67 g/L, 1.68 g/L, 1.69 g/L, 1.7 g/L, 1.71 g/L, 1.72 g/L, 1.73 g/L, 1.74 g/L, 1.75 g/L, 1.76 g/L, 1.77 g/L, 1.78 g/L, 1.79 g/L, 1.8 g/L, 1.81 g/L, 1.82 g/L, 1.83 g/L, 1.84 g/L, 1.85 g/L, 1.86 g/L, 1.87 g/L, 1.88 g/L, 1.89 g/L, 1.9 g/L, 1.91 g/L, 1.92 g/L, 1.93 g/L, 1.94 g/L, 1.95 g/L, 1.96 g/L, 1.97 g/L, 1.98 g/L, 1.99 g/L, 2 g/L, 2.01 g/L, 2.02 g/L, 2.03 g/L, 2.04 g/L, 2.05 g/L, 2.06 g/L, 2.07 g/L, 2.08 g/L, 2.09 g/L, 2.1 g/L, 2.11 g/L, 2.12 g/L, 2.13 g/L, 2.14 g/L, 2.15 g/L, 2.16 g/L, 2.17 g/L, 2.18 g/L, 2.19 g/L, 2.2 g/L, 2.21 g/L, 2.22 g/L, 2.23 g/L, 2.24 g/L, 2.25 g/L, 2.26 g/L, 2.27 g/L, 2.28 g/L, 2.29 g/L, 2.3 g/L, 2.31 g/L, 2.32 g/L, 2.33 g/L, 2.34 g/L, 2.35 g/L, 2.36 g/L, 2.37 g/L, 2.38 g/L, 2.39 g/L, 2.4 g/L, 2.41 g/L, 2.42 g/L, 2.43 g/L, 2.44 g/L, 2.45 g/L, 2.46 g/L, 2.47 g/L, 2.48 g/L, 2.49 g/L, 2.5 g/L, 2.51 g/L, 2.52 g/L, 2.53 g/L, 2.54 g/L, 2.55 g/L, 2.56 g/L, 2.57 g/L, 2.58 g/L, 2.59 g/L, 2.6 g/L, 2.61 g/L, 2.62 g/L, 2.63 g/L, 2.64 g/L, 2.65 g/L, 2.66 g/L, 2.67 g/L, 2.68 g/L, 2.69 g/L, 2.7 g/L, 2.71 g/L, 2.72 g/L, 2.73 g/L, 2.74 g/L, 2.75 g/L, 2.76 g/L, 2.77 g/L, 2.78 g/L, 2.79 g/L, 2.8 g/L, 2.81 g/L, 2.82 g/L, 2.83 g/L, 2.84 g/L, 2.85 g/L, 2.86 g/L, 2.87 g/L, 2.88 g/L, 2.89 g/L, 2.9 g/L, 2.91 g/L, 2.92 g/L, 2.93 g/L, 2.94 g/L, 2.95 g/L, 2.96 g/L, 2.97 g/L, 2.98 g/L, 2.99 g/L, or 3 g/L.

In another embodiment, other related products can be used, such as corn steep liquor or corn steep solids. When corn steep liquor is used, the usage rate would be approximately the same as for corn steep solids on a solids basis. In another embodiment, the corn steep powder (or solids or liquor) is added in relation to the amount of carbon substrate that is present or that will be added. When added in this way, beneficial amounts of corn steep powder (or liquor or solids) can include about 1:1 to about 1:6 g/g carbon, about 1:1 to about 1:5 g/g carbon, or about 1:2 to about 1:4 g/g carbon. In another embodiment ratios as high as about 1.5:1 g/g carbon or about 3:1 g/g carbon or as low as about 1:8 g/g carbon or about 1:10 g/g carbon are used. In another embodiment the ratio is 2:1 g/g carbon, 1.9:1 g/g carbon, 1.8:1 g/g carbon, 1.7:1 g/g carbon, 1.6:1 g/g carbon, 1.5:1 g/g carbon, 1.4:1 g/g carbon, 1.3:1 g/g carbon, 1.2:1 g/g carbon, 1.1:1 g/g carbon, 1:1 g/g carbon, 1:1.1 g/g carbon, 1:1.2 g/g carbon, 1:1.3 g/g carbon, 1:1.4 g/g carbon, 1:1.5 g/g carbon, 1:1.6 g/g carbon, 1:1.7 g/g carbon, 1:1.8 g/g carbon, 1:1.9 g/g carbon, 1:2 g/g carbon, 1:2.1 g/g carbon, 1:2.2 g/g carbon, 1:2.3 g/g carbon, 1:2.4 g/g carbon, 1:2.5 g/g carbon, 1:2.6 g/g carbon, 1:2.7 g/g carbon, 1:2.8 g/g carbon, 1:2.9 g/g carbon, 1:3 g/g carbon, 1:3.1 g/g carbon, 1:3.2 g/g carbon, 1:3.3 g/g carbon, 1:3.4 g/g carbon, 1:3.5 g/g carbon, 1:3.6 g/g carbon, 1:3.7 g/g carbon, 1:3.8 g/g carbon, 1:3.9 g/g carbon, 1:4 g/g carbon, 1:4.1 g/g carbon, 1:4.2 g/g carbon, 1:4.3 g/g carbon, 1:4.4 g/g carbon, 1:4.5 g/g carbon, 1:4.6 g/g carbon, 1:4.7 g/g carbon, 1:4.8 g/g carbon, 1:4.9 g/g carbon, 1:5 g/g carbon, 1:5.1 g/g carbon, 1:5.2 g/g carbon, 1:5.3 g/g carbon, 1:5.4 g/g carbon, 1:5.5 g/g carbon, 1:5.6 g/g carbon, 1:5.7 g/g carbon, 1:5.8 g/g carbon, 1:5.9 g/g carbon, 1:6 g/g carbon, 1:6.1 g/g carbon, 1:6.2 g/g carbon, 1:6.3 g/g carbon, 1:6.4 g/g carbon, 1:6.5 g/g carbon, 1:6.6 g/g carbon, 1:6.7 g/g carbon, 1:6.8 g/g carbon, 1:6.9 g/g carbon, 1:7 g/g carbon, 1:7.1 g/g carbon, 1:7.2 g/g carbon, 1:7.3 g/g carbon, 1:7.4 g/g carbon, 1:7.5 g/g carbon, 1:7.6 g/g carbon, 1:7.7 g/g carbon, 1:7.8 g/g carbon, 1:7.9 g/g carbon, 1:8 g/g carbon, 1:8.1 g/g carbon, 1:8.2 g/g carbon, 1:8.3 g/g carbon, 1:8.4 g/g carbon, 1:8.5 g/g carbon, 1:8.6 g/g carbon, 1:8.7 g/g carbon, 1:8.8 g/g carbon, 1:8.9 g/g carbon, 1:9 g/g carbon, 1:9.1 g/g carbon, 1:9.2 g/g carbon, 1:9.3 g/g carbon, 1:9.4 g/g carbon, 1:9.5 g/g carbon, 1:9.6 g/g carbon, 1:9.7 g/g carbon, 1:9.8 g/g carbon, 1:9.9 g/g carbon, or 1:10 g/g carbon.

TABLE 5
Compositional characteristics of corn steep powder
(source (except as noted): product datasheet for
spray dried corn steep liquor, Roquette, Solulys 095E).

| Parameter | Value |
|---|---|
| Loss on drying | 5.5% maximum |
| pH in solution | 3.9-4.5 |
| total acidity (as lactic acid) | 14-20% |
| reducing sugars | 1.5% maximum |
| amino nitrogen | 1.5-3.5% |
| total nitrogen | 7.0-8.5% |
| Ash | 13.5-17.5% |
| phosphorus (as P) | 2.4-3.2% |
| protein content (N × 6.25) | 48% (approximately) |
| Phytic acid (dry weight basis) | 8% (source: WO1997035489 19971002; |
| | A Process for Obtaining Phytic |
| | Acid and Lactic Acid) |

TABLE 6
Typical amino acid content in corn steep liquor (source: J. Nielsen,
“Physiological Engineering Aspects of Penicillium Chrysogenum,”
Table 8.3, p. 243 (World Scientific 1997)).

| | Free | Total |
|---|---|---|
| Amino Acid | g/kg dry weight | g/kg dry weight |
| Alanine | 40.7 | 54.5 |
| Arginine | 2.4 | 20.3 |
| Aspartate | 2.2 | 19.9 |
| Cysteine | 0 | 1.3 |
| Glutamate | 7.7 | 40.2 |
| Glycine | 6.6 | 26.8 |
| Histidine | 0 | 31.8 |
| Isoleucine | 11.2 | 17.3 |
| Leucine | 35.5 | 39.3 |
| Lysine | 0 | 14.8 |
| Methionine | 6.5 | 6.9 |
| Phenylalanine | 26.2 | 27.4 |
| Proline | 27.7 | 48.2 |
| Serine | 10.7 | 19.0 |
| Threonine | 9.3 | 20.7 |
| Tyrosine | 1.3 | 6.5 |
| Valine | 20.1 | 30.5 |

In one embodiment, beneficial fermentation results can be achieved by adding corn steep powder in combination with yeast extract to the fermentation. Beneficial results with corn steep powder in combination with yeast extract can be achieved in the methods of the embodiments at corn steep powder usage levels of about 3 to about 20 g/L, about 5 to about 15 g/L, or about 8 to about 12 g/L and yeast extract usage levels of about 3 to 50 g/L, about 5 to about 30 g/L, or about 10 to about 30 g/L. The corn steep powder and yeast extract can also be fed throughout the course of the entire fermentation or a portion of the fermentation, continuously or delivered at intervals.

In other embodiments, the beneficial compounds from corn steep powder and/or yeast extract, such as glycine, histidine, isoleucine, proline, or phytate as well as combinations of these compounds can be added to the medium or broth to obtain a beneficial effect.

Various embodiments of the invention offer benefits relating to improving the titer and/or productivity of alcohol production by Clostridium phytofermentans by culturing the organism in a medium comprising one or more compounds comprising particular fatty acid moieties and/or culturing the organism under conditions of controlled pH.

Production of high levels of alcohol requires both the ability for the organism to thrive generally in the presence of elevated alcohol levels and the ability to continue to produce alcohol without undue inhibition or suppression by the alcohol and/or other components present. Frequently, different metabolic pathways will be implicated for each of these. For example, pathways related to cell growth generally include those related to protein production, membrane production as well as the production of all of the cellular subsystems necessary for the cell to survive. Pathways related to alcohol production will frequently be more specific, such as those pathways related to the metabolism of sugars leading to production of alcohol and the enzymes that are necessary for the production of alcohol and intermediates. The pathway for one alcohol, e.g., ethanol, can share some similar enzymes, etc., but will also have enzymes and substrates unique to that pathway. While there can be some overlap between these sets of pathways, it is not expected that enhancement of one will automatically result in the enhancement of the other.

In some cases, alcohol intolerance or alcohol-induced toxicity can be related to permeabilization of the cell membrane by elevated levels of alcohol, leading to leakage of intracellular enzymes and nutrients. In some other cases, alcohol tolerance and the ability to produce high alcohol titers is related to the ability of intracellular enzymes to withstand denaturing by the alcohol present, e.g., within the cell, whether due to production by the cell itself or from transport across the cell membrane. In some cases, a more robust membrane will allow a higher alcohol gradient to be present across the membrane, thus allowing the cells to grow and/or continue to produce alcohol at higher external alcohol concentrations. It has been demonstrated with Clostridium phytofermentans that in some fermentation processes an ethanol concentration attains a plateau of about 15 g/L after about 36-48 hours of batch fermentation, with carbon substrate remaining in the broth. In one embodiment lowering the fermentation pH to about 6.5 and/or adding unsaturated fatty acids resulted in a significant increase in the amount of ethanol produced by the organism, with about 35 g/L of ethanol observed in the broth following a 72-hour fermentation. In another embodiment it was observed that the productivity of the organism was higher (to about 0 g/L-d) when the ethanol titer was low and lower (to about 2 g/L-d) when the ethanol concentration was higher. Fermentation at reduced pH and/or with the addition of fatty acids resulted in about a five fold increase in the ethanol production rate.

In one embodiment, Q microbe is fermented with a substrate at about pH 5-8.5 In one embodiment a Q microbe is fermented at pH of about 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5.

### Fatty Acid Medium Component

In one aspect, the invention provides compositions for producing alcohol, e.g., ethanol, comprising a culture of Clostridium phytofermentans in a medium comprising a fatty acid comprising compound. The medium can also include a carbon source of biomass such as agricultural crops, crop residues, trees, wood chips, sawdust, paper, cardboard, or other materials containing cellulose, hemicellulosic, lignocellulose, pectin, polyglucose, polyfructose, and/or hydrolyzed forms of these (collectively, “Feedstock”). Additional nutrients can be present including sulfur- and nitrogen-containing compounds such as amino acids, proteins, hydrolyzed proteins, ammonia, urea, nitrate, nitrite, soy, soy derivatives, casein, casein derivatives, milk powder, milk derivatives, whey, yeast extract, hydrolyze yeast, autolyzed yeast, corn steep liquor, corn steep solids, monosodium glutamate, and/or other fermentation nitrogen sources, vitamins, cofactors and/or mineral supplements. The Feedstock can be pretreated or not, such as described in U.S. Provisional Patent Application No. 61/032,048, filed Feb. 27, 2008 or U.S. Provisional Application filed concurrently with this application on Mar. 9, 2009 as U.S. Provisional Patent Application No. 61/158,581, which are herein incorporated by reference in their entireties. The procedures and techniques for growing the organism to produce a fuel or other desirable chemical such as is described in incorporated Provisional U.S. Patent Application Nos. 61/032,048 or U.S. Provisional Application filed concurrently with this application on Mar. 9, 2009 as U.S. Provisional Patent Application No. 61/158,581, which are herein incorporated by reference in their entireties.

In one embodiment a fatty acid comprising compound of the composition can be a free fatty acid, fatty acid salt or soap, triacylglyceride, diacylglyceride, monoacylglyceride, phospholipid, lysophospholipid, fatty acid ester, or fatty acid amide. The fatty acid ester can comprise a long chain alcohol, short chain alcohol, medium chain alcohol, monohydrate alcohol, dihydric alcohol, trihydric alcohol, polyhydric alcohol, branched alcohol or other compound comprising a hydroxyl group. Preferred esters include those of methanol (fatty acid methyl esters), ethanol (fatty acid ethyl esters), n-propanol (fatty acid propyl esters) and isopropanol (fatty acid isopropyl esters), but other alcohols can be utilized as well such as those having 4 to 20 carbons. In some cases, longer chain alcohols and polyhydric alcohols can be used as well. Suitable longer chain or polyhydric alcohols include glycols (e.g. ethylene glycol, propylene glycol, etc.), glycerol, xylitol, mannitol, sorbitol, arabitol, or compounds such as polyethers containing one or more hydroxyl groups and polyethylene glycols. When more than one hydroxyl group is present, one or more of these groups can be bound to another chemical moiety (e.g. as an ester, an amide, an ether, etc.) or they can be free hydroxyl groups.

In another embodiment a fatty acid can comprise carbon chains of 8 to 40 carbons, and preferably 12 to 24 carbons. Particular embodiments can utilize a single fatty acid or a mixture of fatty acids. When a polyhydric alcohol is utilized, the fatty acid can be bound to only one hydroxyl group or to more than one hydroxyl group. In some embodiments, more than one fatty acid species can be bound to a single polyhydric alcohol. Examples of multiple fatty acids bound to a single polyhydric alcohol include fats and oils such as those derived from animals and vegetables, including corn, canola, safflower, rape seed, sunflower, soybean, olive, peanut, palm, palm kernel, fish, castor bean, tallow, lard, as well as partial glycerides and phospholipids.

While any C8-C30 fatty acid can be used, preferred fatty acids include unsaturated fatty acids, such as those with 1, 2, 3, or more carbon-carbon double bonds. Particularly preferred are those having an unsaturation at the omega-9 position (measured from the non-carboxyl end) or the delta-9 position (measured from the carboxyl end). An unsaturation at one or both of these positions can be accompanied by unsaturations at other positions as well. Also, while fatty acids with carbon chains of 8 to 30 carbons can be used preferred are those having carbon chains of 8 to 28 or 12 to 24, or 16 to 18 carbons. Examples of such fatty acids include oleic, stearic, palmitic, palmitoleic, linoleic, linolenic, lauric, myristic, arachidic, behenic, gadoleic, erucic, moroctic, or aractidonic acid. In some cases, a carbon-carbon double bond can be in a cis configuration, and in some cases a carbon-carbon double bond can be in a trans configuration. In some cases, more than one carbon-carbon double bond can be present. Some suitable fatty acids can have one or more cis and one or more trans carbon-carbon double bonds, such as with conjugated linoleic acid, and some other fatty acids, while some suitable fatty acids can have all carbon-carbon double bonds in a cis configuration or in a trans configuration.

In one embodiment a compound comprising one or more fatty acids (“fatty acids”) can be added to the medium early, intermediate, or late in a fermentation process of Clostridium phytofermentans. In one embodiment, the fatty acid compound can be added during one or more of the seed stages of the fermentation. In various embodiments, a fatty acid compound can be added prior to inoculation of the medium with Clostridium phytofermentans, or after inoculation, or simultaneous to inoculation. In another embodiment, the fatty acids can be added to a final fermentation medium, and can be added prior to inoculation, after inoculation, or simultaneous to inoculation of the medium with Clostridium phytofermentans. In some embodiments, the fatty acids can be added as several doses or continuously for at least a portion of the fermentation. Most preferably, the fatty acids can be added after alcohol, e.g., ethanol, begins to accumulate in the fermentation. In one embodiment, the fatty acids are added when the alcohol concentration reaches between about 2 g/L to 50 g/L. In another embodiment, the fatty acids are added when the alcohol concentration reaches between about 2 g/L to 10 g/L. In another embodiment, the fatty acids are added when the alcohol concentration reaches between about 5 g/L to 40 g/L. In another embodiment, the fatty acids are added when the alcohol concentration reaches between about 10 g/L to 30 g/L. In another embodiment, the fatty acids are added when the alcohol concentration reaches about 2 g/L. In another embodiment, the fatty acids can be added when the alcohol concentration reaches about 5 g/L. In another embodiment, the fatty acids can be added when the alcohol concentration reaches about 10 g/L. In another embodiment, the fatty acids can be added when the alcohol concentration reaches about 15 g/L. In another embodiment, the fatty acids can be added when the alcohol concentration reaches about 20 g/L. In another embodiment, the fatty acids can be added when the alcohol concentration reaches about 25 g/L. In another embodiment, the fatty acids can be added when the alcohol concentration reaches about 30 g/L. In another embodiment, the fatty acids can be added when the alcohol concentration reaches about 35 g/L. In another embodiment, the fatty acids can be added when the alcohol concentration reaches about 40 g/L. In another embodiment, the fatty acids can be added when the alcohol concentration reaches about 45 g/L. In another embodiment, the fatty acids can be added when the alcohol concentration reaches about 50 g/L. In some embodiments, the fatty acid can be added with one or more media components or near the beginning of the fermentation, as well as can be supplemented during fermentation. In one embodiment fatty acids are added when the alcohol concentration is 2 g/L, 3 g/L, 4 g/L, 5 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, or 50 g/L

In one embodiment, the fatty acids can be added as a solution in an alcohol; e.g., ethanol. In another embodiment, the fatty acids can be added as a colloid. In another embodiment, the fatty acids can be added with a surfactant.

While the amount of fatty acid compound to add can vary with the form of the fatty acid compound (for example a triacylglyceride or a phospholipid), and the specific fatty acid or combination of fatty acids being added (for example, oleic or palmitoleic acid), a suitable amount of fatty acid compound can be from about 1 g/L to about 3 g/L, reported as free fatty acid. In some embodiments, including runs of extended duration or those with extensive alcohol production or cellular growth, the fatty acid level can be maintained within the range of about 1 g/L to about 3 g/L or cycled through the range of about 1 g/L to about 3 g/L, reported as free fatty acid present in the supernatant are adsorbed to the surface of the cells or solid surfaces such as substrate or equipment. Suitable techniques for measuring the fatty acid level include separating at least a portion of the supernatant from the broth, with or without addition of a solvation aid, to assist desorption or solubilization of the fatty acid comprising compounds, and analyzing for fatty acid content with, for example a gas chromatograph. When the fermentation is operated as a fed batch, the fatty acid compound can be added all at once, or it can be added in portions or continuously, such as in relation to the medium components being fed to the fermenter.

In some embodiments, the rate that the fatty acid is taken up by the organism is modified by providing the fatty acid in a form that has only limited interaction with the organism, and then adding a compound that allows for increased interaction with the organism. A form that is present in a separate phase or a phase that cannot be consumed by the organism are examples of forms that have limited interaction with the organism. Compounds that increase the interaction are those that are able to hydrolyse the form of the fatty acid that is present, such as those with lipase activity, phospholipase activity, acids, bases, etc., or are able to solvate the fatty acids.

### Acidic Culture Conditions

In another aspect, the invention provides methods of producing alcohol; e.g., ethanol, comprising culturing Clostridium phytofermentans in a medium under conditions of controlled pH. In one embodiment, a culture of Clostridium phytofermentans can be grown at an acidic pH. The medium that the culture is grown in can include a carbon source such as agricultural crops, crop residues, trees, wood chips, sawdust, paper, cardboard, or other materials containing cellulose, hemicellulosic, lignocellulose, pectin, polyglucose, polyfructose, and/or hydrolyzed forms of these (collectively, “Feedstock”). Additional nutrients can be present including sulfur- and nitrogen-containing compounds such as amino acids, proteins, hydrolyzed proteins, ammonia, urea, nitrate, nitrite, soy, soy derivatives, casein, casein derivatives, milk powder, milk derivatives, whey, yeast extract, hydrolyze yeast, autolyzed yeast, corn steep liquor, corn steep solids, monosodium glutamate, and/or other fermentation nitrogen sources, vitamins, cofactors and/or mineral supplements. The Feedstock can be pretreated or not, such as described in U.S. Provisional Patent Application No. 61/032,048, filed Feb. 27, 2008 or U.S. Provisional Application No. 61/158,581, filed on Mar. 9, 2009, which are herein incorporated by reference in their entireties. The procedures and techniques for growing the organism to produce a fuel or other desirable chemical such as is described in incorporated Provisional U.S. Patent Application Nos. 61/032,048 or U.S. Provisional Application filed on Mar. 9, 2009, No. 61/158,581, which are herein incorporated by reference in their entireties.

In one embodiment, the pH of the medium is controlled at less than about pH 7.2 for at least a portion of the fermentation. In preferred embodiments, the pH is controlled within a range of about pH 3.0 to about 7.1 or about pH 4.5 to about 7.1, or about pH 5.0 to about 6.3, or about pH 5.5 to about 6.3, or about pH 6.0 to about 6.5, or about pH 5.5 to about 6.9 or about pH 6.2 to about 6.7. The pH can be controlled by the addition of a pH modifier. In the embodiments, a pH modifier can be an acid, a base, a buffer, or a material that reacts with other materials present to serve to raise of lower the pH. In some embodiments, more than one pH modifier can be used, such as more than one acid, more than one base, one or more acid with one or more bases, one or more acids with one or more buffers, one or more bases with one or more buffers, or one or more acids with one or more bases with one or more buffers. When more than one pH modifiers are utilized, they can be added at the same time or at different times. In some embodiments, one or more acids and one or more bases can be combined, resulting in a buffer. In some embodiments, media components, such as a carbon source or a nitrogen source can also serve as a pH modifier; suitable media components include those with high or low pH or those with buffering capacity. Exemplary media components include acid- or base-hydrolyzed plant polysaccharides having with residual acid or base, AFEX treated plant material with residual ammonia, lactic acid, corn steep solids or liquor.

In some embodiments, the pH modifier can be added as a part of the medium components prior to inoculation with the Clostridium phytofermentans. In other embodiments, the pH modifier can also be added after inoculation with the Clostridium phytofermentans. In some embodiments, sufficient buffer capacity can be added to the seed fermentation by way of various pH modifiers and/or other medium components and/or metabolites to provide adequate pH control during the final fermentation stage. In other cases, pH modifier can be added only to the final fermentation stage. In still other cases, pH modifier can be added to both the seed stage and the final stage. In one embodiment, the pH is monitored throughout the fermentation and is adjusted in response to changes in the fermentation. In one embodiment, the pH modifier is added whenever the pH of the fermentation changes by a pH value of about 0.005, 0.01, 0.05, 0.1, 0.2, 0.3, 0.4, 0.5 or more at any stage of the fermentation. In other embodiments, the pH modifier is added whenever the alcohol content of the fermentation is about 0.5 g/L, 1.0 g/L, 2.0 g/L, or 5.0 g/L or more. In some cases different types of pH modifiers can be utilized at different stages or points in the fermentation, such as a buffer being used at the seed stage, and base and/or acid added in the final fermenter, or an acid being used at one time and a base at another time.

In some embodiments, a constant pH can be utilized throughout the fermentation. In some embodiments, it can be advantageous to start the fermentation at one pH, and then to lower the pH during the course of the fermentation. In embodiments where the pH is lowered, the pH can be lowered in a stepwise fashion or a more gradual fashion. Suitable times for lowering the pH include during a lag phase of cellular growth, during an exponential phase of cellular growth, during a stationary phase of cellular growth, during a death phase of cellular growth, or before or during periods of cell proliferation. In some embodiments the pH can be lowered during more than one phase of growth. While in some embodiments, the pH can be lowered in a stepwise fashion, such as with the change occurring over a period of about 10 minutes or less, advantageous growth can be achieved in some embodiments by lowering the pH more gradually, such as over a period of about 10 minutes to about six hours or longer. In some embodiments, the timing and/or amount of pH reduction can be related to the growth conditions of the cells, such as in relation to the cell count, the alcohol produced, the alcohol present, or the rate of alcohol production. In some embodiments, the pH reduction can be made in relation to physical or chemical properties of the fermentation, such as viscosity, medium composition, gas production, off gas composition, etc.

Non-limiting examples of suitable buffers include salts of phosphoric acid, including monobasic, dibasic, and tribasic salts, mixtures of these salts and mixtures with the acid; salts of citric acid, including the various basic forms, mixtures and mixtures with the acid; and salts of carbonate.

Suitable acids and bases that can be used as pH modifiers include any liquid or gaseous acid or base that is compatible with the organism. Examples include ammonia, ammonium hydroxide, sulfuric acid, lactic acid, citric acid, phosphoric acid, sodium hydroxide, and HCl. In some cases, the selection of the acid or base can be influenced by the compatibility of the acid or base with equipment being used for fermentation. In some cases, both an acid addition, to lower pH or consume base, and a base addition, to raise pH or consume acid, can be used in the same fermentation.

The timing and amount of pH modifier to add can be determined from a measurement of the pH of the contents of the fermentor, such as by grab sample or by a submerged pH probe, or it can be determined based on other parameters such as the time into the fermentation, gas generation, viscosity, alcohol production, titration, etc. In some embodiments, a combination of these techniques can be used.

In one embodiment, the pH of the fermentation is initiated at a neutral pH and then is reduced to an acidic pH when the production of alcohol is detected. In another embodiment, the pH of the fermentation is initiated at an acidic pH and is maintained at an acidic pH until the fermentation reaches a stationary phase of growth.

### Fatty Acid Medium Component and Acidic Culture Conditions

In another embodiment, a combination of adding a fatty acid comprising compound to the medium and fermenting at reduced pH can be used. In some embodiments, addition of a fatty acid, such as a free fatty acid fulfills both techniques: adding a fatty acid compound and lowering the pH of the fermentation. In other embodiments, different compounds can be added to accomplish each technique. For example, a vegetable oil can be added to the medium to supply the fatty acid and then a mineral acid or an organic acid can be added during the fermentation to reduce the pH to a suitable level, as described above. When the fermentation includes both operation at reduced pH and addition of fatty acid comprising compounds, the methods and techniques described herein for each type of operation separately can be used together. In some embodiments, the operation at low pH and the presence of the fatty acid comprising compounds will be at the same time. In some embodiments, the presence of fatty acid comprising compounds will precede operation at low pH, and in some embodiments operation at low pH will precede the addition of fatty acid comprising compounds. In some embodiments, the operation at low pH and the presence of the fatty acid will be prior to inoculation with the Clostridium phytofermentans. In some embodiments, the operation at low pH will be prior to inoculation with the Clostridium phytofermentans and the presence of the fatty acid will occur after or during to inoculation with the Clostridium phytofermentans. In some embodiments, the presence of the fatty acid will be prior to inoculation with the Clostridium phytofermentans and the operation at low pH will occur after or during to inoculation with the Clostridium phytofermentans. In other embodiments, the operation at low pH and the presence of the fatty acid will be after inoculation with the Clostridium phytofermentans. In some embodiments, the operation at low pH and the presence of the fatty acid will be at other stages of fermentation.

Genetic Modification of Clostridium phytofermentans

In another aspect, the invention provides compositions and methods to produce a fuel such as one or more alcohols, e.g., ethanol, by the creation and use of a genetically modified Clostridium phytofermentans. This invention contemplates, in particular, regulating fermentative biochemical pathways, expression of saccharolytic enzymes, or increasing tolerance of environmental conditions during fermentation of Clostridium phytofermentans. In one embodiment, Clostridium phytofermentans is transformed with heterologous polynucleotides encoding one or more genes for the pathway, enzyme, or protein of interest. In another embodiment, Clostridium phytofermentans is transformed to produce multiple copies of one or more genes for the pathway, enzyme, or protein of interest. In one embodiment, Clostridium phytofermentans is transformed with heterologous polynucleotides encoding one or more genes encoding enzymes for the hydrolysis and/or fermentation of a hexose, wherein said genes are expressed at sufficient levels to confer upon said Clostridium phytofermentans transformant the ability to produce ethanol at increased concentrations, productivity levels or yields compared to Clostridium phytofermentans that is not transformed. In such ways, an enhanced rate of ethanol production can be achieved.

In another embodiment, the Clostridium phytofermentans is transformed with heterologous polynucleotides encoding one or more genes encoding saccharolytic enzymes for the saccharification of a polysaccharide, wherein said genes are expressed at sufficient levels to confer upon said Clostridium phytofermentans transformant the ability to saccharify a polysaccharide to mono-, di- or oligosaccharides at increased concentrations, rates of saccharification or yields of mono-, di- or oligosaccharides compared to Clostridium phytofermentans that is not transformed. The production of a saccharolytic enzyme by the host, and the subsequent release of that saccharolytic enzyme into the medium, reduces the amount of commercial enzyme necessary to degrade biomass or polysaccharides into fermentable monosaccharides and oligosaccharides. The saccharolytic DNA can be native to the host, although more often the DNA will be foreign, . . . , heterologous. Advantageous saccharolytic genes include cellulolytic, xylanolytic, and starch-degrading enzymes such as cellulases, xylanases, and amylases. The saccharolytic enzymes can be at least partially secreted by the host, or it can be accumulated substantially intracellularly for subsequent release. Advantageously, intracellularly-accumulated enzymes which are thermostable, can be released when desired by heat-induced lysis. Combinations of enzymes can be encoded by the heterologous DNA, some of which are secreted, and some of which are accumulated.

Other modifications can be made to enhance the ethanol production of the recombinant bacteria of the subject invention. For example, the host can further comprise an additional heterologous DNA segment, the expression product of which is a protein involved in the transport of mono- and/or oligosaccharides into the recombinant host. Likewise, additional genes from the glycolytic pathway can be incorporated into the host. In such ways, an enhanced rate of ethanol production can be achieved.

In order to improve the production of biofuels (e.g. ethanol), modifications can be made in transcriptional regulators, genes for the formation of organic acids, carbohydrate transporter genes, sporulation genes, genes that influence the formation/regenerate of enzymatic cofactors, genes that influence ethanol tolerance, genes that influence salt tolerance, genes that influence growth rate, genes that influence oxygen tolerance, genes that influence catabolite repression, genes that influence hydrogen production, genes that influence resistance to heavy metals, genes that influence resistance to acids or genes that influence resistance to aldehydes.

Those skilled in the art will appreciate that a number of modifications can be made to the methods exemplified herein. For example, a variety of promoters can be utilized to drive expression of the heterologous genes in the recombinant Clostridium phytofermentans host. The skilled artisan, having the benefit of the instant disclosure, will be able to readily choose and utilize any one of the various promoters available for this purpose. Similarly, skilled artisans, as a matter of routine preference, can utilize a higher copy number plasmid. In another embodiment, constructs can be prepared for chromosomal integration of the desired genes. Chromosomal integration of foreign genes can offer several advantages over plasmid-based constructions, the latter having certain limitations for commercial processes. Ethanologenic genes have been integrated chromosomally in E. coli B; see Ohta et al. (1991) Appl. Environ. Microbiol. 57:893-900. In general, this is accomplished by purification of a DNA fragment containing (1) the desired genes upstream from an antibiotic resistance gene and (2) a fragment of homologous DNA from the target organism. This DNA can be ligated to form circles without replicons and used for transformation. Thus, the gene of interest can be introduced in a heterologous host such as E. coli, and short, random fragments can be isolated and ligated in Clostridium phytofermentans to promote homologous recombination.

### Biofuel Plant and Process of Producing Biofuel:

Large Scale Ethanol Production from Biomass

Generally, there are two basic approaches to producing fuel grade ethanol from biomass on a large scale utilizing of microbial cells, especially C. phytofermentans cells. In the first method, one first hydrolyzes a biomass material that includes high molecular weight carbohydrates to lower molecular weight carbohydrates, and then ferments the lower molecular weight carbohydrates utilizing of microbial cells to produce ethanol. In the second method, one ferments the biomass material itself without chemical and/or enzymatic pretreatment. In the first method, hydrolysis can be accomplished using acids, e.g., Bronsted acids (e.g., sulfuric or hydrochloric acid), bases, e.g., sodium hydroxide, hydrothermal processes, ammonia fiber explosion processes (“AFEX”), lime processes, enzymes, or combination of these. Hydrogen, and other products of the fermentation can be captured and purified if desired, or disposed of, e.g., by burning. For example, the hydrogen gas can be flared, or used as an energy source in the process, e.g., to drive a steam boiler, e.g., by burning. Hydrolysis and/or steam treatment of the biomass can, e.g., increase porosity and/or surface area of the biomass, often leaving the cellulosic materials more exposed to the microbial cells, which can increase fermentation rate and yield. Removal of lignin can, e.g., provide a combustible fuel for driving a boiler, and can also, e.g., increase porosity and/or surface area of the biomass, often increasing fermentation rate and yield. Generally, in any of the below described embodiments, the initial concentration of the carbohydrates in the medium is greater than 20 mM, e.g., greater than 30 mM, 50 mM, 75 mM, 100 mM, 150 mM, 200 mM, or even greater than 500 mM.

Biomass Processing Plant and Process of Producing Products from Biomass

In one aspect, the invention features a fuel plant that includes a hydrolysis unit configured to hydrolyze a biomass material that includes a high molecular weight carbohydrate, a fermentor configured to house a medium with Clostridium phytofermentans cells or another C5/C6 hydrolyzing organism dispersed therein, and one or more product recovery system(s) to isolate a product or products and associated by-products and co-products.

In another aspect, the invention features methods of making a product or products that include combining Clostridium phytofermentans cells or another C5/C6 hydrolyzing organism and a biomass feed in a medium, and fermenting the biomass material under conditions and for a time sufficient to produce a biofuel, chemical product or fermentive end-products, e.g. ethanol, propanol, hydrogen, lignin, terpenoids, and the like as described in paragraph 0063.

In another aspect, the invention features products made by any of the processes described herein.

### Large Scale Chemical Production From Biomass

Generally, there are two basic approaches to producing chemical products from biomass on a large scale utilizing microorganisms such as Clostridium phytofermentans or other C5/C6 hydrolyzing organisms. In all methods, depending on the type of biomass and its physical manifestation, one of the processes can comprise a milling of the carbonaceous material, via wet or dry milling, to reduce the material in size and increase the surface to volume ratio (physical modification).

In a first method, one first hydrolyzes a biomass material that includes high molecular weight carbohydrates to delignify it or to separate the carbohydrate compounds from noncarbohydrate compounds. Using any combination of heat, chemical, and/or enzymatic treatment, the hydrolyzed material can be separated to form liquid and dewatered streams, which may or may not be separately treated and kept separate or recombined, and then ferments the lower molecular weight carbohydrates utilizing Clostridium phytofermentans cells or another C5/C6 hydrolyzing organism to produce one or more chemical products. In the second method, one ferments the biomass material itself without heat, chemical, and/or enzymatic pretreatment. In the first method, hydrolysis can be accomplished using acids (e.g. sulfuric or hydrochloric acids), bases (e.g. sodium hydroxide), hydrothermal processes, ammonia fiber explosion processes (“AFEX”), lime processes, enzymes, or combination of these. Hydrolysis and/or steam treatment of the biomass can, e.g., increase porosity and/or surface area of the biomass, often leaving the cellulosic materials more exposed to any C5/C6 hydrolyzing organism, such as C. phytofermentans, which can increase fermentation rate and yield. Hydrolysis and/or steam treatment of the biomass can, e.g., produce by-products or co-products which can be separated or treated to improve fermentation rate and yield, or used to produce power to run the process, or used as products with or without further processing. Removal of lignin can, e.g., provide a combustible fuel for driving a boiler. Gaseous, e.g., hydrogen and CO₂, liquid, e.g. ethanol and organic acids, and solid, e.g. lignin, products of the fermentation can be captured and purified if desired, or disposed of, e.g., by burning. For example, the hydrogen gas can be flared, or used as an energy source in the process, e.g., to drive a steam boiler, e.g., by burning. Products exiting the fermentor can be further processed, e.g. ethanol may be transferred to distillation and rectification, producing a concentrated ethanol mixture or solids may be separated for use to provide energy or as chemical products. It is understood that other methods of producing fermentive end products or biofuels can incorporate any and all of the processes described as well as additional or substitute processes that may be developed to economically or mechanically streamline these methods, all of which are meant to be incorporated in their entirety within the scope of this invention.

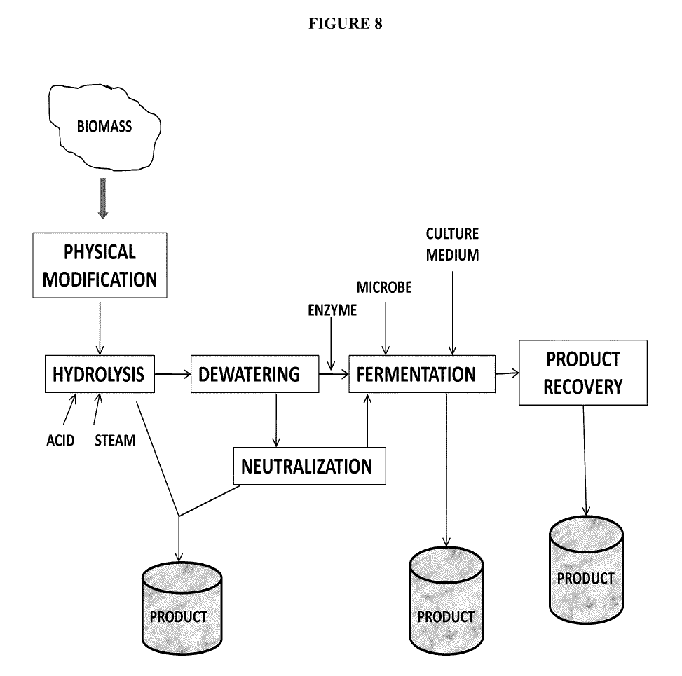

FIG. 8 is an example of a method for producing chemical products from biomass by first treating biomass with an acid at elevated temperature and pressure in a hydrolysis unit. The biomass may first be heated by addition of hot water or steam. The biomass may be acidified by bubbling gaseous sulfur dioxide through the biomass that is suspended in water, or by adding a strong acid, e.g., sulfuric, hydrochloric, or nitric acid with or without preheating/presteaming/water addition. During the acidification, the pH is maintained at a low level, e.g., below about 5. The temperature and pressure may be elevated after acid addition. In addition to the acid already in the acidification unit, optionally, a metal salt such as ferrous sulfate, ferric sulfate, ferric chloride, aluminum sulfate, aluminum chloride, magnesium sulfate, or mixtures of these can be added to aid in the hydrolysis of the biomass. The acid-impregnated biomass is fed into the hydrolysis section of the pretreatment unit. Steam is injected into the hydrolysis portion of the pretreatment unit to directly contact and heat the biomass to the desired temperature. The temperature of the biomass after steam addition is, e.g., between about 130° C. and 220° C. The hydrolysate is then discharged into the flash tank portion of the pretreatment unit, and is held in the tank for a period of time to further hydrolyze the biomass, e.g., into oligosaccharides and monomeric sugars. Steam explosion may also be used to further break down biomass. Alternatively, the biomass can be subject to discharge through a pressure lock for any high-pressure pretreatment process. Hydrolysate is then discharged from the pretreatment reactor, with or without the addition of water, e.g., at solids concentrations between about 15% and 60%.

After pretreatment, the biomass may be dewatered and/or washed with a quantity of water, e.g. by squeezing or by centrifugation, or by filtration using, e.g. a countercurrent extractor, wash press, filter press, pressure filter, a screw conveyor extractor, or a vacuum belt extractor to remove acidified fluid. The acidified fluid, with or without further treatment, e.g. addition of alkali (e.g. lime) and or ammonia (e.g. ammonium phosphate), can be re-used, e.g., in the acidification portion of the pretreatment unit, or added to the fermentation, or collected for other use/treatment. Products may be derived from treatment of the acidified fluid, e.g., gypsum or ammonium phosphate. Enzymes or a mixture of enzymes can be added during pretreatment to assist, e.g. endoglucanases, exoglucanases, cellobiohydrolases (CBH), beta-glucosidases, glycoside hydrolases, glycosyltransferases, lyases, and esterases active against components of cellulose, hemicelluloses, pectin, and starch, in the hydrolysis of high molecular weight components.

The fermentor is fed with hydrolyzed biomass, any liquid fraction from biomass pretreatment, an active seed culture of Clostridium phytofermentans cells, if desired a co-fermenting microbe, e.g., yeast or E. coli, and, if required, nutrients to promote growth of Clostridium phytofermentans or other microbes. Alternatively, the pretreated biomass or liquid fraction can be split into multiple fermentors, each containing a different strain of Clostridium phytofermentans and/or other microbes, and each operating under specific physical conditions. Fermentation is allowed to proceed for a period of time, e.g., between about 15 and 150 hours, while maintaining a temperature of, e.g., between about 25° C. and 50° C. Gas produced during the fermentation is swept from fermentor and is discharged, collected, or flared with or without additional processing, e.g. hydrogen gas may be collected and used as a power source or purified as a co-product.

After fermentation, the contents of the fermentor are transferred to product recovery. Products are extracted, e.g., ethanol is recovered through distilled and rectification.

### Chemical Production From Biomass Without Pretreatment

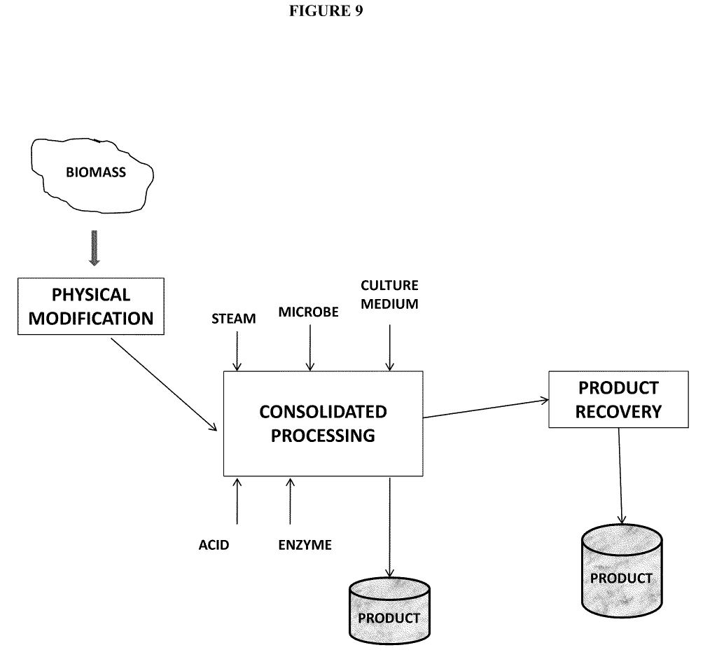

FIG. 9 depicts a method for producing chemicals from biomass by charging biomass to a fermentation vessel. The biomass may be allowed to soak for a period of time, with or without addition of heat, water, enzymes, or acid/alkali. The pressure in the processing vessel may be maintained at or above atmospheric pressure. Acid or alkali may be added at the end of the pretreatment period for neutralization. At the end of the pretreatment period, or at the same time as pretreatment begins, an active seed culture of Clostridium phytofermentans cells or another C5/C6 hydrolyzing organism and, if desired, a co-fermenting microbe, e.g., yeast or E. coli, and, if required, nutrients to promote growth of Clostridium phytofermentans or other microbes are added. Fermentation is allowed to proceed as described above. After fermentation, the contents of the fermentor are transferred to product recovery as described above.

Any combination of the chemical production methods and/or features can be utilized to make a hybrid production method. In any of the methods described herein, products may be removed, added, or combined at any step. Clostridium phytofermentans can be used alone, or synergistically in combination with one or more other microbes (e.g. yeasts, fungi, or other bacteria). Different methods may be used within a single plant to produce different products.

In another aspect, the invention features a fuel plant that includes a hydrolysis unit configured to hydrolyze a biomass material that includes a high molecular weight carbohydrate, and a fermentor configured to house a medium and contains Clostridium phytofermentans cells dispersed therein.

In another aspect, the invention features methods of making a fuel or fuels that include combining Clostridium phytofermentans cells and a lignocellulosic material (and/or other biomass material) in a medium, and fermenting the lignocellulosic material under conditions and for a time sufficient to produce a fuel or fuels, e.g., ethanol, propanol and/or hydrogen or another chemical compound.

In some embodiments, the present invention provides a process for producing ethanol and hydrogen from biomass using acid hydrolysis pretreatment. In some embodiments, the present invention provides a process for producing ethanol and hydrogen from biomass using enzymatic hydrolysis pretreatment. Other embodiments provide a process for producing ethanol and hydrogen from biomass using biomass that has not been enzymatically pretreated. Still other embodiments disclose a process for producing ethanol and hydrogen from biomass using biomass that has not been chemically or enzymatically pretreated, but is optionally steam treated.

In another aspect, the invention features products made by any of the processes described herein.

### EXAMPLES

The following examples serve to illustrate certain preferred embodiments and aspects and are not to be construed as limiting the scope thereof.

### Example 1

### Comparison of Batch and Fed Batch Fermentation of the Q Microbe—Feeding Medium Components Only

Experimental Conditions:

Three stirred tank reactors (STRs), or fermentors, were operated under fed-batch mode to study cellobiose fermentation using Q-microbes. A fourth STR was operated as a control under batch mode. All STRs whether operated under fed batch or batch mode contained 30 g/L cellobiose substrate at time zero. All reagents were obtained from Sigma-Aldrich, St. Louis, Mo., and were reagent grade or better.

Inoculum Preparation:

Frozen culture (stored at −80° C.) was used to create an inoculum that was propagated anaerobically at 35° C. for 48 hours in 10 mL tubes containing 0.3% cellobiose along with 4 g/L KH₂PO₄, 8 g/L K₂HPO₄, 1 g/L (NH₄)₂SO₄, 0.6 g/L cysteine-HCl, 6 g/L Ambrex 695 yeast extract (Sensient, Juneau, Wis.) in DI water (liquid volume about 10 ml). Thereafter, the inoculum was grown at 35° C. for 48 hours in 100 mL serum using 2% (v/v) seed size. The serum vials contained 20 g/L cellobiose, 1.5 g/L KH₂PO₄, 2.9 g/L K₂HPO₄, 2.1 g/L urea, 2 g/L cysteine-HCl, 10 g/L MOPS buffer, 3 g/L sodium citrate, 1 g/L MgCl₂.6H₂O, 0.15 g/L CaCl₂.2H₂O, 0.00125 g/L FeSO₄.7H₂O in DI water. Aliquots of grown inocula were examined under a microscope for microbial contamination and centrifuged at 3000 rpm for 15 minutes to concentrate the biomass (to about 2-4 g/L total suspended solids) for inoculation of the fermentor. The same inoculums preparation procedure was used for both batch as well as fed-batch fermentations.

Batch Fermentation (Control):

Medium was prepared containing 50 g/L cellobiose, 1.5 g/L KH₂PO₄, 2.9 g/L K₂HPO₄, 2.1 g/L urea, 2 g/L cysteine-HCl, 10 g/L MOPS buffer, 3 g/L sodium citrate, 1 g/L MgCl₂.6H₂O, 0.15 g/L CaCl₂.2H₂O, 0.00125 g/L FeSO₄.7H₂O in DI water. The pH of the medium was adjusted to 7.5 with 2 N NaOH, and 300 ml of the medium was transferred to each 500 mL fermentor. After degassing the vessel (600 mbar vacuum for at least 5 minutes with the medium at about room temperature, followed by nitrogen purge of the headspace to raise the vessel pressure back to atmospheric), the vessel was sterilized by autoclaving at 121° C. temperature and 15 psi for 30 minutes. Once the autoclaved vessel was cooled to room temperature, it was inoculated with 10% (v/v) inoculum (concentrated seed volume/final fermentation volume) using a 60 mL sterile syringe. The broth was cultured for 151 hours at 35° C., agitation at 125 rpm.

The fermentor was sampled each day, and analyzed for cellobiose, lactic acid, formic acid, acetic acid, and ethanol using HPLC equipped with Aminex® HPX-87H Exclusion column (300 mm×7.8 mm) and RI detector. 0.005 N H₂SO₄ was used as the mobile phase at 0.6 mL/minute, and the column was maintained at 55° C.

Fed-batch Fermentation:

Medium was prepared containing 30 g/l cellobiose, 1.5 g/L KH₂PO₄, 2.9 g/L K₂HPO₄, 2.1 g/L urea, 2 g/L cysteine-HCl, 10 g/L MOPS buffer, 3 g/L sodium citrate, 1 g/L MgCl₂.6H₂O, 0.15 g/L CaCl₂.2H₂O, 0.00125 g/L FeSO₄.7H₂O in DI water. The pH of the media was adjusted to 7.5 with 2 N NaOH. Medium (300 mL) was added to each of three 500 mL fermentation vessels. The fermentors were degassed in the same manner as the batch fermentation, followed by autoclaving at 121° C. and 15 psi for 30 minutes. Once the autoclaved vessels were cooled to the room temperature, they were inoculated with 10% (v/v) inoculums (concentrated seed volume/final fermentation volume) using a 60 mL sterile syringe. The broth was cultured for 184 hours at 35° C., agitation at 125 rpm. The broth was supplemented with 25 mL of fresh medium with 250 g/L cellobiose along with 1.5 g/L KH₂PO₄, 2.9 g/L K₂HPO₄, 2.1 g/L urea, 2 g/L cysteine-HCl, 10 g/L MOPS buffer, 3 g/L sodium citrate, 1 g/L MgCl₂.6H₂O, 0.15 g/L CaCl₂.2H₂O, 0.00125 g/L FeSO₄.7H₂O in DI water were added to the fermentors at 24, 48, 72, 96, 120, 144, and 168 hours after inoculation of the fermentor. The supplemental medium had been sterilized.

Fermentor Monitoring

The fermentors were sampled every day, and analyzed for cellobiose, lactic acid, formic acid, acetic acid, and ethanol using an HPLC equipped with Aminex® HPX-87H Exclusion column (300 mm×7.8 mm) (Bio-Rad, Hercules, Calif.) and RI detector. 0.005 N H₂SO₄ was used as the mobile phase at 0.6 mL/minute, and the column was maintained at 55° C.

Results:

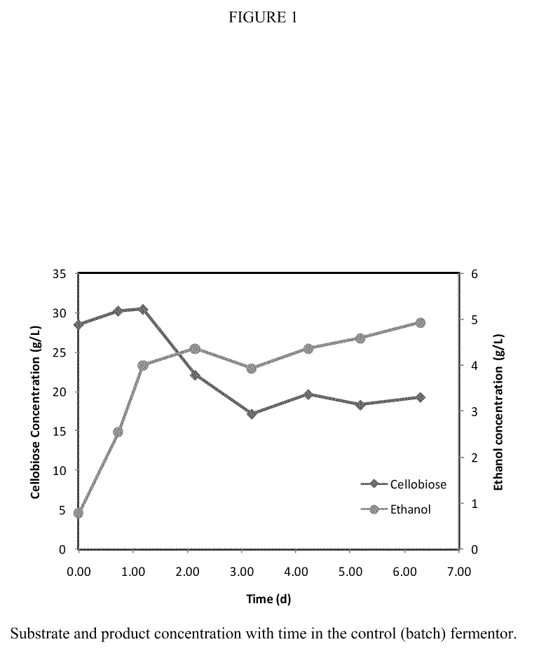

FIG. 1 shows the substrate (cellobiose) and product (ethanol) concentration throughout the fermentation run for the control fermentor, which was operated under batch mode. It is evident from the figure that ethanol concentration in the broth reached a plateau after about 30 hours. Although the control fermentor was kept running for over six days, there was no considerable increase in the ethanol concentration.

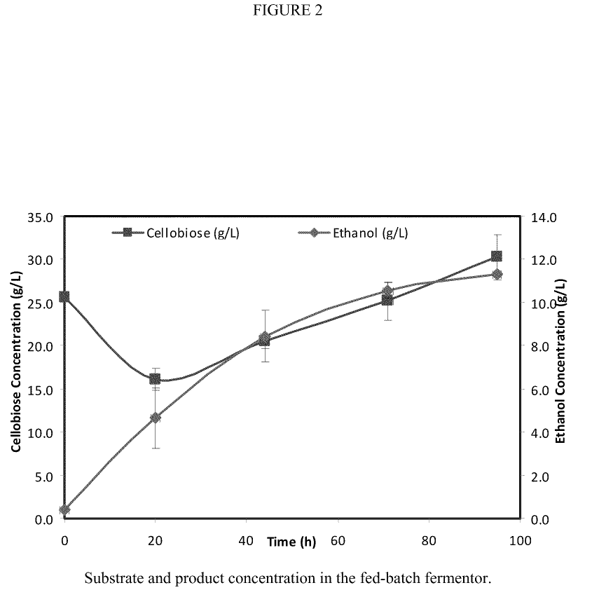

FIG. 2 shows the substrate (cellobiose) and product (ethanol) profile for the fermentors operated under fed-batch mode. Values shown are the average of the three fermentations. As shown in the figure the concentration of ethanol continued to increase with feeding of fresh nutrients and substrate. The maximum ethanol concentration achieved through fed-batch operation was about 12 g/L, which is more than double the titer achieved in the control fermentor operated batchwise.

In addition to the higher ethanol titer, the fed batch process (carbon substrate concentration at about 20-30 g/L) also resulted in higher productivity and in lower production of acids on both a g/g of sugar fermented basis and a g/g of ethanol produced basis, as shown in Table 7. It is also significant that the particular media and fermentation conditions used resulted in higher early productivity (approximately 4 g/L-day during early part of the fermentation) than has been reported for this organism.

TABLE 7
Comparison of important fermentation parameters for
batch and fed-batch experiments.

| Parameters | Batch | Fed-batch |
|---|---|---|
| Sugar loaded, g | 9.00 | 38.75 |
| Sugar fermented, g | 3.22 | 19.63 |
| Ethanol concentration, g/L | 4.93 | 12.29 |
| Ethanol yield, g/g sugar fermented | 0.46 | 0.27 |
| Acids yield, g/g sugar fermented | 0.19 | 0.02 |
| Ethanol productivity, g/L-d | 0.78 | 1.83 |

### Example 2

### Fed Batch Operation with Insoluble Carbon Source

Batch and fed batch fermentations is performed using an insoluble carbon source, such as microcrystalline cellulose. The fermentation media is made up as in Example 1, except that microcrystalline cellulose is substituted for cellobiose in the final production medium. (Microcrystalline cellulose is substituted for cellobiose in one or more of the other fermentation or seed stages instead of or in addition to the final fermentation medium.) The results for using microcrystalline cellulose trend-wise is similar to using cellobiose, with higher yield and productivity of ethanol in fed batch when compared to the batch operation. Similarly, higher conversion of sugar to ethanol (g ethanol/g of sugar fermented) and lower conversion of sugar to acids (g acid/g sugar fermented and g acid/g ethanol) occurs in the fed batch operation when compared to the batch operation. Similar results, trend wise, are achieved with more complicated insoluble carbon sources such as ground wood, ground plant matter, or pretreated ground wood or pretreated ground plant matter and with cellulosic, lignocellulosic, or hemicellulosic materials or waste streams. However, the absolute rates of production of ethanol or other targeted product varies either higher or lower than the cellobiose results due at least in part to the presence of additional nutrients or inhibiting agents in the more complex substrate

### Example 3

### Fed Batch Operation with Cell Augmentation

A fed batch fermentation is performed with the addition of fresh cells to the broth during the course of the fermentation. A fermentation medium is prepared and inoculated as in Example 1. At 24-hour intervals, fresh inoculum (2-3% v/v) is added to the fermentation and samples of the broth are analyzed as in Example 1. After about 2-4 days, the broth is harvested. At harvest, the ethanol content of the broth is greater than about 6 g/l, demonstrating a substantial increase over the batch operation, also demonstrating the increase in productivity.

Similar results can be seen with the insoluble and more complex carbon source-based media of Example 2. Augmentation of the fermentation broth with fresh cells is also used in situations where higher concentrations of carbon substrate are present, such as up to about 100 g/L or, in some cases, higher.

### Example 4

### Fed Batch Operation with Combined Cell Augmentation and Medium Addition

A fed batch fermentation is also performed with the addition of fresh cells and fresh medium components to the broth during the course of the fermentation. A fermentation medium can be prepared and inoculated as described in Example 1. At 24-hour intervals, fresh inoculum (2-3% v/v) is added to the fermentation as well as the medium as in Example 1. Samples of the broth are analyzed as in Example 1. After about 2-4 days, the broth is harvested. At harvest, the ethanol yield and productivity is higher than for the fed batch fermentation without cell augmentation. Similarly, improved carbon utilization (g ethanol/g sugar fermented) and reduced acid production (g acid/g ethanol and g acid/g sugar fermented) as compared to the fed batch without cell augmentation is demonstrated.

Similar results are seen with the insoluble and more complex carbon source-based media of Example 2.

### Example 5

### Fed-Batch Fermentation with Yeast Extract Present

Four stirred tank reactors (STR), each having 300 mL media containing 25 g/L cellobiose, 1.5 g/L KH₂PO₄, 2.9 g/L K₂HPO₄, 4.6 g/L ammonium sulfate, 2 g/L cysteine-HCl, 3 g/L sodium citrate, 1 g/L MgCl₂.6H₂O, 0.15 g/L CaCl₂.2H₂O, 0.00125 g/L FeSO₄.7H₂O, and levels of yeast extract (Bacto,™ Becton Dickinson, Franklin Lakes, N.J.) (10, 15, 20 and 30 g/L) were used. Analysis of Bacto yeast extract is provided in Table 8. All STRs were incubated at 35° C., 125 rpm and operated as fed-batch, with additional cellobiose added (25 ml of 200 g/l solution) every 24 hr. Ethanol production was monitored throughout the course of the fermentation. Table 9 shows the ethanol concentrations from these experiments.

TABLE 8
Typical Composition of Bacto Yeast Extract
(source: Bacto datasheet, Becton Dickinson).

| | |
|---|---|
| Total nitrogen | 10.9% |
| amino nitrogen | 6.0% |
| ash | 11.2% |
| loss on drying | 3.1% |

| Amino Acid Analysis | Free (%) | Total (%) |
|---|---|---|
| Alanine | 4.4 | 5.6 |
| Aspartic acid | 1.6 | 5.3 |
| Histidine | 0.6 | 1.3 |
| Leucine | 3.0 | 4.1 |
| Methionine | 0.6 | 0.8 |
| Proline | 0.8 | 2.0 |
| Threonine | 1.1 | 1.6 |
| Tyrosine | 0.8 | 1.2 |
| Arginine | 1.4 | 2.6 |
| Cystine | 0.2 | (destroyed by hydrolysis) |
| Glycine | 1.0 | 3.0 |
| Isoleucine | 1.8 | 3.0 |
| Lysine | 1.9 | 4.6 |
| Phenylalanine | 2.0 | 2.6 |
| Serine | 2.6 | 1.6 |
| Tryptophan | 0.5 | (destroyed by hydrolysis) |
| Valine | 2.2 | 3.5 |

TABLE 9
Ethanol Concentration in g/L at Different Times and
for Each Medium Formulation.

| Time, hrs | 10 g/L YE | 15 g/L YE | 20 g/L YE | 30 g/L YE |
|---|---|---|---|---|
| 0 | 0.1234 | 0.1651 | 0.1353 | 0.1389 |
| 18 | 5.1174 | 6.8853 | 6.3372 | 8.1321 |
| 45 | 7.6586 | 9.2264 | 9.0582 | 9.438 |
| 76 | 9.7681 | 11.654 | 11.6886 | 11.4085 |
| 100 | 11.2567 | 13.0663 | 13.4312 | 12.756 |
| 124 | 11.485 | 11.9113 | 11.9634 | 12.1095 |
| 148 | 11.8731 | 12.4778 | 11.865 | 12.0946 |

The volumetric productivity at 18 hours for the different media compositions was 2.00, 2.69, 2.48, 3.20 g/L-day for the 10, 15, 20, and 30 g/L yeast extract media, respectively.

These results show an increase in ethanol titer and overall productivity with increasing amounts of yeast extract and demonstrate production of ethanol up to about 15 g/L, and instantaneous productivity of greater than about 10 g/L-day.

### Example 6

### Ethanol Production by C. Phytofermentans with Different Vegetable Oil Supplements

The effect of fatty acid supplementation during fermentation on ethanol production was evaluated by growing cultures of Clostridium phytofermentans on cellobiose medium under agitation until the production of ethanol stopped. Fresh medium comprising of 10 mL of freshly grown inoculum was combined with 2 g/L of a vegetable oil. The ethanol production was monitored for an additional 100 hours.

### Reagents Used:

All chemicals except the vegetable oils, were at least reagent grade from Sigma-Aldrich (St. Louis, Mo.). The vegetable oils were Great Value brand oils, marketed by Wal-Mart (Bentonville, Ark.).

### Degassing and Sterilization Procedure:

All reactors and serum vials used for inoculum propagation were degassed under vacuum under a nitrogen purge. A minimum of three degassing cycles were performed. The vessel was sterilized by autoclaving at 121° C. temperature and 15 PSI pressure for 30 minutes.

### Inoculum Preparation:

Frozen culture (stored at −80° C.) was propagated at 35° C. for 48 hours in 10 mL tubes containing 0.3% cellobiose along with 1.5 g/L KH₂PO₄, 2.9 g/L K₂HPO₄, 4.6 g/L ammonium sulfate, 2 g/L cysteine-HCl, 1 g/L MgCl₂ 6H₂O, 0.15 g/L CaCl₂ 2H₂O, 0.00125 g/L FeSO₄ 7H₂O in DI water. The pH of the media was adjusted to 7.5 with 2 N NaOH. After autoclaving, the inoculums were grown at 35° C. for 24 hours in 100 mL serum using 2% (v/v) seed size. The serum vials contained 20 g/L cellobiose, 1.5 g/L KH₂PO₄, 2.9 g/L K₂HPO₄, 4.6 g/L ammonium sulfate, 2 g/L cysteine-HCl, 3 g/L sodium citrate, 1 g/L MgCl₂ 6H₂O, 0.15 g/L CaCl₂ 2H₂O, 0.00125 g/L FeSO₄ 7H₂O in DI water. Inoculums were centrifuged at 3000 rpm for 15 minutes to concentrate the cells (2-4 g/L total suspended solids) prior to use as inoculum for the fermentors.

Final Fermentation—Screening Experiment with Different Oils:

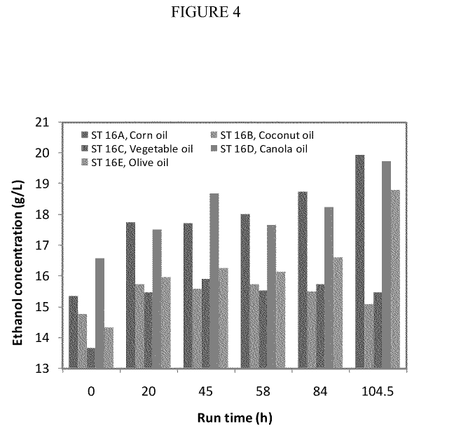

Five stirred tank reactors were filled with 50 mL media containing 20 g/L cellobiose, 1.5 g/L KH₂PO₄, 2.9 g/L K₂HPO₄, 4.6 g/L ammonium sulfate, 2 g/L cysteine-HCl, 3 g/L sodium citrate, 1 g/L MgCl₂.6H₂O, 0.15 g/L CaCl₂ 2H₂O, 0.00125 g/L FeSO₄.7H₂O, 6 g/L yeast extract (Bacto). Each reactor was inoculated with concentrated cells from one serum vial. The fermentors were operated under batch mode until ethanol production stopped. The ethanol concentration of each reactor is shown in Table 10. Residual cellobiose in the media at this point was about 15-20 g/L. Each reactor was then supplemented with about 10 mL of freshly grown inoculum and 2 g/L of a vegetable oil as shown in Table 10 Fermentation was continued for another 100 hours. Final ethanol concentrations are shown in Table 10. Ethanol concentrations throughout the period after supplementation are shown in FIG. 4 and Table 11.

TABLE 10
Ethanol Concentration of the Different Reactors Prior to
Medium Supplementation.

| | Reactor | | | | |
|---|---|---|---|---|---|
| | 1 | 2 | 3 | 4 | 5 |
| Ethanol | Corn | Coconut | Soybean | Canola | Olive |
| Concentration | 15.4 | 14.8 | 13.7 | 16.6 | 14.4 |
| Prior to Medium | | | | | |
| Supplementation | | | | | |
| Oil added | 2 g/L | 2 g/L | 2 g/L | 2 g/L | 2 g/L |
| Final Ethanol | 20.0 | 15.1 | 15.5 | 19.8 | 18.8 |
| Concentration | | | | | |

TABLE 11
Ethanol Concentration v. Time.
Ethanol concentration (g/L)

| | | | | ST | |
|---|---|---|---|---|---|
| Run | ST 16A, | ST 16B, | ST 16C, | 16D, | ST 16E, |
| time | Corn | Coconut | Soybean | Canola | Olive |
| (hr) | oil | oil | oil | oil | oil |
| 0 | 15.4 | 14.8 | 13.7 | 16.6 | 14.4 |
| 20 | 17.8 | 15.7 | 15.5 | 17.5 | 16.0 |
| 45 | 17.7 | 15.6 | 15.9 | 18.7 | 16.3 |
| 58 | 18.0 | 15.7 | 15.5 | 17.7 | 16.2 |
| 84 | 18.8 | 15.5 | 15.7 | 18.3 | 16.6 |
| 104.5 | 20.0 | 15.1 | 15.5 | 19.8 | 18.8 |

### Results

Addition of corn, soybean, canola, coconut oil and olive oil to the fermentations all resulted in further production of ethanol. In addition, the greatest increase in ethanol resulted from supplementation with oils high in oleic acid (olive, canola, soy bean and corn oil, as shown in Table 14), with the linoleic acid content also contributing to an increase in yield.

### Example 7

### Ethanol Production by Clostridium phytofermentans at Reduced pH

Bioreactors contained 300 mL media containing 20 g/L cellobiose, 1.5 g/L KH₂PO₄, 2.9 g/L K₂HPO₄, 4.6 g/L ammonium sulfate, 2 g/L cysteine-HCl, 3 g/L sodium citrate, 1 g/L MgCl₂.6H₂O, 0.15 g/L CaCl₂ 2H₂O, 0.00125 g/L FeSO₄.7H₂O, 6 g/L yeast extract (Bacto). The fermentors were operated under fed-batch mode by continuously feeding concentrated media containing 200 g/L cellobiose at 1.4 mL/h. The bioreactors were operated at controlled pH of 7.5, 7 and 6.5, respectively.

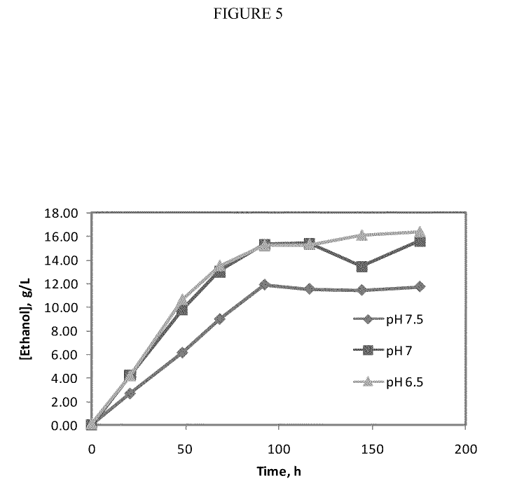

The fermenters were monitored for ethanol concentration throughout the fermentation. The results are shown in Table 12 and FIG. 5. The results show that fermentation at pH less than 7.5 results in an increase in the concentration of ethanol and an increase in the productivity of ethanol.

TABLE 12
Ethanol Concentration for Fermentation at Different pHs.

| | BR1 | BR2 | BR3 |
|---|---|---|---|
| time, h | pH 7.5 | pH 7 | pH 6.5 |
| 0 | 0.04 | 0.00 | 0.17 |
| 20.5 | 2.68 | 4.19 | 4.22 |
| 48.5 | 6.15 | 9.80 | 10.7 |
| 68.5 | 9.00 | 13.0 | 13.5 |
| 92.5 | 11.9 | 15.3 | 15.3 |
| 116.5 | 11.6 | 15.4 | 15.3 |
| 144.5 | 11.5 | 13.5 | 16.1 |
| 175.5 | 11.8 | 15.6 | 16.4 |

### Example 8

### Reduced pH in the Presence of Canola Oil

Reactors contained 300 mL media containing 50 g/L cellobiose, 3 g/L K₂HPO₄, 1.6 g/L KH₂PO₄, 2 g/L TriSodium citrate.2H₂O, 1.2 g/L citric acid H₂O, 0.5 g/L (NH₄)₂SO₄, 1 g/L NaCl, 0.8 g/L MgCl₂.6H₂O, 0.1 g/L CaCl₂.2H₂O, 0.00125 g/L FeSO₄.7H₂O, 1 g/L Cysteine HCl, 10 g/L yeast extract (Bacto), along with 5 g/L of corn steep powder dissolved in DI water. The fermentors were operated under batch mode.

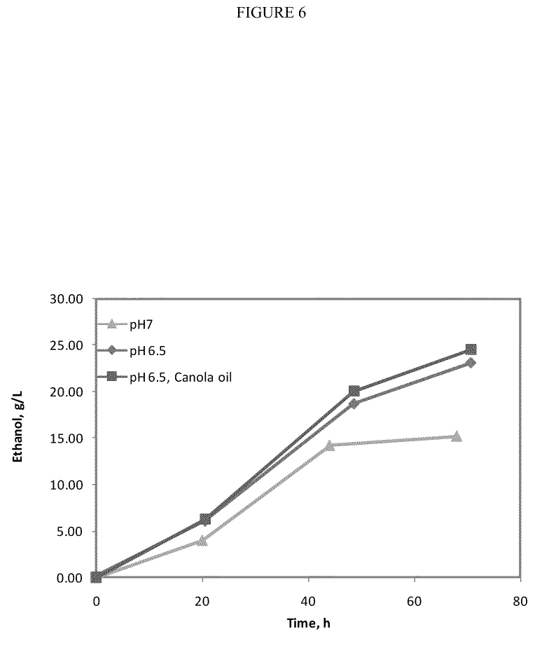

The fermenters were monitored for ethanol concentration. The results are shown in Table 13. A higher ethanol concentration and production resulted from operation at low pH in the presence of canola oil, as well as improved titer and productivity for operation at pH 6.5 as compared to operation at 7.0 (FIG. 6).

TABLE 13
Fermentation at variable pH with Canola Oil Present.

| | | pH = 6.5, | | |
|---|---|---|---|---|
| | | Canola | | |
| Time, h | pH = 6.5 | oil | time, h | pH = 7 |
| 0 | 0.18 | 0.03 | 0 | 0.00 |
| 20.5 | 6.07 | 6.26 | 20 | 4.05 |
| 48.5 | 18.67 | 20.02 | 44 | 14.22 |
| 70.5 | 23.08 | 24.51 | 68 | 15.20 |

TABLE 14
Fatty Acid Profile of Various Edible Fats and Oils; Values as Percent of Total Fatty Acids.

| | | | | | | | | Poly unsaturated | |
|---|---|---|---|---|---|---|---|---|---|
| | | | | | | | Mono | | Alpha |
| | | Saturated | | | | | unsaturated | Linoleic | Linolenic |
| | | Capric | Lauric | Myristic | Palmitic | Stearic | Oleic | Acid | Acid |
| | Unsat./Sat. | Acid | Acid | Acid | Acid | Acid | Acid | (ω6) | (ω3) |
| Oil or Fat | ratio | C10:0 | C12:0 | C14:0 | C16:0 | C18:0 | C18:1 | C18:2 | C18:3 |
| Almond Oil | 9.7 | — | — | — | 7 | 2 | 69 | 17 | — |
| Beef Tallow | 0.9 | — | — | 3 | 24 | 19 | 43 | 3 | 1 |
| Butterfat (cow) | 0.5 | 3 | 3 | 11 | 27 | 12 | 29 | 2 | 1 |
| Butterfat (goat) | 0.5 | 7 | 3 | 9 | 25 | 12 | 27 | 3 | 1 |
| Butterfat | 1 | 2 | 5 | 8 | 25 | 8 | 35 | 9 | 1 |
| (human) | | | | | | | | | |
| Canola Oil | 15.7 | — | — | — | 4 | 2 | 62 | 22 | 10 |
| Cocoa Butter | 0.6 | — | — | — | 25 | 38 | 32 | 3 | — |
| Cod Liver Oil | 2.9 | — | — | 8 | 17 | — | 22 | 5 | — |
| Coconut Oil | 0.1 | 6 | 47 | 18 | 9 | 3 | 6 | 2 | — |
| Corn Oil (Maize | 6.7 | — | — | — | 11 | 2 | 28 | 58 | 1 |
| Oil) | | | | | | | | | |
| Cottonseed Oil | 2.8 | — | — | 1 | 22 | 3 | 19 | 54 | 1 |
| Flaxseed Oil | 9 | — | — | — | 3 | 7 | 21 | 16 | 53 |
| Grape seed Oil | 7.3 | — | — | — | 8 | 4 | 15 | 73 | — |
| Lard (Pork fat) | 1.2 | — | — | 2 | 26 | 14 | 44 | 10 | — |
| Olive Oil | 4.6 | — | — | — | 13 | 3 | 71 | 10 | 1 |
| Palm Oil | 1 | — | — | 1 | 45 | 4 | 40 | 10 | — |
| Palm Olein | 1.3 | — | — | 1 | 37 | 4 | 46 | 11 | — |
| Palm Kernel Oil | 0.2 | 4 | 48 | 16 | 8 | 3 | 15 | 2 | — |
| Peanut Oil | 4 | — | — | — | 11 | 2 | 48 | 32 | — |
| Safflower Oil* | 10.1 | — | — | — | 7 | 2 | 13 | 78 | — |
| Sesame Oil | 6.6 | — | — | — | 9 | 4 | 41 | 45 | — |
| Soybean Oil | 5.7 | — | — | — | 11 | 4 | 24 | 54 | 7 |
| Sunflower Oil* | 7.3 | — | — | — | 7 | 5 | 19 | 68 | 1 |
| Walnut Oil | 5.3 | — | — | — | 11 | 5 | 28 | 51 | 5 |

### Example 10

### Genetic Modification of Clostridium phytofermentans to Increase Production of Ethanol, Other Biofuels and Chemical Products

Plasmids suitable for use in C. phytofermentans were constructed using portions of plasmids obtained from bacterial culture collections. Plasmid Pimp1 is a non-conjugal plasmid that can replicate in E. coli as well as a range of gram-positive bacterial species and it also encodes for resistance to erythromycin. C. phytofermentans is highly sensitive to erythromycin being unable to grow at concentrations of 0.5 micrograms of erythromycin per ml of microbial growth media. The broad host range conjugal plasmid RK2 contains all of the genes needed for a bacterial conjugation system which include: an origin of replication specific to the DNA polymerase of the conjugation system, conjugal DNA replication genes, and genes encoding for the synthesis of pili to enable the recognition of potential recipient bacterial cells and to serve as the conduit through which single-stranded plasmid DNA is transferred by cell-to-cell contact from donor to recipient cells. The origin of transfer for the RK2 conjugal system was acquired from plasmid Prk290 which was obtained from the German Collection of Microorganisms and Cell Cultures (DSMZ) as DSM 3928, and the other conjugation functions of RK2 were acquired from Prk2013 which was obtained from DSMZ as DSM 5599. The polymerase chain reaction was used to amplify the 112 base pair origin of transfer region (oriT) from Prk290 using primers that added Cla1 restriction sites flanking the oriT region. This DNA fragment was inserted into the Cla1 site of pIMP1 to yield plasmid Pimpt. Pimpt was shown to be transferable from one strain of E. coli to another when Prk2013 was also present to supply other conjugation functions. However, Pimpt could not be demonstrated to be conjugally transferred—from E. coli to C. phytofermentans. Because the promoter driving the expression of the erythromycin resistance gene in Pimpt might not function in C. phytofermentans PCR was used to amplify the promoter of the alcohol dehydrogenase gene C. phytofermentans 1029 from the C. phytofermentans chromosome and it was used to replace the promoter of the erythromycin gene in Pimpt to create Pimpt1029. When Prk2013 is also present to supply other conjugation functions, Pimpt1029 could be conjugally transferred from E. coli to C. phytofermentans. Successful transfer of plasmid DNA into C. phytofermentans was demonstrated by virtue of the ability of the C. phytofermentans derivative containing Pimpt1029 to grow on media containing up to 10 micrograms per ml erythromycin and by use of PCR primers to specifically amplify two genetic regions specific to Pimpt1029 from the C. phytofermentans derivative but not from a control C. phytofermentans culture that does not contain the plasmid.

Conjugal transfer of Pimpt1029 from E. coli to C. phytofermentans is accomplished by initially constructing an E. coli strain (DHSalpha) that contains both Pimpt1029 and Prk2013. Then fresh cells of this E. coli culture and fresh cells of the C. phytofermentans recipient culture are obtained by growth to mid-log phase using appropriate growth media (L broth and QM1 media respectively). The two bacterial cultures are then centrifuged to yield cell pellets and the pellets resuspended in the same media to obtain cell suspensions that concentrated about ten-fold and having cell densities of about 10¹⁰ cells per ml. These concentrated cell suspensions are then mixed to achieve a donor-to-recipient ratio of five-to-one. Following this, the cell suspension was spotted onto QM1 agar plates and incubated anaerobically at 30 degrees Centigrade for 24 hours. The cell mixture was removed from the QM1 plate and placed on solid or in liquid QM1 media containing antibiotics chosen to allow the survival of only C. phytofermentans recipient cells that express erythromycin resistance. This was accomplished by using a combination of antibiotics that consisted of trimethoprim at 20 micrograms per ml, cycloserine at 250 micrograms per ml, and erythromycin at 10 micrograms per ml. The E. coli donor was unable to survive exposure to these concentrations of trimethoprim and cycloserine, while the C. phytofermentans recipient was unable to survive exposure to this concentration of erythromycin (but could tolerate the concentrations of trimethoprim and cycloserine). Accordingly, after incubation of these antibiotic-containing plates or liquid media for 5-to-7 days at 30 degrees Centigrade under anaerobic conditions, derivates of C. phytofermentans were obtained that were erythromycin resistant and these derivatives were subsequently shown to contain Pimpt1029 as demonstrated by PCR analyses.

The surprising result was that the only a specially constructed derivative of the erythromycin resistance gene that contained the C. phytofermentans promoter from the alcohol dehydrogenase gene could be functionally expressed in C. phytofermentans.

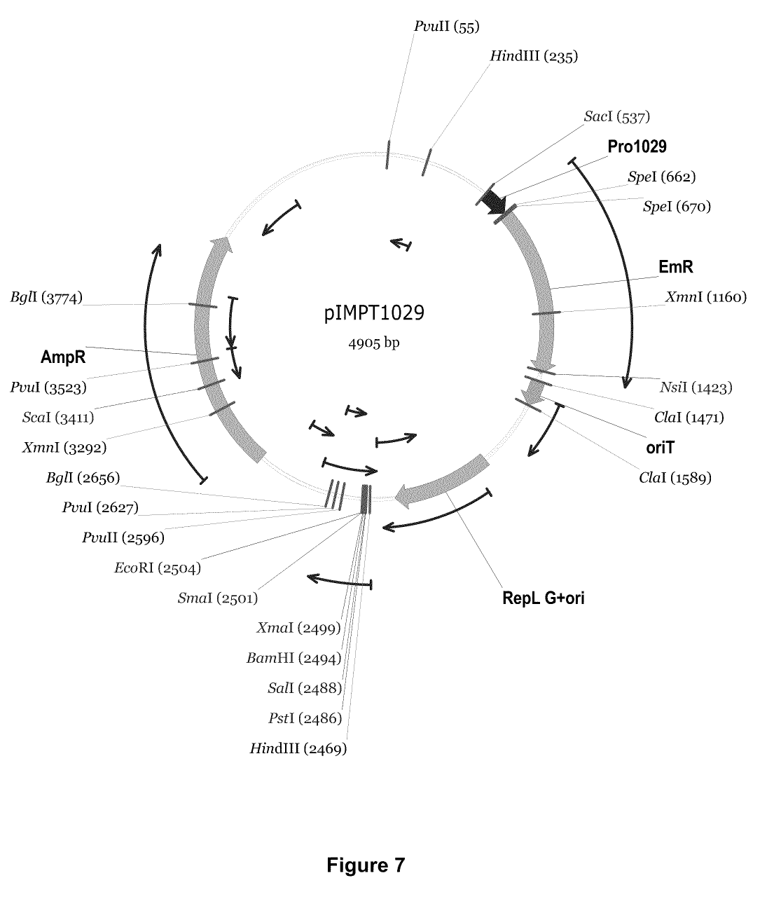

Other genes of interest, either from C. phytofermentans or from heterologous sources are introduced into the Pimpt construct and used to transform C. phytofermentans and, hence, these gene products are useful to increase production of saccharolytic enzymes, hexose transport proteins, and hexose metabolism and enzymes used in the conversion of fermentation intermediates into alcohol final products and other biofuels of C. phytofermentans. A map of the plasmid Pimpt1029 is shown in FIG. 7.

All references cited herein, including but not limited to published and unpublished applications, patents, and literature references, and also including but not limited to the references listed in the Appendix, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that can vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

What is claimed is:

1. A method for producing a fermentive end-product comprising:
culturing a medium comprising Clostridium for a first period of time under conditions suitable for production of a fermentive end-product by said;
adding one or more nutrients to the medium comprising Clostridium while prior to harvesting the fermentive end product;
culturing a medium comprising Clostridium for a second period of time; and
harvesting a fermentive end-product from the medium.

2. The method of claim 1, wherein the Clostridium strain is Clostridium phytofermentans.

3. The method of claim 1, wherein the fermentive end-product is ethanol.

4. The method of claim 1, wherein the medium comprises a cellulosic and/or lignocellulosic material.

5. The method of claim 4, wherein the cellulosic or lignocellulosic material is not enzymatically treated with a sufficient quantity of enzymes to convert more than 15% of the cellulosic or lignocellulosic material to simple sugars within 24 hours.

6. A method of producing a fermentive end product comprising the steps of:
culturing a strain of Clostridium phytofermentans in a medium;
maintaining the total concentration of sugar compounds in the medium at least about 18 g/L; and
harvesting a fermentive end-product from the medium.

7. The method of claim 6, wherein maintaining the total concentration of sugar compounds comprises adding one or more medium components, at least one of which comprises one or more sugar compounds to the medium at least once during the culturing, wherein the medium components are added to a vessel containing the culture.

8. The method claim 6, wherein the total concentration of sugar compounds in the medium is maintained within the range from about 1 g/L to about 100 g/L for a portion of the culturing.

9. The method of claim 6, wherein the total concentration of sugar compounds in the medium varies by less than about 25% during the period of fermentive end product production.

10. The method of claim 6, wherein the fermentive end-product is ethanol.

11. The method of claim 6, further comprising adding a medium component comprising one or more nitrogen-containing material to the medium at least once during the fermentation, and wherein the medium component is added to a vessel containing the culture.

12. The method of claim 11, wherein one or more of the medium components comprises one or more nitrogen-containing material.

13. The method of claim 6, wherein the medium comprises a cellulosic or lignocellulosic material.

14. The method of claim 13, wherein the cellulosic or lignocellulosic material is not enzymatically treated with a sufficient quantity of enzymes to convert more than 15% of the cellulosic or lignocellulosic material to simple sugars within 24 hours.

15. A method of producing a fermentive end product, the method comprising the steps of:
culturing a strain of Clostridium in a medium; and
adding one or more medium components to the medium during the culturing of the Clostridium wherein one or more of the medium components comprises one or more sugar compounds, and the one or more sugar compounds are added in relation to an amount of sugar converted by the Clostridium to other compounds.

16. The method of claim 15, wherein one or more of the medium components comprises a nitrogen source.

17. The method of claim 16, wherein the nitrogen source includes proline, glycine, histidine, and/or isoleucine.

18. The method of claim 15, wherein one or more of the medium components comprises a cellulosic or lignocellulosic material.

19. The method of claim 18, wherein the cellulosic or lignocellulosic material is not enzymatically treated with a sufficient quantity of enzymes to convert more than 15% of the cellulosic or lignocellulosic material to simple sugars within 24 hours.

20. A method of producing a fermentive end product, the method comprising:
adding a first inoculum of a strain of Clostridium to a medium;
culturing the Clostridium under conditions suitable for production of ethanol;
adding additional viable cells of Clostridium sp. to the medium more than five hours after the first inoculum of Clostridium is added to the medium; and
harvesting the fermentive end product from the medium.

21. The method of claim 19, further comprising adding one or more media components to the medium after adding the first inoculum of Clostridium.

22. The method of claim 19, wherein an addition of media components and an addition of viable cells occurs sequentially or simultaneously.

23. A method of producing ethanol, the method comprising the steps of:
removing an impurity from an impure ethanol material to produce a purified ethanol material, wherein the purified ethanol material is more than about 90% (wt.) ethanol, and the impure ethanol material is derived from a fermentation medium made by culturing Clostridium phytofermentans cells in a fed batch culture, and wherein the ethanol concentration in the fermentation medium is greater than about 7 g/L.

24. A method of producing a fermentive end product, the method comprising the steps of: culturing a medium comprising a strain of Clostridium phytofermentans, wherein the fermentive end product is produced at an instantaneous productivity of at least about 3 g/L-day.

25. A method of producing a fermentive end product, comprising:
providing a cellulosic material, wherein said cellulosic material has not been treated with exogenously supplied chemicals or enzymes; combining the cellulosic material with a microbe in a medium, wherein the medium does not comprise exogenously supplied enzymes; and fermenting the cellulosic material under conditions and for a time sufficient to produce a fermentive end product.

26. A method of producing a fermentive end product, the method comprising: fermenting cells of Clostridium phytofermentans in the presence of a pH modifier, wherein a fermentive end product is produced.

27. The method of claim 26, wherein the fermentive end product is ethanol.

28. The method of claim 26, wherein fermenting the cells occurs at a pH, between about 6.0 to about 7.2.

29. The method of claim 28, wherein the pH is about 6.5.

30. A method of producing a fermentive end product, the method comprising: fermenting cells of a Clostridium strain in the presence of an added fatty acid material, wherein a fermentive end product is produced.

31. The method of claim 30, wherein the fatty acid comprising material comprises one or more of corn oil, sunflower oil, safflower oil, canola oil, soybean oil, or rape seed oil.

32. The method of claim 30, wherein the fatty acid comprising material comprises a phospholipid or a lysophospholipid.

33. A fermentation medium, the medium comprising cells of Clostridium phytofermentans and a pH modifier, wherein a fermentive end product is produced.

34. A fermentation medium, the medium comprising cells of a Clostridium strain and an added fatty acid containing compound, wherein a fermentive end product is produced.

35. A fermentation medium comprising a strain of Clostridium phytofermentans, a nitrogen source comprising proline, glycine, histidine, and/or isoleucine, and a cellulosic or lignocellulosic material.

36. A method of producing alcohol, the method comprising: fermenting cells of a Clostridium strain and the presence of a pH modifier and a fatty acid material, wherein a fermentive end product is produced.

37. A fuel plant comprising a fermenter configured to house a medium and a strain of Clostridium phytofermentans, wherein said fermenter is configured to maintain an amount of sugar compounds at a level that varies by less than about 25% during fermentation.

38. A fuel plant comprising a fermenter configured to house a medium and a strain of Clostridium phytofermentans, wherein said fermenter is configured to periodically supplement said medium with additional medium components or additional viable cells of Clostridium phytofermentans.

39. A fuel plant comprising a fermenter configured to house a medium and a strain of Clostridium phytofermentans, wherein said medium comprises a pH modifier and a cellulosic or lignocellulosic material.

40. The fuel plant of claim 39, wherein said medium further comprises a fatty acid material.

41. A fuel plant comprising a fermenter configured to house a medium and a strain of Clostridium phytofermentans, wherein said medium comprises a nitrogen source comprising proline, glycine, histidine, and/or isoleucine, and a cellulosic or lignocellulosic material.

42. A fuel plant comprising a fermenter configured to house a medium and a strain of Clostridium phytofermentans, wherein said medium comprises a fatty acid material and a cellulosic or lignocellulosic material.

43. A fermentive end product produced by fermenting a cellulosic or lignocellulosic material with a strain of Clostridium phytofermentans, in a medium comprising an amount of sugar compounds at a level that varies by less than about 25% during fermentation.

44. A fermentive end product produced by fermenting a cellulosic or lignocellulosic material with a strain of Clostridium phytofermentans, in a medium comprising a pH modifier.

45. A fermentive end product produced by fermenting a cellulosic or lignocellulosic material with a strain of Clostridium phytofermentans, in a medium comprising a fatty acid.

46. A fermentive end product produced by fermenting a cellulosic or lignocellulosic material with a strain of Clostridium phytofermentans, in a medium comprising a nitrogen source comprising proline, glycine, histidine, and/or isoleucine.

47. The fermentive end product of claims 43-46, wherein said fermentive end product is ethanol.
